(19) United States
(12) Patent Application Publication
(10) Pub. No.: US 2026/0249072 A1
(43) Pub. Date: Aug. 27, 2026

(54) CIRCULATORY ASSIST DEVICE WITH MULTI-LEAFLET VALVE

(71) Applicant: CardiacBooster B.V., Nijmegen (NL)

(72) Inventors: David O'Reilly, Galway (IE); Shane Mulderrig, Galway (IE); Anisia Lauditi, Nijmegen (NL); Ferry van der Linde, Schijndel (NL); Patrick Griffin, Galway (IE); Daniël Immanuel Michaël van Dort, Nijmegen (NL); Amit Tubishevitz, Tel-Aviv (IL); Fran McCormack, Galway (IE); Hubert Creaven, Galway (IE)

(21) Appl. No.: 19/650,604

(22) Filed: Apr. 17, 2026

Related U.S. Application Data
(63) Continuation of application No. PCT/IB2024/060293, filed on Oct. 19, 2024.
(60) Provisional application No. 63/591,900, filed on Oct. 20, 2023.

(51) Int. Cl. A61M 60/896; A61M 60/13; A61M 60/139; A61M 60/268; A61M 60/295; A61M 60/497; A61M 60/843; A61M 60/865

(52) U.S. Cl. CPC A61M 60/896; A61M 60/13; A61M 60/139; A61M 60/268; A61M 60/295; A61M 60/497; A61M 60/843; A61M 60/865

(57) ABSTRACT

In some variations, a circulatory assist system may include a pump body comprising a conduit having an inflow region and an outflow region, and a volume displacement member arranged in the conduit and operable between an expansion phase and a contraction phase. The circulatory assist system may also include an inlet valve arranged in the inflow region and comprising a plurality of leaflets. The inlet valve may receive blood into the conduit and the pump body may convey the received blood through the outflow region via cyclical operation of the volume displacement member between the expansion phase and the contraction phase. In some variations, the inlet valve may operate between open and closed states at high frequencies while withstanding a high pressure differential when the circulatory assist system is in use.

### CROSS-REFERENCE TO RELATED APPLICATIONS

The present application is a continuation of International Application No. PCT/IB2024/060293, filed Oct. 19, 2024, which claims the benefit of priority to U.S. Provisional Patent Application No. 63/591,900, filed Oct. 20, 2023, each of which is incorporated by reference herein in its entirety.

### TECHNICAL FIELD

The present technology relates to a circulatory assist device with a multi-leaflet valve.

### BACKGROUND

For patients suffering from cardiogenic shock, or those undergoing high-risk percutaneous coronary interventions (PCI), a patient's heart function may be compromised such that the use of circulatory assist devices may be required to maintain adequate blood flows through the circulatory system. Although there is some variation depending on patient size and condition, circulatory assist devices for patients undergoing high risk PCI typically must produce blood flows of least 3 L/min to maintain adequate circulation, while for patients in cardiogenic shock, a minimum of 5 L/min is generally considered necessary.

The most common types of circulatory assist devices are intra-aortic balloon pumps (IABP), extra-corporeal membrane oxygenation (ECMO) systems, and impeller-based blood pumps. IABPs are catheters having an inflatable balloon which can be placed in the descending aorta and cyclically inflated to displace the blood. ECMO systems include a venous catheter for removing deoxygenated blood from the venous system, an extracorporeal oxygenator and pump, and an arterial catheter for returning the blood to the arterial system, thus bypassing the heart. Impeller pump systems have a rotary impeller that can be placed in a chamber of the heart or in a major vessel and rotated at relatively high speed to propel blood through the circulatory system.

While offering some benefit in increasing blood flow and reducing load on the heart, currently available circulatory assist devices suffer from certain drawbacks. IABPs may not improve flows adequately to support the patient when the heart is significantly compromised, such as during cardiogenic shock. ECMO systems may have higher morbidity associated with multiple catheterizations including bleeding, thrombus, and infection, as well as problems associated with membrane oxygenation including cognitive deficit and stroke. In addition, they increase afterload which is generally regarded as counterproductive. Impeller pump systems, if operated at higher speeds in order to produce higher flows, can result in excessive hemolysis; further, if impeller pumps are made larger to produce higher flows, the profile of such devices can be undesirably large, inhibiting percutaneous delivery, and increasing the risk of injury to cardiovascular structures and/or causing limb ischemia. As a result, current impeller-type pumps which are capable of providing the high flows necessary for patients in cardiogenic shock, are often too large for endovascular delivery thus requiring surgical placement, and further produce undesirable levels of hemolysis. What is needed, therefore, are improved circulatory support systems and methods.

### SUMMARY

The subject technology is illustrated, for example, according to various aspects described below, including with reference to FIGS. 1A-31B. Various examples of aspects of the subject technology are described as numbered clauses (1, 2, 3, etc.) for convenience. These are provided as examples and do not limit the subject technology.

1. A blood pump device comprising:
a pump body comprising:
   a conduit having an inflow region and an outflow region; and
   an inlet valve arranged in the inflow region and comprising a plurality of leaflets,
wherein at least one leaflet comprises a base region comprising a first bending stiffness and an edge region comprising a second bending stiffness, wherein the second bending stiffness is lower than the first bending stiffness.

2. The blood pump device of clause 1, wherein the at least one leaflet comprises an intermediate region between the base region and the edge region comprising a third bending stiffness, wherein the third bending stiffness is between the first bending stiffness and the second bending stiffness.

3. The blood pump device of clause 1 or 2, wherein the at least one leaflet exhibits a linear transition in bending stiffness between the first bending stiffness and the second bending stiffness.

4. The blood pump device of any one of clauses 1-3, wherein the at least one leaflet exhibits a stepwise transition in bending stiffness between the first bending stiffness and the second bending stiffness.

5. The blood pump device of any one of clauses 1-4, wherein the base region comprises a first leaflet thickness and the edge region comprises a second leaflet thickness, wherein the second leaflet thickness is less than the first leaflet thickness.

6. The blood pump device of clause 5, wherein the second leaflet thickness is between about 45% and about 80% of the first leaflet thickness.

7. The blood pump device of any one of clauses 1-6, wherein the base region comprises a first material, and the edge region comprises a second material, wherein the second material has a lower durometer than the first material.

8. The blood pump device of clause 7, wherein the first material has a durometer of greater than 55 D, and the second material has a durometer of lower than 55 D.

9. The blood pump device of any one of clauses 1-8, wherein at least one leaflet comprises multiple layers of material.

10. The blood pump device of any one of clauses 1-9, wherein the plurality of leaflets are formed from a single continuous membrane.

11. The blood pump device of any one of clauses 1-10, wherein the plurality of leaflets are formed from multiple discrete membranes.

12. The blood pump device of any one of clauses 1-11, wherein the plurality of leaflets comprises three leaflets.

13. The blood pump device of any one of clauses 1-12, wherein the inlet valve has an outer diameter of about 25 mm or less.

14. The blood pump device of any one of clauses 1-13, wherein the inlet valve has an outer diameter of about between about 10 mm and about 15 mm.

15. The blood pump device of any one of clauses 1-14, wherein the inlet valve in its open state has a valve orifice area between about 15% and about 90% of a maximum cross-sectional area of the conduit, as measured at a location of the inlet valve.

16. The blood pump device of any one of clauses 1-15, wherein the inlet valve has a cutoff angle of between about 0 degrees and about 45 degrees.

17. The blood pump device of any one of clauses 1-16, further comprising a guidewire extending through the inlet valve.

18. The blood pump device of any one of clauses 1-17, wherein the inlet valve is operable in an open state and a closed state.

19. The blood pump device of clause 18, wherein when the inlet valve is in the closed state, the leaflets define an aperture therebetween, wherein the guidewire extends through the aperture.

19. The blood pump device of clause 18, wherein a shape of the aperture is complementary to a cross-sectional profile of the guidewire or a cross-sectional profile of a guidewire housing member that receives the guidewire.

20. The blood pump device of any one of clauses 1-19, further comprising a rigid support member that receives the guidewire, a guidewire housing member that receives the guidewire, or both.

21. The blood pump device of clause 20, wherein the rigid support member extends through the inlet valve.

22. The blood pump device of any one of clauses 1-21, wherein the conduit comprises at least one membrane.

23. The blood pump device of clause 22, wherein the conduit further comprises an expandable support and the at least one membrane is covering at least a portion of an inner surface or outer surface of the expandable support.

24. The blood pump device of clause 23, wherein at least one of the plurality of leaflets is joined to the at least one membrane.

25. The blood pump device of clause 23 or 24, wherein the at least one membrane comprises an inner membrane covering the inner surface of the expandable support and an outer membrane covering the outer surface of the expandable support, and at least one of the plurality of leaflets is joined to the inner membrane or the outer membrane, or both.

26. The blood pump device of clause 25, wherein a proximal end of the expandable support is coupled to a catheter extending through the pump body.

27. The blood pump device of clause 25 or 26, wherein a distal end of the expandable support is longitudinally movable relative to the catheter.

28. The blood pump device of any one of clauses 1-27, wherein the pump body further comprises a volume displacement member arranged in the conduit and having an expandable volume cyclically operable between an expansion phase and a contraction phase.

29. The blood pump device of clause 28, wherein a distalmost end of the expandable volume of the volume displacement member is separated from the inlet valve by a distance of between about 1 mm and about 35 mm.

30. The blood pump device of clause 28, wherein at least a portion of the volume displacement member extends through the inlet valve.

31. The blood pump device of any one of clauses 28-30, wherein the volume displacement member comprises a balloon.

32. The blood pump device of any one of clauses 1-31, wherein the inlet valve has an upstream side and a downstream side, and is operable in a valve cycle comprising:
an open state in which the leaflets permit fluid flow between the upstream side and the downstream side; and
a closed state in which the leaflets substantially prevent fluid flow from the downstream side to the upstream side.

33. The blood pump device of clause 32, wherein the inlet valve is configured to maintain the closed state under a pressure differential of up to at least about 500 mmHg from the downstream side to the upstream side, without allowing fluid leakage from the downstream side to the upstream side.

34. The blood pump device of clause 33, wherein the valve is configured to maintain the closed state under a pressure differential of up to at least about 700 mmHg from the downstream side to the upstream side, without allowing blood leakage from the downstream side to the upstream side.

35. The blood pump device of any one of clauses 32-34, wherein the valve is configured to repeatedly cycle between the open state and the closed state at a frequency of between about 500 beats per minute and about 5000 beats per minute.

36. The blood pump device of clause 35, wherein the valve is configured to be cyclically operated at a frequency of between about 1000 beats per minute and about 5000 beats per minute.

37. The blood pump device of any one of clauses 32-36, wherein the valve is configured to transition from the closed state to the open state over no more than about 20% of the duration of the valve cycle.

38. A method, comprising:
positioning a blood pump device in the circulatory system of a patient, wherein the blood pump device comprises:
   a conduit having an inflow region and an outflow region:
   a volume displacement member arranged in the conduit; and
   an inlet valve arranged in the inflow region and comprising a plurality of leaflets, an upstream side, and a downstream side; and
cyclically operating the volume displacement member between an expansion phase and a contraction phase;
wherein the inlet valve transitions between an open state during the contraction phase to allow blood flow into the conduit, and a closed state during the expansion phase in which the valve substantially prevents blood flow out of the conduit through the inflow region, and
wherein during the expansion phase, the inlet valve maintains the closed state under a pressure differential of up to at least about 500 mmHg from the downstream side to the upstream side, without allowing blood leakage from the downstream side to the upstream side.

39. The method of clause 38, wherein the valve maintains the closed state under a pressure differential of up to at least about 700 mmHg from the downstream side to the upstream side, without allowing blood leakage from the downstream side to the upstream side.

40. The method of clause 38 or 39, wherein positioning the blood pump device comprises positioning at least a portion of the inflow region of the conduit in a left ventricle of the patient.

41. The method of clause 40, further comprising positioning at least a portion of the outflow region of the conduit in an aorta of the patient when at least a portion of the inflow region of the conduit is in the left ventricle.

42. The method of any one of clauses 38-41, wherein positioning the blood pump device comprises positioning the inlet valve in a plane offset from a native valve plane.

43. The method of clause 42, wherein the native valve plane is an aortic valve plane.

44. The method of clause 38 or 39, wherein positioning the blood pump device comprises positioning the entire blood pump device in a blood vessel of the patient.

45. The method of any one of clauses 38-49, wherein the inlet valve in its open state has a valve orifice area between about 15% and about 90% of a maximum cross-sectional area of the conduit, as measured at a location of the inlet valve.

46. The method of any one of clauses 38-45, wherein the inlet valve comprises a cylindrical valve body and the plurality of leaflets are coupled to the valve body.

47. The method of clause 46, wherein the conduit has a first inner diameter and the valve body has a second inner diameter, the second inner diameter being between about 70% and about 98% of the first inner diameter.

48. The method of any one of clauses 38-47, wherein cyclically operating the volume displacement member comprises cyclically operating the volume displacement member at a frequency of at least about 500 beats per minute.

49. The method of clause 48, wherein cyclically operating the volume displacement member comprises cyclically operating the volume displacement member at a frequency of between about 1000 beats per minute and about 5000 beats per minute.

50. The method of any one of clauses 38-49, wherein positioning the blood pump device comprises collapsing the blood pump device in a lumen of a delivery catheter, and inserting the delivery catheter in a blood vessel.

51. The method of clause 50, wherein the lumen of the delivery catheter is no more than about 6 mm in diameter.

52. The method of claim 50 or 51, wherein positioning the blood pump device further comprises deploying the blood pump device from the lumen of the delivery catheter at a pumping location in a heart chamber or blood vessel.

53. The method of clause 52, wherein the inlet valve self-expands from a collapsed valve configuration in the lumen of the delivery catheter to an expanded valve configuration as the blood pump device is deployed.

54. The method of clause 53, wherein the inlet valve in the expanded valve configuration has an expanded diameter that is between about 1.2 and about 6 times the diameter of the lumen of the delivery catheter.

55. The method of any one of clauses 38-54, wherein the valve is operable in a valve cycle comprising the open state and the closed state, and is configured to transition from the closed state to the open state over no more than about 20% of the duration of the valve cycle.

56. A valve arrangement for a blood pump device, the valve arrangement comprising:
a valve comprising a plurality of leaflets, an upstream side, and a downstream side, wherein the valve is operable in:
   an open state in which the leaflets permit fluid flow between the upstream side and the downstream side; and
   a closed state in which the leaflets substantially prevent fluid flow from the downstream side to the upstream side,
wherein the valve is configured to maintain the closed state under a pressure differential of up to at least about 500 mmHg from the downstream side to the upstream side, without allowing fluid leakage from the downstream side to the upstream side.

57. The valve arrangement of clause 56, wherein the valve is configured to maintain the closed state under a pressure differential of up to at least about 700 mmHg from the downstream side to the upstream side, without allowing fluid leakage from the downstream side to the upstream side.

58. The valve arrangement of clause 56 or 57, wherein the valve is configured to be cyclically operated between the open state and the closed state at a frequency of at least about 500 beats per minute.

59. The valve arrangement of clause 58, wherein the valve is configured to be cyclically operated at a frequency of between about 1000 beats per minute and about 5000 beats per minute.

60. The valve arrangement of any one of clauses 56-59, wherein the valve is cyclically operable in a valve cycle comprising the open state and the closed state, wherein the valve is configured to transition from the closed state to the open state over no more than about 20% of the duration of the valve cycle.

61. The valve arrangement of any one of clauses 56-60, wherein at least one leaflet comprises a base region comprising a first bending stiffness and an edge region comprising a second bending stiffness, wherein the second bending stiffness is lower than the first bending stiffness.

62. The valve arrangement of any one of clauses 56-61, wherein the base region comprises a first leaflet thickness and the edge region comprises a second leaflet thickness, wherein the first leaflet thickness is less than the second leaflet thickness.

63. The valve arrangement of any one of clauses 56-62, wherein the base region comprises a first material, and the edge region comprises a second material, wherein the second material has a greater durometer than the first material.

64. The valve arrangement of any one of clauses 56-63, wherein at least one leaflet comprises multiple layers of material.

65. The valve arrangement of any one of clauses 56-64, wherein the inlet valve has an outer diameter of about 25 mm or less.

66. A blood pump device comprising:
a conduit having an inflow region and an outflow region;
a volume displacement member arranged in the conduit; and
the valve arrangement of any one of clauses 56-65, wherein the valve arrangement is positioned in the inflow region of the conduit.

67. The blood pump device of clause 66, wherein the conduit comprises an expandable support and at least one membrane covering at least a portion of the expandable support.

68. The blood pump device of clause 67, wherein the at least one membrane covers an inner surface or outer surface of the expandable support.

69. The blood pump device of clause 67 or 68, wherein at least one of the plurality of leaflets is joined to the at least one membrane.

70. The blood pump device of clause 68 or 69, wherein the at least one membrane comprises an inner membrane covering the inner surface of the expandable support and an outer membrane covering the outer surface of the expandable support, and at least one of the plurality of leaflets is joined to the inner membrane or the outer membrane, or both.

71. The blood pump device of any one of clauses 66-70, further comprising a tubular member extending longitudinally through the conduit and between the leaflets of the valve.

72. The blood pump device of clause 71, wherein the tubular member is configured to slidably receive a guidewire.

73. The blood pump device of clause 71 or 72, wherein the leaflets are configured to coapt with an outer surface of the tubular member.

74. The blood pump device of clause 73, wherein at least one leaflet has an inner edge portion complementary with a cross-sectional profile of the tubular member.

75. The blood pump device of any one of clauses 71-74, wherein the tubular member is configured to support the leaflets to substantially inhibit prolapsing of the leaflets.

76. The blood pump device of any one of clauses 71-75, wherein the tubular member extends through an interior of the volume displacement member.

77. The blood pump device of any one of clauses 66-76, wherein the valve in its open state has a valve orifice area between about 15% and about 90% of a maximum cross-sectional area of the conduit, as measured at a location of the inlet valve.

78. The blood pump device of any one of clauses 66-77, wherein the valve comprises a cylindrical valve body and the plurality of leaflets are coupled to the valve body.

79. The blood pump device of clause 78, wherein the conduit has a first inner diameter and the valve body has a second inner diameter, the second inner diameter being between about 70% and about 98% of the first inner diameter.

80. The blood pump device of any one of clauses 66-79, wherein the blood pump device is collapsible into a lumen of a delivery catheter.

81. The blood pump device of clause 80, wherein the lumen of the delivery catheter is no more than about 6 mm in diameter.

82. The blood pump device of clause 81, wherein the valve is configured to self-expand from a collapsed valve configuration to an expanded valve configuration.

83. The blood pump device of clause 82, wherein the valve in the expanded valve configuration has an expanded diameter that is between about 1.2 and about 6 times the diameter of the lumen of the delivery catheter.

84. A method, comprising:
positioning a blood pump device in the circulatory system of a patient, wherein the blood pump device comprises:
   a conduit having an inflow region and an outflow region;
   a volume displacement member arranged in the conduit; and
   an inlet valve arranged in the inflow region and comprising a plurality of leaflets; and
cyclically operating the volume displacement member between an expansion phase and a contraction phase,
wherein the inlet valve transitions between an open state during the contraction phase to allow blood flow into the conduit, and a closed state during the expansion phase in which the valve substantially prevents blood flow out of the conduit through the inflow region, and
wherein the valve repeatedly cycles between the open state and the closed state at a frequency of between about 500 beats per minute and about 5000 beats per minute.

85. The method of clause 84, wherein the valve is cyclically operated at a frequency of between about 1000 beats per minute and about 5000 beats per minute.

86. The method of clause 84 or 85, wherein the valve remains in the open state during at least a portion of the expansion phase of the volume displacement member.

87. The method of any one of clauses 84-86, wherein during the expansion phase, the inlet valve maintains the closed state under a pressure differential of up to at least about 500 mmHg from the downstream side to the upstream side, without allowing blood leakage from the downstream side to the upstream side.

88. The method of clause 87, wherein the valve maintains the closed state under a pressure differential of up to at least about 700 mmHg from the downstream side to the upstream side, without allowing blood leakage from the downstream side to the upstream side.

89. The method of any one of clauses 84-88, wherein positioning the blood pump device comprises positioning at least a portion of the inflow region of the conduit in a left ventricle of the patient.

90. The method of clause 89, further comprising positioning at least a portion of the outflow region of the conduit in an aorta of the patient when at least a portion of the inflow region of the conduit is in the left ventricle.

91. The method of any one of clauses 84-90, wherein positioning the blood pump device comprises positioning the inlet valve in a plane offset from a native valve plane.

92. The method of clause 91, wherein the native valve plane is an aortic valve plane.

93. The method of any one of clauses 84-88, wherein positioning the blood pump device comprises positioning the entire blood pump device in a blood vessel of the patient.

94. The method of any one of clauses 84-93, wherein the inlet valve in its open state has a valve orifice area between about 15% and about 90% of a maximum cross-sectional area of the conduit, as measured at a location of the inlet valve.

95. The method of any one of clauses 84-94, wherein the inlet valve comprises a cylindrical valve body and the plurality of leaflets are coupled to the valve body.

96. The method of clause 95, wherein the conduit has a first inner diameter and the valve body has a second inner diameter, the second inner diameter being at least between about 70% and about 98% of the first inner diameter.

97. The method of any one of clauses 84-96, wherein cyclically operating the volume displacement member comprises cyclically operating the volume displacement member at a frequency of at least about 500 beats per minute.

98. The method of clause 97, wherein cyclically operating the volume displacement member comprises cyclically operating the volume displacement member at a frequency of between about 1000 beats per minute and about 5000 beats per minute.

99. The method of any one of clauses 84-98, wherein positioning the blood pump device comprises collapsing the blood pump device in a lumen of a delivery catheter, and inserting the delivery catheter in a blood vessel.

100. The method of clause 99, wherein the lumen of the delivery catheter is no more than about 6 mm in diameter.

101. The method of claim 99 or 100, wherein positioning the blood pump device further comprises deploying the blood pump device from the lumen of the delivery catheter at a pumping location in a heart chamber or blood vessel.

102. The method of clause 101, wherein the inlet valve self-expands from a collapsed valve configuration in the lumen of the delivery catheter to an expanded valve configuration as the blood pump device is deployed.

103. The method of clause 102, wherein the inlet valve in the expanded valve configuration has an expanded diameter that is between about 1.2 and about 6 times the diameter of the lumen of the delivery catheter.

104. The method of any one of clauses 84-103, wherein the valve operates over a valve cycle comprising the open state and the closed state, and the valve transitions from the closed state to the open state over no more than about 20% of the duration of the valve cycle.

105. A valve arrangement for a blood pump device, the valve arrangement comprising:
a valve comprising a plurality of leaflets, an upstream side, and a downstream side, wherein the valve is operable in:
   an open state in which the leaflets permit fluid flow between the upstream side and the downstream side; and
   a closed state in which the leaflets substantially prevent fluid flow from the downstream side to the upstream side,
wherein the valve is configured to repeatedly cycle between the open state and the closed state at a frequency of between about 500 beats per minute and about 5000 beats per minute.

106. The valve arrangement of clause 105, wherein the valve is configured to be cyclically operated at a frequency of between about 1000 beats per minute and about 5000 beats per minute.

107. The valve arrangement of clause 105 or 106, wherein the valve is configured to maintain the closed state under a pressure differential of up to at least about 500 mmHg from the downstream side to the upstream side, without allowing fluid leakage from the downstream side to the upstream side.

108. The valve arrangement of clause 107, wherein the valve is configured to maintain the closed state under a pressure differential of up to at least about 700 mmHg from the downstream side to the upstream side, without allowing fluid leakage from the downstream side to the upstream side.

109. The valve arrangement of any one of clauses 105-108, wherein the valve is cyclically operable in a valve cycle comprising the open state and the closed state, wherein the valve is configured to transition from the closed state to the open state over no more than about 20% of the duration of the valve cycle.

110. The valve arrangement of any one of clauses 105-109, wherein at least one leaflet comprises a base region comprising a first bending stiffness and an edge region comprising a second bending stiffness, wherein the second bending stiffness is lower than the first bending stiffness.

111. The valve arrangement of any one of clauses 105-110, wherein the base region comprises a first leaflet thickness and the edge region comprises a second leaflet thickness, wherein the first leaflet thickness is less than the second leaflet thickness.

112. The valve arrangement of any one of clauses 105-111, wherein the base region comprises a first material, and the edge region comprises a second material, wherein the second material has a greater durometer than the first material.

113. The valve arrangement of any one of clauses 105-112, wherein at least one leaflet comprises multiple layers of material.

114. The valve arrangement of any one of clauses 105-113, wherein the inlet valve has an outer diameter of about 25 mm or less.

115. A blood pump device comprising:
a conduit having an inflow region and an outflow region;
a volume displacement member arranged in the conduit; and
the valve arrangement of any one of clauses 105-114, wherein the valve arrangement is positioned in the inflow region of the conduit.

116. The blood pump device of clause 115, wherein the conduit comprises an expandable support and at least one membrane covering at least a portion of the expandable support.

117. The blood pump device of clause 116, wherein the at least one membrane covers an inner surface or outer surface of the expandable support.

118. The blood pump device of clause 116 or 117, wherein at least one of the plurality of leaflets is joined to the at least one membrane.

119. The blood pump device of clause 117 or 118, wherein the at least one membrane comprises an inner membrane covering the inner surface of the expandable support and an outer membrane covering the outer surface of the expandable support, and at least one of the plurality of leaflets is joined to the inner membrane or the outer membrane, or both.

120. The blood pump device of any one of clauses 115-119, further comprising a tubular member extending longitudinally through the conduit and between the leaflets of the valve.

121. The blood pump device of clause 120, wherein the tubular member is configured to slidably receive a guidewire.

122. The blood pump device of clause 120 or 121, wherein the leaflets are configured to coapt with an outer surface of the tubular member.

123. The blood pump device of clause 122, wherein at least one leaflet has an inner edge portion complementary with a cross-sectional profile of the tubular member.

124. The blood pump device of any one of clauses 120-123, wherein the tubular member is configured to support the leaflets to substantially inhibit prolapsing of the leaflets.

125. The blood pump device of any one of clauses 120-124, wherein the tubular member extends through an interior of the volume displacement member.

126. The blood pump device of any one of clauses 115-125, wherein the valve in its open state has a valve orifice area between about 15% and about 90% of a maximum cross-sectional area of the conduit, as measured at a location of the inlet valve.

127. The blood pump device of any one of clauses 115-126, wherein the valve comprises a cylindrical valve body and the plurality of leaflets are coupled to the valve body.

128. The blood pump device of clause 127, wherein the conduit has a first inner diameter and the valve body has a second inner diameter, the second inner diameter being at least between about 70% and about 98% of the first inner diameter.

129. The blood pump device of any one of clauses 115-128, wherein the blood pump device is collapsible into a lumen of a delivery catheter.

130. The blood pump device of clause 129, wherein the lumen of the delivery catheter is no more than about 6 mm in diameter.

131. The blood pump device of clause 130, wherein the valve is configured to self-expand from a collapsed valve configuration to an expanded valve configuration.

132. The blood pump device of clause 131, wherein the valve in the expanded valve configuration has an expanded diameter that is between about 1.2 and about 6 times the diameter of the lumen of the delivery catheter.

133. A method, comprising:
positioning a blood pump device in the circulatory system of a patient, wherein the blood pump device comprises:
   a conduit having an inflow region and an outflow region;
   a volume displacement member arranged in the conduit; and
   an inlet valve arranged in the inflow region and comprising a plurality of leaflets; and
cyclically operating the volume displacement member between an expansion phase and a contraction phase,
wherein the inlet valve transitions between an open state during the contraction phase to allow blood flow into the conduit, and a closed state during the expansion phase in which the valve substantially prevents blood flow out of the conduit through the inflow region, and
wherein the inlet valve transitions from the closed state to the open state over no more than about 20% of the duration of the valve cycle.

134. The method of clause 133, wherein the inlet valve remains in the open state over at least about 50% of the duration of the valve cycle.

135. The method of clause 133 or 134, wherein during the expansion phase, the inlet valve maintains the closed state under a pressure differential of up to at least about 500 mmHg from the downstream side to the upstream side, without allowing blood leakage from the downstream side to the upstream side.

136. The method of clause 135, wherein the valve maintains the closed state under a pressure differential of up to at least about 700 mmHg from the downstream side to the upstream side, without allowing blood leakage from the downstream side to the upstream side.

137. The method of any one of clauses 133-136, wherein positioning the blood pump device comprises positioning at least a portion of the inflow region of the conduit in a left ventricle of the patient.

138. The method of clause 137, further comprising positioning at least a portion of the outflow region of the conduit in an aorta of the patient when at least a portion of the inflow region of the conduit is in the left ventricle.

139. The method of any one of clauses 133-138, wherein positioning the blood pump device comprises positioning the inlet valve in a plane offset from a native valve plane.

140. The method of clause 139, wherein the native valve plane is an aortic valve plane.

141. The method of any one of clauses 133-136, wherein positioning the blood pump device comprises positioning the entire blood pump device in a blood vessel of the patient.

142. The method of any one of clauses 133-141, wherein the inlet valve in its open state has a valve orifice area between about 15% and about 90% of a maximum cross-sectional area of the conduit, as measured at a location of the inlet valve.

143. The method of any one of clauses 133-142, wherein the inlet valve comprises a cylindrical valve body and the plurality of leaflets are coupled to the valve body.

144. The method of clause 143, wherein the conduit has a first inner diameter and the valve body has a second inner diameter, the second inner diameter being at least between about 70% and about 98% of the first inner diameter.

145. The method of any one of clauses 133-144, wherein cyclically operating the volume displacement member comprises cyclically operating the volume displacement member at a frequency of at least about 500 beats per minute.

146. The method of clause 145, wherein cyclically operating the volume displacement member comprises cyclically operating the volume displacement member at a frequency of between about 1000 beats per minute and about 5000 beats per minute.

147. The method of any one of clauses 133-146, wherein positioning the blood pump device comprises collapsing the blood pump device in a lumen of a delivery catheter, and inserting the delivery catheter in a blood vessel.

148. The method of clause 147, wherein the lumen of the delivery catheter is no more than about 6 mm in diameter.

149. The method of claim 147 or 148, wherein positioning the blood pump device further comprises deploying the blood pump device from the lumen of the delivery catheter at a pumping location in a heart chamber or blood vessel.

150. The method of clause 149, wherein the inlet valve is configured to self-expand from a collapsed valve configuration in the lumen of the delivery catheter to an expanded valve configuration as the blood pump device is deployed.

151. The method of clause 150, wherein the inlet valve in the expanded valve configuration has an expanded diameter that is between about 1.2 and about 6 times the diameter of the lumen of the delivery catheter.

152. The method of any one of clauses 133-151, wherein the valve repeatedly cycles between the open state and the closed state at a frequency of between about 500 beats per minute and about 5000 beats per minute.

153. The method of clause 152, wherein the valve is configured to be cyclically operated at a frequency of between about 1000 beats per minute and about 5000 beats per minute.

154. The method of any one of clauses 133-153, wherein during the expansion phase, the inlet valve maintains the closed state under a pressure differential of up to at least about 500 mmHg from the downstream side to the upstream side, without allowing blood leakage from the downstream side to the upstream side.

155. The method of clause 154, wherein the valve maintains the closed state under a pressure differential of up to at least about 700 mmHg from the downstream side to the upstream side, without allowing blood leakage from the downstream side to the upstream side

156. A valve arrangement for a blood pump device, the valve arrangement comprising:
a valve comprising a plurality of leaflets, an upstream side, and a downstream side, wherein the valve is operable in a valve cycle including:
   an open state in which the leaflets permit fluid flow between the upstream side and the downstream side; and
   a closed state in which the leaflets substantially prevent fluid flow from the downstream side to the upstream side,
wherein the valve is configured to transition from the closed state to the open state over no more than about 20% of the duration of the valve cycle.

157. The valve arrangement of clause 156, wherein the valve is configured to maintain the closed state under a pressure differential of up to at least about 500 mmHg from the downstream side to the upstream side, without allowing fluid leakage from the downstream side to the upstream side.

158. The valve arrangement of clauses 156 or 157, wherein the valve is configured to maintain the closed state under a pressure differential of up to at least about 700 mmHg from the downstream side to the upstream side, without allowing fluid leakage from the downstream side to the upstream side.

159. The valve arrangement of any one of clauses 156-158, wherein the valve is configured to be cyclically operated between the open state and the closed state at a frequency of at least about 500 beats per minute.

160. The valve arrangement of clause 159, wherein the valve is configured to be cyclically operated at a frequency of between about 1000 beats per minute and about 5000 beats per minute.

161. The valve arrangement of any one of clauses 156-160, wherein at least one leaflet comprises a base region comprising a first bending stiffness and an edge region comprising a second bending stiffness, wherein the second bending stiffness is lower than the first bending stiffness.

162. The valve arrangement of any one of clauses 156-161, wherein the base region comprises a first leaflet thickness and the edge region comprises a second leaflet thickness, wherein the first leaflet thickness is less than the second leaflet thickness.

163. The valve arrangement of any one of clauses 156-162, wherein the base region comprises a first material, and the edge region comprises a second material, wherein the second material has a greater durometer than the first material.

164. The valve arrangement of any one of clauses 156-163, wherein at least one leaflet comprises multiple layers of material.

165. The valve arrangement of any one of clauses 156-164, wherein the inlet valve has an outer diameter of about 25 mm or less.

166. A blood pump device comprising:
a conduit having an inflow region and an outflow region;
a volume displacement member arranged in the conduit; and
the valve arrangement of any one of clauses 156-165, wherein the valve arrangement is positioned in the inflow region of the conduit.

167. The blood pump device of clause 166, wherein the conduit comprises an expandable support and at least one membrane covering at least a portion of the expandable support.

168. The blood pump device of clause 167, wherein the at least one membrane covers an inner surface or outer surface of the expandable support.

169. The blood pump device of clause 167 or 168, wherein at least one of the plurality of leaflets is joined to the at least one membrane.

170. The blood pump device of clause 168 or 169, wherein the at least one membrane comprises an inner membrane covering the inner surface of the expandable support and an outer membrane covering the outer surface of the expandable support, and at least one of the plurality of leaflets is joined to the inner membrane or the outer membrane, or both.

171. The blood pump device of any one of clauses 166-170, further comprising a tubular member extending longitudinally through the conduit and between the leaflets of the valve.

172. The blood pump device of clause 171, wherein the tubular member is configured to slidably receive a guidewire.

173. The blood pump device of clause 171 or 172, wherein the leaflets are configured to coapt with an outer surface of the tubular member.

174. The blood pump device of clause 173, wherein at least one leaflet has an inner edge portion complementary with a cross-sectional profile of the tubular member.

175. The blood pump device of any one of clauses 171-174, wherein the tubular member is configured to support the leaflets to substantially inhibit prolapsing of the leaflets.

176. The blood pump device of any one of clauses 171-175, wherein the tubular member extends through an interior of the volume displacement member.

177. The blood pump device of any one of clauses 166-176, wherein the valve in its open state has a valve orifice area between about 15% and about 90% of a maximum cross-sectional area of the conduit, as measured at a location of the inlet valve.

178. The blood pump device of any one of clauses 166-177, wherein the valve comprises a cylindrical valve body and the plurality of leaflets are coupled to the valve body.

179. The blood pump device of clause 178, wherein the conduit has a first inner diameter and the valve body has a second inner diameter, the second inner diameter being at least between about 70% and about 98% of the first inner diameter.

180. The blood pump device of any one of clauses 166-179, wherein the blood pump device is collapsible into a lumen of a delivery catheter.

181. The blood pump device of clause 180, wherein the lumen of the delivery catheter is no more than about 6 mm in diameter.

182. The blood pump device of clause 181, wherein the valve is configured to self-expand from a collapsed valve configuration to an expanded valve configuration.

183. The blood pump device of clause 182, wherein the valve in the expanded valve configuration has an expanded diameter that is between about 1.2 and about 6 times the diameter of the lumen of the delivery catheter.

184. A blood pump device comprising:
a pump body comprising:
   a conduit having an inflow region and an outflow region, wherein the conduit comprises an expandable support and a membrane covering at least a portion of the expandable support;
   a balloon arranged in the conduit and cyclically operable between an expansion phase and a contraction phase; and
   an inlet valve arranged in the inflow region and comprising a plurality of leaflets,
   wherein the inlet valve transitions between an open state during the contraction phase to allow blood flow into the conduit, and a closed state during the expansion phase in which the valve substantially prevents blood flow out of the conduit through the inflow region,
   wherein each leaflet comprises a base region comprising a first bending stiffness and an edge region comprising a second bending stiffness lower than the first bending stiffness, and
   wherein each leaflet is joined to the membrane of the conduit;
a guidewire extending through the inlet valve; and
a tubular member configured to slidably receive the guidewire, wherein the tubular member extends through the inlet valve and is configured to support the leaflets to substantially inhibit prolapsing of the leaflets.

185. The blood pump device of clause 184, wherein the inlet valve is configured to maintain the closed state under a pressure differential of up to at least about 500 mmHg from a downstream side of the valve to an upstream side of the valve, without allowing fluid leakage from the downstream side to the upstream side.

186. The blood pump device of clause 185, wherein the valve is configured to maintain the closed state under a pressure differential of up to at least about 700 mmHg from the downstream side to the upstream side, without allowing fluid leakage from the downstream side to the upstream side.

187. The blood pump device of any one of clauses 184-186, wherein the valve is configured to be cyclically operated between the open state and the closed state at a frequency of at least about 500 beats per minute.

188. The blood pump device of any one of clauses 184-187, wherein the valve is configured to be cyclically operated at a frequency of between about 1000 beats per minute and about 5000 beats per minute.

189. The blood pump device of any one of clauses 184-188, wherein the valve is cyclically operable in a valve cycle comprising the open state and the closed state, wherein the valve is configured to transition from the closed state to the open state over no more than about 20% of the duration of the valve cycle.

190. The blood pump device of any one of clauses 184-189, wherein the valve in its open state has a valve orifice area between about 15% and about 90% of a maximum cross-sectional area of the conduit, as measured at a location of the inlet valve.

191. The blood pump device of any one of clauses 184-190, wherein the blood pump device is collapsible into a lumen of a delivery catheter.

192. The blood pump device of clause 191, wherein the lumen of the delivery catheter is no more than about 6 mm in diameter.

193. The blood pump device of clause 192, wherein the valve is configured to self-expand from a collapsed valve configuration to an expanded valve configuration.

194. The blood pump device of clause 193, wherein the valve in the expanded valve configuration has an expanded diameter that is at least between about 1.2 and about 6 times the diameter of the lumen of the delivery catheter.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is an illustrative schematic of an example circulatory assist device, in accordance with the present technology.

FIG. 1B is an illustrative schematic of an example circulatory assist device positioned in a patient, in accordance with the present technology.

FIGS. 2A-2E are illustrative schematics of an example circulatory assist device during operation, in accordance with the present technology.

FIGS. 3A-3C are illustrative schematics of cross-sectional views of various example supports in a circulatory assist device, in accordance with the present technology.

FIGS. 4A and 4B are side and perspective views, respectively, of an example circulatory assist device, in accordance with the present technology.

FIG. 5 depicts an example tricuspid inlet valve for a circulatory assist device, in accordance with the present technology.

FIGS. 6A-6D are illustrative schematics of an example tricuspid inlet valve, in accordance with the present technology.

FIGS. 6E and 6F are illustrative schematics of an example tricuspid inlet valve, in accordance with the present technology.

FIG. 6G is an illustrative schematic of leaflet of an inlet valve having multiple regions of varying bending stiffness, in accordance with the present technology.

FIGS. 6H-6K are illustrative schematics of example reinforcement members for an inlet valve, in accordance with the present technology.

FIGS. 6L-6N are illustrative schematics of cross-sectional views of various example reinforcement members for an inlet valve, in accordance with the present technology.

FIG. 6O is an illustrative schematic of an example circulatory assist device, in accordance with the present technology.

FIG. 7 is an illustrative schematic of an example circulatory assist device, in accordance with the present technology.

FIG. 8 is an illustrative schematic of an example circulatory assist device, in accordance with the present technology.

FIG. 8A is an illustrative schematic of an example circulatory assist device, in accordance with the present technology.

FIG. 9A is a side view of an example circulatory assist device, in accordance with the present technology. FIG. 9B is a cross-sectional view of the device of FIG. 9A, taken along the line 9B:9B.

FIG. 10A is a side view of an example circulatory assist device, in accordance with the present technology. FIG. 10B is a cross-sectional view of the device of FIG. 10A, taken along the line 10B: 10B.

FIGS. 11A and 11B are illustrative schematics of an example circulatory assist device in accordance with the present technology, with an inlet valve in a closed state and in an open state, respectively.

FIG. 12 is an illustrative schematic of an example circulatory assist device, in accordance with the present technology.

FIGS. 13A and 13B are illustrative schematics of an example circulatory assist device in accordance with the present technology, in a radially expanded configuration and a low profile configuration, respectively.

FIG. 14 is an illustrative schematic of a cross-section of an example coupling arrangement between a support and a catheter in a circulatory assist device, in accordance with the present technology.

FIG. 15 is an illustrative schematic of a portion of a proximal end of an example support for use in a coupling arrangement between the support and a catheter in a circulatory assist device, in accordance with the present technology.

FIGS. 16A and 16B are perspective and partial disassembled views, respectively, of an example coupling arrangement between a support and a catheter in a circulatory assist device, in accordance with the present technology.

FIGS. 16C and 16D are perspective and partial disassembled views, respectively, of an example coupling arrangement between a support and a catheter in a circulatory assist device, in accordance with the present technology.

FIGS. 17A and 17B are side views of an example coupling arrangement between a support and a catheter in a circulatory assist device, in accordance with the present technology.

FIG. 18 is a side view of an example coupling arrangement between a support and a catheter in a circulatory assist device, in accordance with the present technology.

FIGS. 19A and 19B are assembled and disassembled views, respectively, of an example coupling arrangement between a support and a catheter in a circulatory assist device, in accordance with the present technology.

FIGS. 20A and 20B are illustrative schematics of a support and a catheter, respectively, in an example coupling arrangement between the support and the catheter in a circulatory assist device, in accordance with the present technology.

FIGS. 21A and 21B are side views of an example coupling arrangement between a support and a catheter in a circulatory assist device, in accordance with the present technology.

FIGS. 22A and 22B are a perspective view of a catheter, and a perspective view of a catheter with locking pins, respectively, in an example coupling arrangement between a support and a catheter in a circulatory assist device, in accordance with the present technology.

FIGS. 23-25 are cross-sectional views of example catheters 110 with various reinforcement features.

FIGS. 26A and 26B are illustrative schematics of an example circulatory assist device in accordance with the present technology, in a low profile configuration and a radially expanded configuration, respectively.

FIGS. 27A and 27B are illustrative schematics of an example circulatory assist device in accordance with the present technology, in a low profile configuration and a radially expanded configuration, respectively.

FIGS. 28A and 28B are illustrative schematics of an example circulatory assist device in accordance with the present technology, in a low profile configuration and a radially expanded configuration, respectively.

FIGS. 29A and 29B are illustrative schematics of an example circulatory assist device in accordance with the present technology, in a low profile configuration and a radially expanded configuration, respectively.

FIG. 30 is an illustrative schematic of a portion of an example circulatory assist device without a pigtail connector, in accordance with the present technology.

FIG. 31A is an illustrative schematic of an example circulatory assist device. FIG. 31B is a detailed view of a portion of FIG. 31A, including an illustrative partial schematic of a joining arrangement between an inlet valve and conduit of an example circulatory assist device.

### DETAILED DESCRIPTION

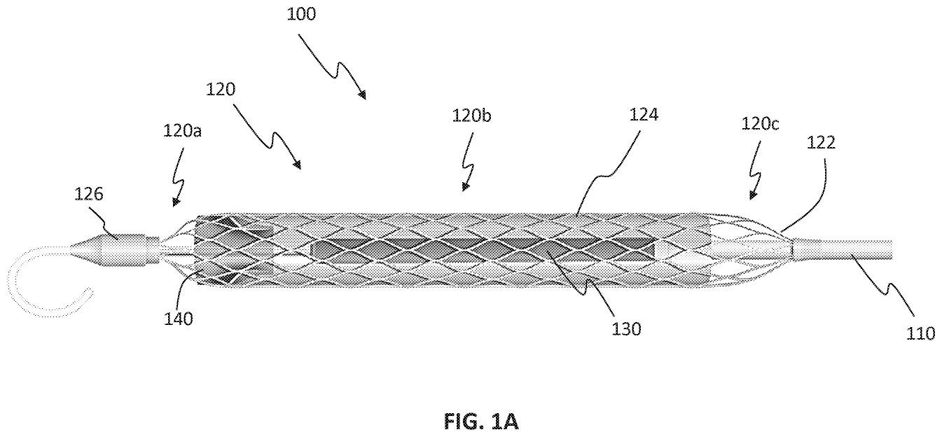

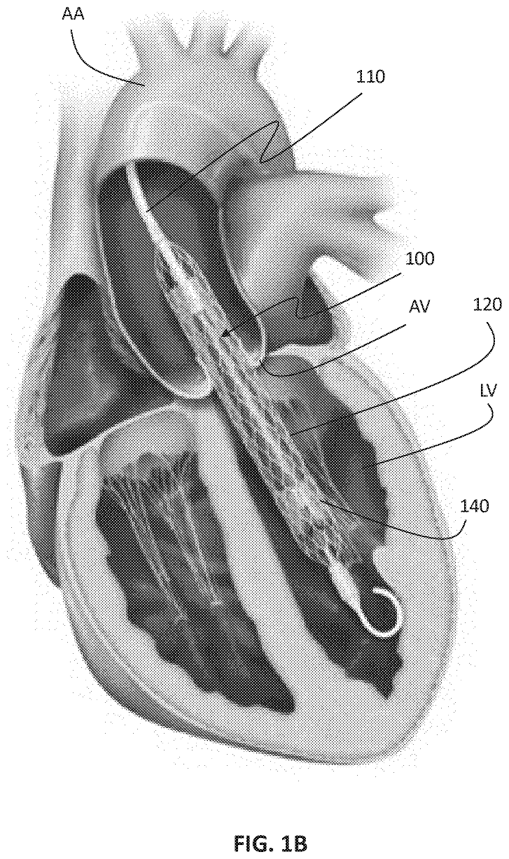

The present technology relates to circulatory assist systems and methods. Some aspects of the present technology, for example, are directed to cardiac assist devices and methods. Such devices can, for example, be delivered percutaneously into a cardiovascular lumen and are capable of pumping blood at flows high enough to support patients in cardiogenic shock, acute myocardial infarction, acute heart failure or during high-risk percutaneous coronary interventions, or other situations requiring hemodynamic support with reduced levels of hemolysis. Specific details of several aspects of the technology are described below with reference to FIGS. 1A-31B.

As used herein, the terms “proximal” and “distal” (and derivatives thereof) are used primarily within a frame of reference of a user placing a circulatory assist device within a patient, unless otherwise specified. For example, “proximal” primarily refers to a direction closer to the user, while “distal” primarily refers to a direction farther from the user.

The circulatory assist devices and systems of the present technology may be used to provide circulatory assistance (e.g., cardiac assistance) in a variety of procedures and to address a variety of patient conditions. For example, the circulatory assist devices and systems may be used for cardiac assist during high-risk percutaneous coronary interventions (PCI) including angioplasty and stenting. Furthermore, the circulatory assist devices and systems may be used to provide cardiac support for patients experiencing cardiogenic shock. Furthermore, the circulatory assist devices and systems may be used to provide cardiac support for patients experiencing acute myocardial infarction. Generally, for such procedures, the heart assist devices will be configured for placement at least partially in the left ventricle. However, placement at various other cardiovascular lumen sites is also possible, including at least partially in the ascending or descending aorta, the aortic arch, the right atrium, right ventricle, or pulmonary artery.

### I. Circulatory Assist Systems

In some variations, a circulatory assist system includes a circulatory assist device that is positionable in a patient (e.g., in a cardiovascular lumen, such as a blood vessel and/or heart chamber). For example, the circulatory assist device can function as a percutaneous ventricular assist device (pVADs), a transvalvular pVAD, or an intra-vascular and intra-ventricular blood pump, though other uses of the circulatory assist device are contemplated.

In some variations, the circulatory assist device (e.g., blood pump) can include a pump body having a conduit, at least one inlet valve, and a volume displacement member arranged in the conduit. The conduit may have an inlet, an outlet, and a longitudinal flow axis extending between the inlet and the outlet. The inlet valve may be configured to receive a fluid (e.g., patient body fluid such as blood) along the flow axis. The inlet valve may be configured to receive a fluid (e.g., patient body fluid such as blood) along the flow axis. Furthermore, in some variations, the portion of the conduit between the volume displacement member and an outlet of the conduit may be valveless (e.g., the circulatory assist device may include only one or more inlet valves). The fluid column traveling in an axial direction in the conduit may have particular advantages, as described below. The volume displacement member may be operable in an expansion phase and a contraction phase, and the pump body may be configured to convey fluid through the outlet during both at least a part of the expansion phase and at least a part of the contraction phase of the volume displacement member. In some variations, the pump body may be configured to substantially continuously convey fluid through the outlet during both at least a part of the expansion phase and at least a part of the contraction phase of the volume displacement member. Additionally or alternatively, in some variations the circulatory assist device may include other suitable pump device(s) configured to convey fluid through the outlet of the conduit.

For example, in some variations, as shown in FIG. 1A, a circulatory assist device 100 can include a pump body 120 configured to receive a fluid (e.g., blood) when placed in a cardiovascular lumen of a patient and convey the fluid back to the cardiovascular lumen for circulatory assistance. The pump body 120 may include a conduit with a distal inflow region 120a that conveys fluid into the pump body 120, a proximal outflow region 120c that conveys fluid out of the pump body 120, and an intermediate region 120b between the inflow region 120a and the outflow region 120c. In some variations, the intermediate region 120b is configured to retain received fluid until the fluid exits through the outflow region 120c. Generally, the pump body 120 (at least the intermediate region 120b, for example) may have an elongated shape (e.g., cylindrical or elongated with an elliptical cross-section), such as a tubular or pipe-like shape. However, in some variations the pump body 120 may have an elongated shape with a varying cross-section (e.g., the pump body 120 may be bulbous or hourglass-shaped) along the conduit length. The conduit may have a longitudinal flow axis extending between an inlet of the inflow region 120a and an outlet of the outflow region 120c. The flow axis may generally follow a longitudinal axis of the conduit of the pump body 120, which may be at least partially linear and/or at least partially curved. As such, the flow axis between the inlet and the outlet may be linear, may be curved, or may have one or more portions that are linear and one or more portions that are curved. Furthermore, the exact path of the flow axis may change dynamically depending on, for example, the shape of the pump body 120 during any given point in time during operation (e.g., a linear portion of the flow axis may become more curved, a curved portion of the flow axis may become more linear, the contour of a curved portion of the flow axis may change, etc.). For example, as the pump body 120 is operated, the exact relative positions of the inlet and the outlet of the conduit may change.

As further described below, the pump body 120 may further include an inlet valve 140 configured to receive a fluid through the inlet of the conduit along the flow axis, and a volume displacement member 130 (e.g., balloon) arranged in the conduit. The volume displacement member 130 may be operable in an expansion phase and a contraction phase. For example, in variations in which the volume displacement member 130 includes a balloon, the balloon may be inflated in the expansion phase, and deflated in the contraction phase. In operation, the pump body 120 may be configured to convey the received fluid through an outlet of the outflow region 120c during both at least a part of the expansion phase and at least a part of the contraction phase of the volume displacement member 130. In some variations, such conveyance of fluid through the outlet may be sustained at least partially due to created and maintained momentum of a fluid column along the flow axis of the conduit. Additional details of continued momentum of flow, and further example features for creating and maintaining momentum of fluid in the pump body 120, are described in U.S. Provisional Patent Application No. 63/516,792, which is incorporated in its entirety by this reference.

In some variations, the pump body 120 may be coupled to or otherwise arranged on a catheter 110, which can be used to position the pump body 120 in the patient and/or facilitate operation of the volume displacement member 130 in the expansion phase and the contraction phase. In some variations, the pump body 120 may be coupled to a distal portion of the catheter 110, while a proximal portion (not shown in FIG. 1A) of the catheter 110 may be outside the patient and coupled to a handle for facilitating placement of the pump body 120. Additionally or alternatively, the proximal portion of the catheter 110 may be coupled to a control system and/or actuator for controlling the volume displacement member 130 in the expansion phase and the contraction phase. For example, the catheter 110 may include one or more lumens for conveying a guidewire that is controlled external to the patient for positioning the pump body 120 in the patient. Additionally or alternatively, in some variations (e.g., in which the volume displacement member 130 includes a balloon), the catheter 110 may include one or more lumens for conveying a fluid (e.g., gas) for cyclically inflating the balloon.

As shown in FIG. 1B, in some variations, the circulatory assist device 100 may be configured for placement at least partially in the left ventricle (LV) and/or the ascending aorta (AA). For example, in some variations, the pump body 120 can be placed across the native aortic valve (AV) such that a first portion of the pump body 120 (including some or all of the distal inflow region) is in the left ventricle, and a second portion of the pump is in the ascending aorta. In some variations, the pump body 120 can be placed such that the inlet valve is located in the left ventricle, below the plane of the aortic valve. In some variations, the pump body 120 can be placed such that the inlet valve is located in the aorta (e.g., ascending aorta) above the plane of the aortic valve, while a portion of the inflow region is in the left ventricle. Although the pump body 120 is shown in FIG. 1B as having approximately half of its length in the left ventricle and half of its length in the ascending aorta, it should be understood that placement of the pump body 120 may vary. For example, in some variations the pump body 120 can be placed such that less of its length (e.g., about 20%, 30%, 40% of the pump body length) is in the left ventricle than the length (e.g., about 80%, 70%, 60%) that is in the ascending aorta. In some instances, it may be beneficial to reduce the volume of the pump body 120 that is placed in the left ventricle, as this positioning limits the amount of potentially adverse interaction between the pump body 120 and the heart muscle and valves (e.g., reduce risk of obstruction of the mitral valve). However, in some variations any suitable portion of the pump body 120 can be placed in the left ventricle. For example, the pump body 120 can be placed such that more of its length (e.g., about 80%, 70%, 60%) of the pump body length is in the left ventricle than the length (e.g., about 20%, 30%, 40%) that is in the ascending aorta. In some variations, the pump body 120 is placed such that a portion of its intermediate region 120b (such as somewhere along the conduit length including the volume displacement member 130) crosses the aortic valve (e.g., is located at the plane of the aortic valve). In some variations, the pump body 120 is placed such that a portion of its outflow region 120c crosses the aortic valve (e.g., is located at the plane of the aortic valve).

As described above, the circulatory assist device 100 is characterized by axial flow between the inlet and the outlet of the conduit. In other words, in some variations, fluid pumped by the circulatory assist device 100 travels from the inlet to the outlet substantially entirely or predominantly axially along (e.g., aligned with) the flow axis of the conduit. In some variations, the fluid flow in the conduit has limited to no radial flow component., and/or limited to no circumferential flow component. The flow axis of the conduit may be substantially coincident with a longitudinal axis of the conduit, for example, though it should be understood that axial flow includes both flow of fluid coincident with the longitudinal axis and flow of fluid generally parallel to the longitudinal axis. The circulatory assist device 100 with axial flow may have a number of advantages. For example, because forces acting on the fluid within the pump body are generally oriented in the same direction, the fluid travels in a linear path through the circulatory assist device 100 and experiences less turbulence, thereby resulting in less disturbance in components of the fluid itself (e.g., less hemolysis in blood pumped by the circulatory assist device 100). Additionally, since flow occurs all in the same general axial direction (e.g., with little to no radial flow component), the kinetic behavior of the pump body (e.g., expansion and contraction of the volume displacement member, such as inflation and deflation of a balloon) can be more streamlined and energy efficient.

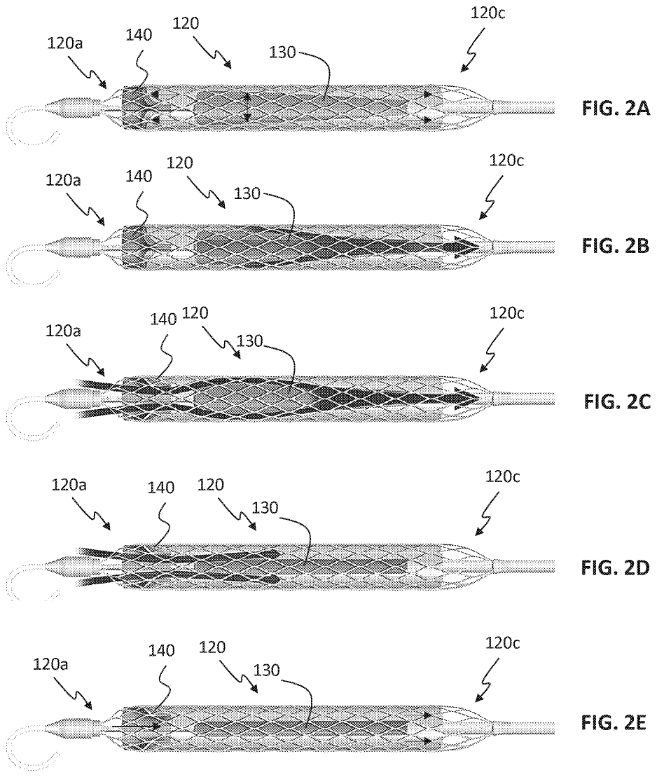

FIGS. 2A-2E illustrate various phases of operation in which fluid may be allowed to exit the conduit of the pump body via maintained momentum during the expansion phase and at least a part of the contraction phase of a volume displacement member 130. Although the volume displacement member 130 is primarily shown and described below as a balloon, it should be understood that the same principles of operation apply with respect to other variations of circulatory assist devices that include different kinds of volume displacement members 130.

FIG. 2A illustrates a pump body 120 that has received fluid (e.g., blood) through the inlet valve 140, and has a volume displacement member 130 being inflated to expand within the pump body 120. As the volume displacement member 130 inflates, it displaces surrounding fluid, thereby pushing fluid both distally toward the inflow region 120a and proximally toward the outflow region 120c. Fluidic pressure causes the inlet valve 140 to close, while also urging fluid to exit the pump body 120. As shown in FIG. 2B, when the inlet valve 140 is fully closed, all of the fluid volume in the pump body 120 exits through the outlet of the outflow region 120c.

Inflation of the volume displacement member 130 also helps generate momentum of the fluid column traveling toward the outflow region 120c in the pump body 120. FIG. 2C illustrates when the volume displacement member 130 is at an end portion of the expansion phase, and the volume displacement member 130 is inflated to a maximum volume. At this stage of operation, the fluid mass in the pump body 120 has momentum toward the outflow region 120c to exit the pump body 120 through the outlet, and such movement of the fluid mass results in a negative pressure within the pump body 120. Under such momentum and negative pressure within the pump body 120, the inlet valve 140 opens and additional fluid is pulled into the pump body 120 through the open inlet valve 140 in the axial flow direction, as shown in FIG. 2C.

As shown in FIG. 2D, when the volume displacement member 130 enters its contraction phase and begins to deflate, the fluid momentum continues, and additional fluid is pulled into the pump body 120 in the axial flow direction through the open inlet valve 140. In some instances, the amount of fluid momentum may decrease at this stage if additional fluid is also pulled into the pump body 120 through the outflow region 120c. However, in these instances, the momentum (and volume) of fluid pulled through the inflow region 120a is greater than that of fluid pulled distally through the outflow region 120c. Accordingly, fluid momentum may slow, but still continues in the direction from the inflow region 120a toward the outflow region 120c, thereby drawing in additional fluid into the pump body 120 for further pumping.

FIG. 2E illustrates when the volume displacement member 130 is at an end portion of the contraction phase, and the volume displacement member 130 is deflated to a minimum volume. At this stage of operation, the fluid mass continues to have momentum in the proximal direction toward the outflow region 120c, and fluid continues to exit the pump body 120 through the outlet of the conduit. Following the contraction phase, the volume displacement member 130 returns to its expansion phase, and the above-described cycle of expansion and contraction (with continued momentum and fluid conveyance through the conduit outlet, as shown in FIGS. 2A-2E) may repeat. Further details regarding axial flow in the circulatory assist device 100 are described in U.S. Provisional Patent Application No. 63/516,792, which was incorporated by reference above.

Other aspects of the present technology may have additional benefits. For example, the pump body 120 can have an elongated shape that is well-suited for axial flow, which can be advantageous because increased length of the pump body 120 provides a longer landing zone that can be placed within and against leaflets of a valve (e.g., native leaflets of an aortic valve between the left ventricle and the ascending aorta). Accordingly, an elongated circulatory assist device 100 can be delivered to a transvalvular position with greater ease, as less precision is required for the circulatory assist device 100 to be placed in a suitable position across a valve (e.g., aortic valve).

Further details of the circulatory assist device 100, and methods of treatment using the circulatory assist device 100, are described below.

### A. Pump Body

As described above, the circulatory assist device 100 may include a pump body 120 that functions to receive a fluid (e.g., blood) when placed in a cardiovascular lumen and pump the fluid to provide circulatory assistance. Generally, the pump body 120 may be sized to fit the intended anatomy without causing obstruction of fluid flow. For example, in some variations, the pump body 120 in an expanded state may have a diameter of about 15 mm or less (e.g., at least in the outflow region 120c). In some variations, the pump body 120 in the expanded state may be configured (e.g., sized and shaped) to extend through a native cardiac valve such as an aortic valve. In some variations, the pump body 120 may be flexible (or pre-formed with a suitable contour or other shape) to conform to surrounding anatomy and avoid tissue trauma.

In some variations, as shown in FIG. 1A, the pump body 120 may include a conduit with an expandable support 122 and at least one fluid impermeable membrane 124 adjacent to the expandable support. The pump body 120, for example by virtue of the geometric and/or material properties of the support 122 and/or the membrane 124, may be substantially non-compliant so as to resist deformation during the expansion and contraction cycles of the volume displacement member 130. Furthermore, the conduit may have a circular, ellipsoidal, or other suitable curved wall, so as to help the conduit (and the overall pump body 120) be more resistant to failure in response to internal pressure (i.e., positive and/or negative pressure). Additionally, the curved wall of the conduit may help facilitate suitable clearance between the outer surface of the volume displacement member 130 and the conduit, as further described herein.

The support 122 functions at least in part to provide structural support to the pump body 120. For example, the support 122 may help the conduit to be resistant against diametrical expansion in response to increased pressure when filled with blood and during expansion of the volume displacement member. Such non-distensibility allows spacing to be maintained between the pump body 120 and the ventricular wall to minimize trauma to heart tissue and also increases pump efficiency. Additionally, the support 122 may help the conduit be resistant against collapsing in response to decreased pressure (e.g., during contraction of the volume displacement member). In some variations, the support 122 may be configured to be collapsible or crimpable into a lower profile transport state during delivery to the target placement location (e.g., left ventricle and/or ascending aorta), and/or when subject to sufficient external forces to allow for endovascular delivery and retrieval. The support 122 may further be configured to expand into a deployed state, such as by self-expansion and/or expansion with another device (e.g., balloon-expandable). In some variations, the support 122 may include a frame or skeleton of a resilient metal such as nickel-titanium alloy, cobalt-chrome, chromoly steel, or stainless steel, etc., or a suitable polymeric material such as nylon. The support 122 may, for example, include woven wires, mesh, a basket, laser-cut material, or a monolithic tube having an arrangement of openings, slits, or cells which allow expansion in at least one dimension from the transport state to the deployed state. For example, the support 122 can include a plurality of struts or cells arranged in a radially expandable geometry. The support 122 can include a single continuous body, or can include multiple bodies coupled together (e.g., nested mesh tube structures with overlapping walls).

In some variations, at least a portion of the support 122 may have a generally tubular shape. For example, at least the portion of the support 122 forming the intermediate region 120b can be tubular. In some variations, at least a portion of the support 122, such as at least the portion of the support 122 forming the intermediate region 120b, can be tubular with a constant cross-sectional shape (e.g., cylindrical) or a varying cross-sectional shape along its length (e.g., bulbous, hourglass-shaped). The support 122 can have at least one closed end. For example, as shown in FIG. 1A, in some variations the distal end of a generally tubular support 122 (e.g., the distal end of the inflow region 120a) may be closed by a connector 126 (e.g., crimp, tube, etc. that may connect free ends of wires forming the support 122) or other suitable mechanical fastener(s), and/or joined in any other suitable manner such as welding, etc. Furthermore, in some variations the proximal end of the support 122 (e.g., the proximal end of the outflow region 120c) may be closed through integral formation, such as by virtue of the pattern of weaving, mesh, laser-cutting, etc. It should be understood that in some variations, either or both of the distal end of the support 122 and the proximal end of the support 122 may additionally or alternatively be closed through integral formation, mechanical fastener(s), welding, and/or the like. Furthermore, in some variations, the distal end of the support 122 and/or the proximal end of the support 122 may instead be open.

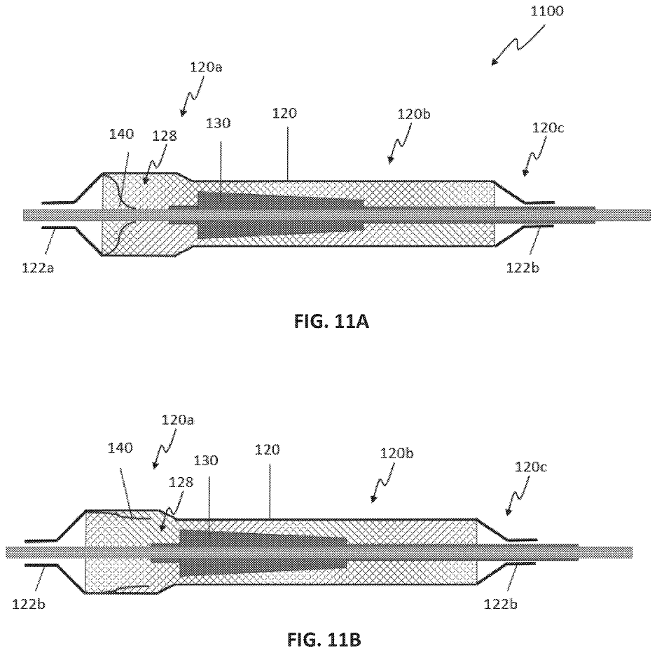

In some variations, the inflow region 120a of the support 122 may have a different profile (e.g., diameter) than other regions of the support 122. For example, the inflow region 120a of the support 122 may have a different profile than the intermediate region 120b and/or outflow region 120c of the support 122. In some variations, as shown in FIGS. 11A and 11B, a portion of the support 122 surrounding the operating region of the valve 140 may be enlarged relative to the intermediate region 120b, so as to provide a recessed volume 128 within the support 112. In these variations, leaflets of a multi-leaflet valve 140 (or other moving components of the valve) may expand into the recessed volume 128 when the valve 140 is in the open state (FIG. 11B). By allowing the leaflets of the valve 140 to fall or settle into the recessed volume 128 when the valve 140 is in the open state, the leaflets can be prevented from limiting the main flow diameter of the conduit in the pump body 120. Accordingly, in some variations, the main flow diameter of the conduit in the pump body 120 can be consistent throughout the length of the conduit. In some variations, the wall of the expanded support 122 can have a stepped profile that transitions from a larger diameter in the inflow region 120a including the recessed volume 128, to a smaller diameter in the intermediate region 120b. In some variations, the difference between the larger diameter in the inflow region 120a and the smaller diameter in the intermediate region 120b may be generally equal to at least twice the thickness of the valve leaflets, which can allow the valve leaflets to fully sit in the recessed volume 128 when the valve 140 is in the open state, without limiting the flow passageway.

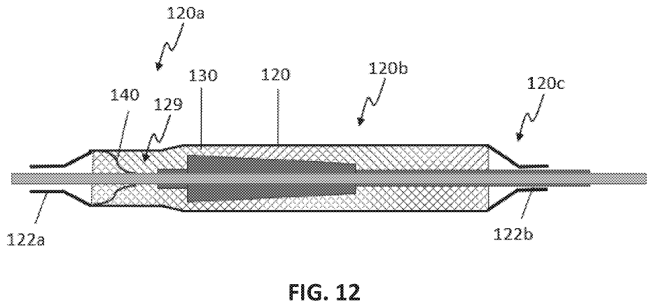

In some variations, as shown in FIG. 12, a portion of the support 122 surrounding the operating region of the valve 140 may have a reduced diameter relative to the intermediate region 120b, so as to reduce the amount of material of the support 122 around the valve 140 that needs to be crimped when the pump body 120 is collapsed to a low-profile configuration. This reduced volume of support 122 material may help maintain the low profile of the crimped pump body 120 by compensating for the added volume of the valve 140 material. For example, at least a portion of the inflow region 120a may include a necked region 129 that is narrower than the intermediate region 120b when the support 122 is expanded. In some embodiments, the crimped profile of the pump body 120 in the necked region 129 (including combined material of the valve 140, the support 122, and the membrane 124) may be generally equal to (or less than) the crimped profile of the pump body 120 in the intermediate region 120b (including combined material of the support 122, the membrane 124, and the balloon 130). In some variations, the difference between the smaller diameter of the necked region 129 and the larger diameter of the intermediate region 120b may be generally equal to the difference between the diameter of the valve 140 when crimped and the diameter of the balloon when crimped.

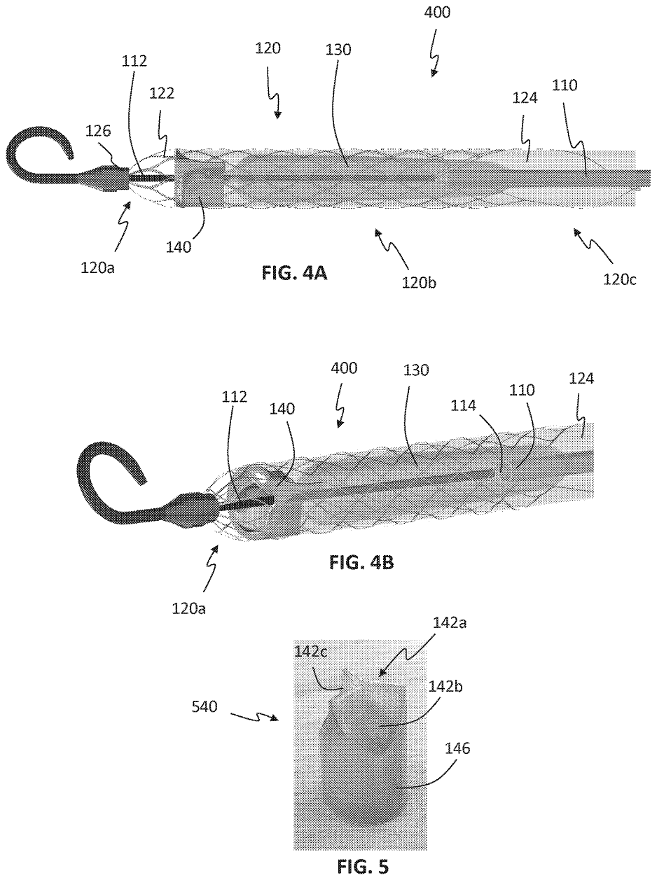

In some variations, the pump body 120 may include one or more features to aid repositioning or retrieval of the pump body 120 from the patient (e.g., after circulatory assistance is no longer needed, or if the pump body 120 is to be swapped with another circulatory assist device). Additionally or alternatively, the distal end of the pump body 120 may include one or more atraumatic features to help reduce or avoid tissue trauma in the event that the distal end of the pump body 120 abuts tissue (e.g., left ventricle wall). For example, as shown in FIGS. 4A and 4B, a connector 126 coupled to or integrally formed with a distal end of the pump body 120 may include a curved pigtail structure at the distalmost end of the connector.

In some variations, the pump body 120 may include at least one fluid impermeable membrane 124 adjacent to a surface of the support 122. The membrane 124 may extend along at least a portion of the length of pump body 120. For example, as shown in FIG. 1A, the membrane 124 may extend along at least the intermediate region 120b of the pump body 120. The membrane 124 may further extend along at least a portion of the inflow region 120a and at least a portion of the outflow region 120c. In some variations, at least a portion of the inflow region 120a and/or the outflow region 120c may remain uncovered by the membrane 124 so as to permit passage of fluid through the support 122 in and/or out of the conduit (e.g., through open and uncovered cells of the support 122). The membrane 124 may include one continuous layer of material, or may include multiple segments of material that are coupled to one another (e.g., radial or longitudinal strips sealed to one another, such as by heat welding).

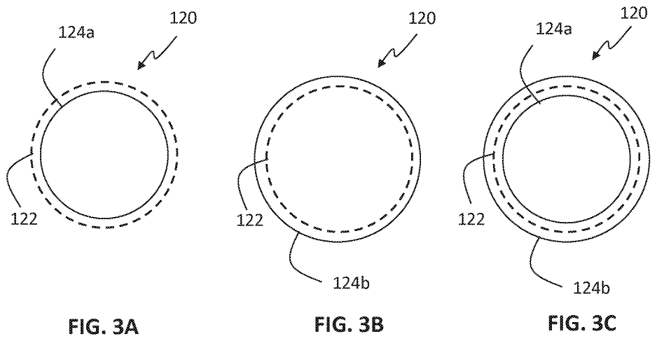

The pump body 120 may include at least one membrane 124 adjacent to an inner surface and/or an outer surface of the support 122. In some variations, one or more membranes 124 may be adjacent to an inner surface and/or an outer surface of the support 122. That is, the one or more membranes 124 may include an inner membrane 124a and/or an outer membrane 124b. For example, as shown in the cross-sectional view depicted in FIG. 3A, the pump body 120 may include an inner membrane 124a coupled to an inner surface of the support 122.

As another example, as shown in the cross-sectional view depicted in FIG. 3B, the pump body 120 may include an outer membrane 124b adjacent to an outer surface of the support 122. In this example of FIG. 3B, the outer membrane 124b may be coupled to the outer surface of the support 122, or may be unattached from the outer surface of the support 122. In variations in which the outer membrane 124b is not attached to the outer surface of the support 122, the outer membrane 124b may overlie the outer surface of the support 122 such that the outer membrane 124b is configured to expand in tandem with the support 122 when the support 122 is expanded. In these variations similar to the example of FIG. 3B, the outer membrane 124b may help contain radially outward pressure in the pump body 120, while the support 122 may help prevent collapse of the pump body 120 by providing outward support against negative (e.g., inward) pressure.

As yet another example, as shown in the cross-sectional view depicted in FIG. 3C, the pump body 120 may include both an inner membrane 124a and an outer membrane 124b, such that at least a portion of the support 122 is sandwiched between membrane layers. In some variations, the inner membrane 124a may be coupled to an inner surface of the support 122 and/or the outer membrane 124b may be coupled to an outer surface of the support, although in some variations neither the inner membrane 124a nor the outer membrane 124b may be coupled to the support. Furthermore, the pump body 120 may include more than one layer of an inner membrane 124a (e.g., two or more inner membrane layers), and/or more than one layer of an outer membrane 124b (e.g., two or more outer membrane layers). In variations in which the pump body 120 includes both an inner membrane 124a and an outer membrane 124b, the inner membrane 124a and the outer membrane 124b may extend over the same portions of the support 122, or may extend over different portions of the support 122.

The inner membrane 124a and/or the outer membrane 124b may be coupled to the support in any suitable manner, including, for example, spray lamination, welding, bonding, electrospinning, and/or adhesive. Furthermore, in some variations, the support 122 may be at least partially embedded within a fluid impermeable membrane 124 (e.g., via overmolding or other suitable technique). In some variations, the one or more membranes 124 may be coupled to the support 122 continuously along the inner and/or outer surfaces of the support 122. However, in some variations, at least some of the one or more membranes 124 may be coupled to the support 122 at only a portion of the inner and/or outer surfaces of the support 122, such as only along certain selected axial locations of the support 122 and/or certain selected radial locations around the support 122).

In some variations, the material of the one or more membranes 124 may be flexible and durable, such as nylon or polyurethane with high durometer values. This can be achieved by using a polymer with high tensile modulus. For example, the membrane can comprise a TPU such as pellathane or tecothane. In one example, the membrane can include tecothane in a durometer of approximately 72 D and have a wall thickness of between about 40 μm and about 300 μm, which may accommodate the stress placed on the conduit during operation of the circulatory assist device 100, without undergoing plastic deformation (e.g., resist stretching when the conduit is pressurized, such as when the conduit contains fluid and the volume displacement member is expanded). As another example, the membrane can include an inner membrane and an outer membrane, each including tecothane in a durometer of approximately 72 D with a wall thickness of between about 20 μm and about 150 μm, or between about 20 μm and about 100 μm, where the inner and outer membranes may have equal wall thicknesses, or may have different wall thicknesses. For example, in some variations the inner membrane may include 72 D tecothane and have a wall thickness of about 40 μm, and the outer membrane may include 72 D tecothane and have a wall thickness of about 60 μm, or vice versa. In some variations, the one or more membranes 124 may include an inelastic (e.g., non-compliant) material. For example, an inelastic material for the one or more membranes 124 may be suitable in variations in which the membrane(s) 124 are coupled to the support 122 at only a portion of the inner and/or outer surfaces of the support 122.

In some variations, the pump body 120 may also include one or more circumferential fibers of a material with a high tensile strength, which function to further limit the distensibility of the support 122 beyond its desired size, while still allowing the pump body 120 to be radially collapsed into a transport state (e.g., for insertion and removal). Such circumferential fibers may be arranged, for example, circumferentially around at various axial locations along the intermediate region 120b of the pump body 120. The circumferential fibers can include any suitable material such as Kevlar, spectra, carbon nanotubes, and/or other such materials that are attached, embedded within, or woven into the support 122 and/or membrane 124.

### B. Volume Displacement Member

As described herein, the circulatory assist device 100 may include at least one volume displacement member 130. The volume displacement member 130 functions to help create and maintain momentum in the fluid column through the conduit of the pump body 120, thereby drawing fluid into the conduit through the inlet valve 140 and enabling flow of fluid through the outlet of the conduit. Accordingly, in some variations, the inlet valve 140 may be configured to receive blood into the conduit and the pump body may be configured to convey the received blood through the outlet region, due to cyclical operation of the volume displacement member 130 between an expansion phase and a contraction phase.

The volume displacement member 130 may include any of various types of mechanisms capable of displacing a volume of fluid in a cyclical, repeating manner. In some variations, the volume displacement member 130 may include an inflatable balloon that can be inflated with a fluid to an expanded, high-volume state and deflated partially or completely to a contracted, low-volume state. In other variations, a piston, bellows, accordion-style expandable body, and/or other type of volume displacement member may be used. The volume displacement member is capable of moving cyclically between the contracted low-volume state in which it occupies a smaller portion of the conduit, to an expanded high-volume state, in which it occupies a substantially larger portion of the conduit, thus displacing blood therefrom. The volume displacement member 130 may be configured to cyclically move between these contracted and expanded states at a high frequency, such as at least about 300 beats per minute, at least about 500 beats per minute, at least about 1000 beats per minute, or at least about 1200 beats per minute, at least about 1500 beats per minute, at least about 2000 beats per minute, at least about 2500 beats per minute, at least about 3000 beats per minute (e.g., between about 1000 beats per minute and about 3000 beats per minute). In some variations, the frequency of the contraction/expansion cycle of the volume displacement member 130, in combination with the features (e.g., dimensions) of the rest of the pump body 120, is controlled such that the pump body is configured to convey fluid through the outlet with a flow rate of at least about 5 L/min.

When in the fully expanded state, the volume displacement member 130 may have a maximum diameter that is smaller than the inner diameter of the support 120, thereby allowing the outer surface of the expanded volume displacement member 130 to be spaced apart from the support 120, which provides clearance for fluid to move through the conduit between the inlet and the outlet even when the volume displacement member 130 is fully expanded. Such clearance may, in some instances, further function to help limit hemolysis during high frequency operation of the volume displacement member 130. For example, in some variations, when the volume displacement member 130 is fully expanded, a spacing of at least about 0.05 mm, or at least about 1.0 mm-5.0 mm (e.g., about 1.0 mm-3.0 mm) may be maintained between the volume displacement member 130 and an interior surface of the support 120.

As further described below with respect to the catheter 110, the volume displacement member 130 may be coupled to a shaft of the catheter 110. Additionally or alternatively, in some variations, the volume displacement member 130 may be coupled to the conduit of the pump body 120 (e.g., support 122 and/or membrane 124), which may help to anchor the volume displacement member 130 in a fixed position relative to the pump body 120, thereby minimizing movement of the volume displacement member 130 relative to the pump body 120 (other than from inflation) and reducing vibration of pump body 120.

In variations in which the volume displacement member 130 is a balloon, it may include a durable material such as polyurethane or nylon. The balloon may be formed of a single, thin wall of such material. For example, in one illustrative variation, the balloon may be made of pellethane 55D (or a material with similar mechanical properties), and have a wall thickness of about 10 μm-200 μm (e.g., about 20 μm). As shown in FIG. 1A, the balloon may have a generally elongated shape and extend longitudinally along a portion of the length of the pump body 120. For example, the balloon in its expanded state may have a generally ellipsoidal shape.

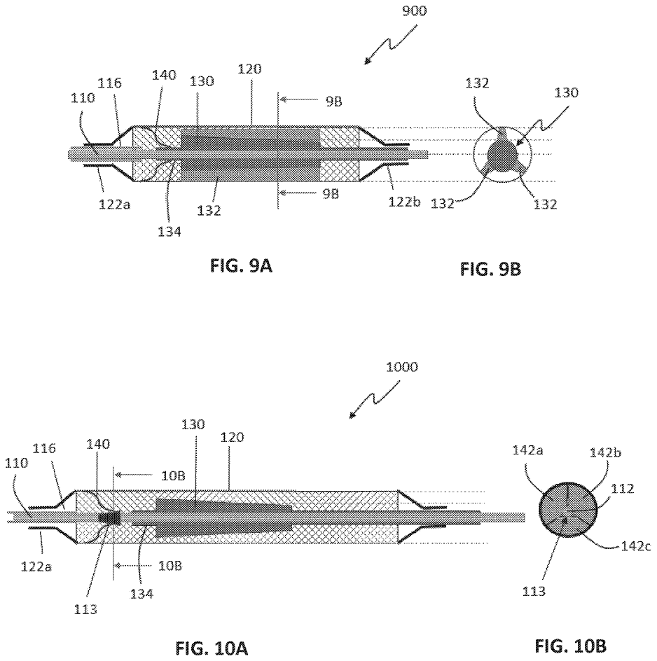

In some variations, the volume displacement member 130 may include one or more features to help maintain positioning (e.g., radial positioning) of the volume displacement member 130 within the pump body 120. For example, the volume displacement member 130 may include one or more radial positioning features to help keep the volume displacement member 130 located at a certain radial position within the pump body 120. For example, as shown in FIGS. 9A and 9B, the volume displacement member 130 may include a balloon with one or more radial projections 132 (e.g., ribs, rings, etc.) arranged around the circumference of the balloon. For example, the one or more radial projections 132 may extend longitudinally parallel to a longitudinal axis of the volume displacement member. As another example, the one or more radial projections 132 may extend circumferentially (e.g., sweep helically, wrap, etc.) around a longitudinal axis of the volume displacement member. The plurality (e.g., two, three, four, or more) radial projections 132 may be equally or unequally around the circumference of the balloon. In some variations, the radial projections may have generally equal radial lengths, so as to help keep the volume displacement member 130 radially centered within the pump body 120 (e.g., aligned with the longitudinal flow axis). In some variations, the radial projections may have different radial lengths, so as to radially offset or laterally displace the volume displacement member 130 relative to the center of the pump body 120 (e.g., radially offset from the longitudinal flow axis).

In some variations, the distance to which the radial projections 132 outwardly extend may be generally equal among the various radial projections 132. In these variations, when the balloon is inflated, the radial projections 132 may abut the inner surface of the conduit (e.g., support 122 or membrane 124) in a radially symmetric manner so as to help maintain the balloon centered within the pump body. In some variations, as shown in FIG. 9B, the cross-sectional profile of a radial projection 132 may be tapered in the radially outward direction, which, for example, help reduce the overall volume that the balloon occupies when the pump body 120 is radially crimped into a low-profile configuration.

In some variations, one or more radial projections 132 may extend longitudinally along the length of the balloon. For example, a radial projection 132 may extend longitudinally along the entire length of the balloon (or along the entire length of the inflatable portion of the balloon). As another example, a radial projection 132 may extend longitudinally along only an axial portion of the balloon. In some variations, a series of multiple radial projections 132 may be arranged intermittently along the length of the balloon (e.g., regularly or irregularly spaced apart by 2 mm, 3 mm, 4 mm, or 5 mm, etc.).

Additionally or alternatively, one or more radial projections 132 may extend circumferentially around the perimeter of the balloon. For example, a radial projection 132 may extend fully around the circumference of the balloon (or around the inflatable portion of the balloon), as an annular ring. As another example, a radial projection 132 may extend partially around the circumference of the balloon (or around the inflatable portion of the balloon), as an arcuate projection.

### C. Valve

As described above, the pump body 120 may further include at least one inlet valve 140 configured to receive fluid along the flow axis of the conduit. In this manner, flow entering the pump body 120 may travel in an axial flow direction, with little to no radial flow component orthogonal to the flow axis. The inlet valve 140 may be a one-way valve with a preferential flow direction between an upstream side and a downstream side, where the one-way valve permits flow into the pump body 120 through the inflow region 120a, while substantially preventing flow out of the pump body 120 through the inflow region 120a. Accordingly, the inlet valve 140 may be configured to operate between an open state in which flow in a first direction (e.g., into the pump body 120, from the upstream side to the downstream side) is permitted, and a closed state in which flow in a second direction opposite the first direction (e.g., out of the pump body 120, from the downstream side to the upstream side) is substantially prevented. For example, in the open state of the inlet valve 140, the valve leaflets may be arranged such that the leaflets do not coapt, while in the closed state of the inlet valve 140, the valve leaflets may be arranged such that the leaflets coapt.

The inlet valve 140 may be configured to repeatedly transition between the closed state and the open state over multiple valve cycles, where a single valve cycle includes various valve phases including (i) transition from the closed state to the open state, (ii) open state, (iii) transition from the open state to the closed state, and (iv) closed state. This cyclical operation of the inlet valve 140 may, for example, passively occur as the result of fluid momentum in pump device (e.g., similar to that described herein with respect to FIGS. 2A-2E). In some variations, the inlet valve 140 may be configured to have a fast response time for transitioning from the closed state to the open state, and from the open state to the closed state, and may have such a fast response time at high frequency over a sustained period of time or number of valve cycles (e.g., high fatigue resistance.).

In some variations, the inlet valve 140 may be configured to transition between the closed and open states in a transition time of between about 12 ms and about 200 ms. In some variations, the inlet valve 140 may be a passive valve configured to open and close in response to pressure change, though in some variations the inlet valve 140 may additionally or alternatively be an active valve whose opening and closure may be controlled by a suitable actuator.

In some variations, the inlet valve 140 may be configured to have a fast response time for transitioning between the open and closed states, particularly at a high frequency over a sustained period of time (e.g., high fatigue resistance). For example, in some variations, the inlet valve 140 may be configured to transition from the closed state to the open state (e.g., at least about 90% of the valve orifice area open) within about 10 milliseconds or less, about 5 milliseconds or less, about 4 milliseconds or less, about 3 milliseconds or less, about 2 milliseconds or less, about 1.5 milliseconds or less, or about 1 millisecond or less. Additionally or alternatively, the inlet valve 140 may be configured to transition from the open state (e.g., at least about 90% of the valve orifice area open) to the closed state within about 20 milliseconds or less, about 15 milliseconds or less, about 10 milliseconds or less, about 7 milliseconds or less, about 4 milliseconds or less, about 3 milliseconds or less, about 2 milliseconds or less, about 1.5 milliseconds or less, or about 1 millisecond or less.

For example, in some variations, the inlet valve 140 may be operated to transition from the closed state to the open state over a duration of no more than about 20% of the valve cycle (e.g., no more than about 15% of the valve cycle). Additionally, or alternatively, the inlet valve 140 may be operated to transition from the open state to the closed state over a duration of no more than about 20% of the valve cycle. Furthermore, the inlet valve 140 may additionally or alternatively be operated to be in the open state for at least about 50% of the valve cycle, and/or operated to be in the closed state for no more than about 15% of the valve cycle. However, in some variations, the amount of time spent in various phases of valve cycle may vary at least in part on the operating frequency of the volume displacement member. Table 1 illustrates example durations of each valve cycle expressed in time, for an example circulatory assist device operated at various pump frequencies. Table 2 illustrates example durations of each valve phase expressed in percent of valve cycle, for an example circulatory assist device operated at various pump frequencies. While Tables 1 and 2 illustrate example durations of valve phases for selected pump frequencies, it should be understood that these values represent envelopes of suitable metrics that may vary between the pump frequencies of 600 bpm and 1800 bpm.

TABLE 1
Example valve phase durations (time)

| | | Closed | | Open to | Total Valve |
|---|---|---|---|---|---|
| Frequency | Closed | to Open | Open | Closed | Cycle Time |
| (bpm) | (ms) | (ms) | (ms) | (ms) | (ms) |
| 1800 | 4.5 | 4.9 | 17.5 | 6.6 | 33.5 |
| 1200 | 7.3 | 7.0 | 19.5 | 16.3 | 50.0 |
| 600 | 50.7 | 6.7 | 36.6 | 6.0 | 100.00 |

TABLE 2
Example valve phase durations (% valve cycle)

| Frequency | | Closed | | Open to |
|---|---|---|---|---|
| (bpm) | Closed | to Open | Open | Closed |
| 1800 | 13% | 15% | 52% | 20% |
| 1200 | 15% | 14% | 39% | 33% |
| 600 | 51% | 7% | 37% | 6% |

Furthermore, in some variations the inlet valve 140 may be configured to withstand high pressure applied in the direction opposite of the preferential flow direction, such as those that the inlet valve 140 may experience under the high frequency pumping action of the pump body 120. For example, in some variations the inlet valve 140 may be configured to maintain its closed state (e.g., with little to no backflow, or little to no fluid leakage from the downstream side to the upstream side) for at least a brief period of time (e.g., at least about 2 milliseconds, at least about 3 milliseconds, at least about 5 milliseconds, at least about 10 milliseconds, or at least about 15 milliseconds, etc.), even under high pressure differentials across the inlet valve. For example, the inlet valve 140 may be configured to maintain the closed state without allowing blood flow from the downstream side to the upstream side under a pressure differential of up to at least between about 500 mmHg and about 1000 mmHg from the downstream side to the upstream side, where positive force is applied in the direction opposite of the preferential flow direction. For example, the inlet valve 140 may be configured to maintain its closed state under an applied pressure differential from the downstream side to the upstream side of up to at least about 500 mmHG, or up to at least about 700 mmHg, or up to at least about 900 mmHg.

In some variations, the inlet valve 140 may be configured to transition to (and/or maintain) the open state in response to a low pressure applied in the preferential flow direction, which may help to maintain momentum of flow through the pump body and/or help maintain a fast opening response time (e.g., low valve cracking pressure). For example, in some variations, the inlet valve 140 may be configured to transition to and/or maintain its open state under a pressure differential that is no more than between about 40 mmHg and about 60 mmHg, where positive force is applied in the direction of the preferential flow direction. For example, the inlet valve 140 may be configured to transition to and/or maintain its open state under an applied pressure differential of less than about 60 mmHg, less than about 50 mmHg, less than about 40 mmHg, less than 30 mmHg, or less than about 20 mm Hg.

In some variations, the inlet valve 140 is configured to maintain a certain minimum amount of geometric orifice area over at least a threshold amount of time while in use, to facilitate the conveyance of at least an expected amount of volumetric flow into the pump body 120, thereby facilitating a certain pump throughput. For example, the inlet valve 140 may be configured to have a geometric orifice area of at least about 40 mm² (e.g., at least about 45 mm²) at 25 Hz (or 1500 beats per minute).

Additionally, in some variations the inlet valve 140 is configured to be sufficiently durable to maintain a sufficient level of performance while in use. For example, in variations in which the inlet valve 140 includes a plurality of leaflets, the inlet valve 140 may be configured to have reduced wear due to leaflet contact, reduced risk of tearing due to high strains as leaflets move, and/or reduced creep, etc. In some variations, for example, the inlet valve 140 may be configured to operate with any of the above-described valve cycles (including response times for transitioning between closed and open states), at any of the above-described pressure differentials across the inlet valve 140, while exhibiting any of the above-described geometric orifice area performance criteria, over at least 50 million valve cycles.

Many of the above-described characteristics of the inlet valve 140 may be achieved with one or more valve features described in further detail below, implemented alone and/or in combination in an inlet valve 140 design. Although the inlet valve 140 is primarily described herein as a tricuspid valve, it should be understood that various details similarly apply to other variations of multi-leaflet valves (e.g., bicuspid valves).

In some variations, the inlet valve 140 is a multi-leaflet valve including a plurality of leaflets. For example, the inlet valve 140 may be a tricuspid valve, or a bicuspid valve. FIGS. 4A and 4B are schematic illustrations of an example circulatory assist device 400 including an inlet valve 140 in the form of a tricuspid valve. The circulatory assist device 400 may be generally similar to circulatory assist device 100. For example, like circulatory assist device 100, the circulatory assist device 400 may include a pump body 120 with a conduit including an inflow region 120a, an outflow region 120c, and an intermediate region 120b between the inflow region 120a and the outflow region 120a. The pump body 120 may include a support 122 (e.g., nitinol frame) and at least one membrane 124 adjacent to the support 122. As shown in FIG. 4A, the support 122 may be closed at its distal inflow end (e.g., with a connector 126) and open at its proximal outflow end. The circulatory assist device 400 may further include a volume displacement member 130 arranged in the conduit, and a catheter 110 configured to provide functional support to pump body 120 and/or volume displacement member 130 (e.g., conveying an inflation fluid to inflate and/or deflate a balloon-type volume displacement member 130).

The inlet valve 140 may include a plurality of leaflets configured to passively move in response to a pressure differential. For example, the inlet valve 140 may be a tricuspid valve with three leaflets. The inlet valve 140 may be configured to operate in an open state in which the leaflets are not coapting against another, and in a closed state in which the leaflets are coapting against one another (and/or against a guidewire and/or guidewire housing member extending through the inlet valve 140, as described in further detail herein). As shown in FIGS. 4A and 4B, the inlet valve 140 may be oriented such that its preferential flow direction (in which the inlet valve 140 opens and permits flow) is in a distal-to-proximal direction. Accordingly, the inlet valve 140 may be configured to open in response to a negative pressure in the pump body 120 (whereby fluid is drawn from outside the pump body 120 into the distal inflow region 120a).

In some variations, the inlet valve 140 may be formed from a continuous piece of material (e.g., polymer). For example, FIG. 5 depicts an example variation of a tricuspid valve 540 that is formed from a single piece of material (e.g., polymer) with multiple leaflets 142a, 142b, and 142c and a valve base 146 functioning as a structure to which the leaflets are attached. The material may, for example, be thermoformed into a preset shape defining the leaflets 142a, 142b, and 142c as an integrally formed structure (in contrast to separately forming the leaflets and subsequently joining or assembling the leaflets when manufacturing the inlet valve 140, for example). In some variations, at least a portion of the inlet valve may be joined to (e.g., coupled to (such as via chemical weld), integrally formed with) one or more membranes 124 of the conduit, such as an inner membrane 124a and/or outer membrane 124b such as that described herein with respect to FIGS. 3A-3C.

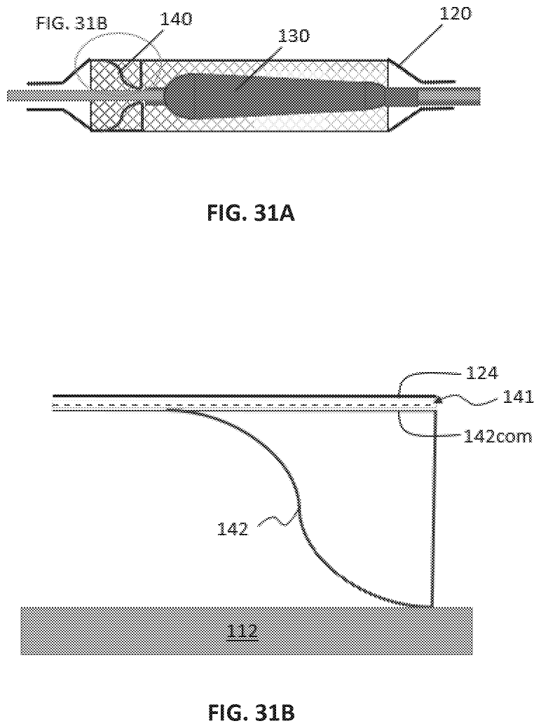

For example, as shown in FIGS. 31A and 31B, in some variations the inlet valve 140 may be joined to a membrane 124 (e.g., inner membrane) of the conduit, such as at the commissures and/or curvature, and/or other periphery of the inlet valve 124. The side cross-sectional view of FIG. 31B schematically illustrates, or example, a commissure region 142com of the inlet valve that is joined to a membrane 124 of the conduit, while the free edge of leaflet 142 remains unattached to the membrane 124. In some variations, the inlet valve 140 may be formed separately (e.g., via injection molding, spray coating, and/or dip coating) of a first polymer, and a solution of a second polymer for the membrane may be administered in a spray coating and/or dip coating process over selected portions of the inlet valve (e.g., with masking of the leaflets). As such, an intermixed region 141 including a combination of the first and second polymers may be formed between the commissure region 142com and the membrane 124. The first and second polymers may be different materials, or may be the same material. In some variations, the juncture between the inlet valve and the membrane 124 may be reinforced by additional targeted application of the second polymer along an attachment edge (e.g., tip of commissure and/or curvature of valve) such as by syringe or brush, and/or a second coating of the second polymer may be administered via spray coating and/or dip coating to complete the formation of the membrane 124 with the inlet valve 124 joined thereto. In variations in which the membrane 124 is an inner membrane of the conduit, the valve-membrane assembly may then be subjected to encapsulation and/or formation of the rest of the conduit (e.g., support, outer membrane, etc.) around the valve-membrane assembly.

However, in some variations, some or all of the leaflets and/or the valve body in the inlet valve may be separately formed and subsequently joined to form the inlet valve 140 (e.g., with a connector such as a ring). In some variations, the inlet valve 140 may be formed from multiple different materials (e.g., different polymers). Furthermore, in some variations, the single or multiple pieces of material may be coated with a suitable material (e.g., reinforcement material) such as through dip coating, spray coating, etc.

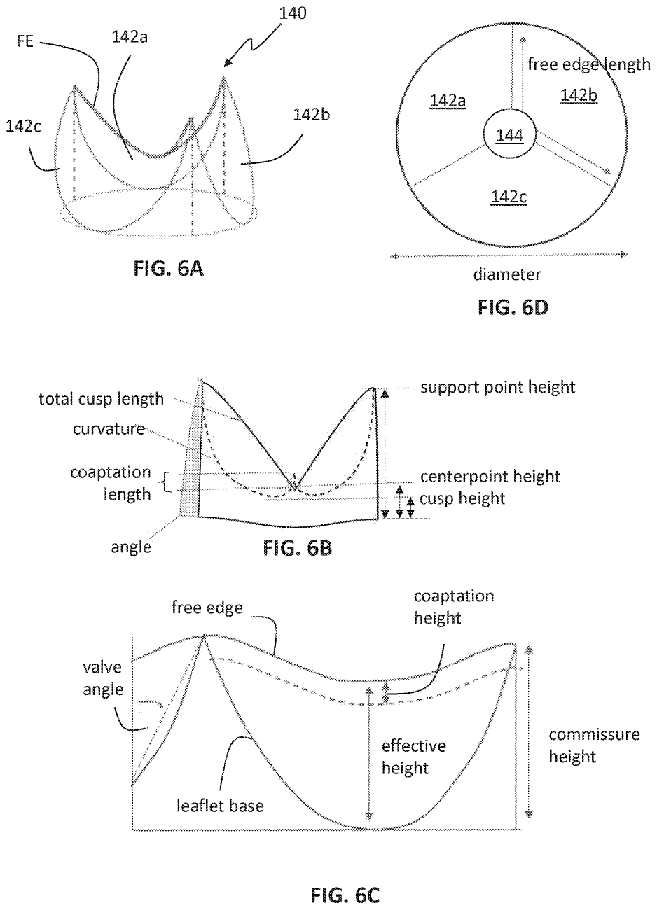

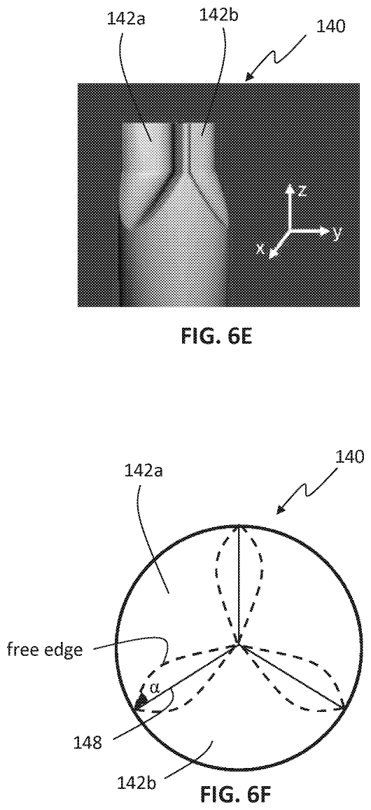

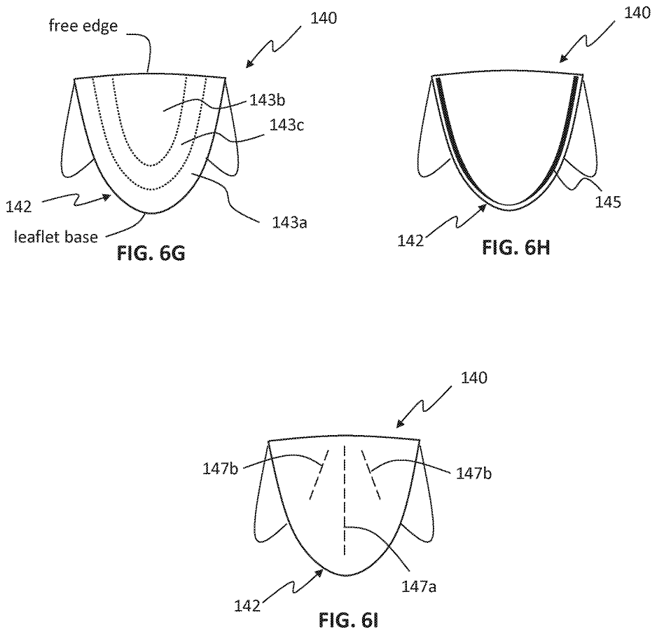

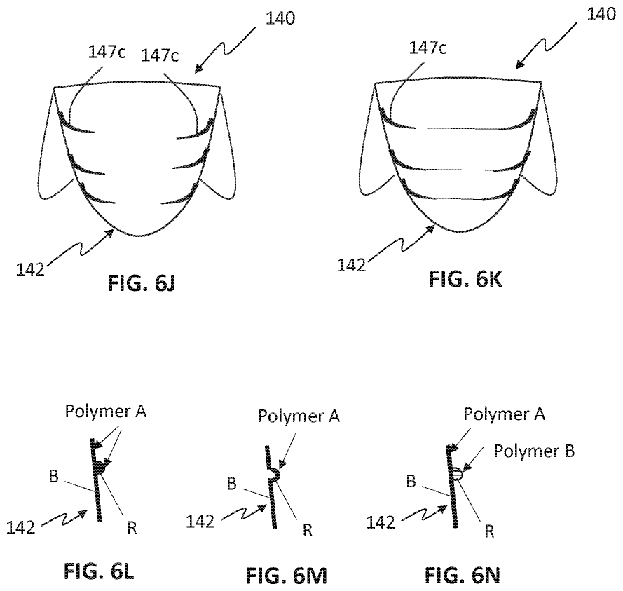

FIGS. 6A-6D illustrate various aspects of an example inlet valve 140 including three leaflets. FIG. 6A is a schematic illustration of the contours of the leaflets of a tricuspid inlet valve 140, including three leaflets 142a, 142b, and 142c coapting together in the closed state of the inlet valve 140. When the inlet valve 140 is in a closed state as shown in FIG. 6A, inlet valve 140 may have a contoured valve profile FE where the free edges of adjacent leaflets are in contact with one another. FIGS. 6B-6D are schematic illustrations of the tricuspid inlet valve 140, with various geometric features labeled. Many of these geometric features are referenced below.

The inlet valve 140 may be arranged generally in the inflow region 120a of the pump body 120. In some variations, the inlet valve 140 may be at least partially longitudinally overlapping a distal portion of the membrane 124 of the pump body 120, such that fluid entering the pump body 120 through the inlet valve 140 is contained in a fluid-impermeable portion of the pump body 120. In some variations, the inlet valve 140 may be coupled directly or indirectly to the support 122. For example, a periphery of the inlet valve 140 (e.g., along at least a portion of the support point height) may be coupled directly to a distal portion of the support 122.

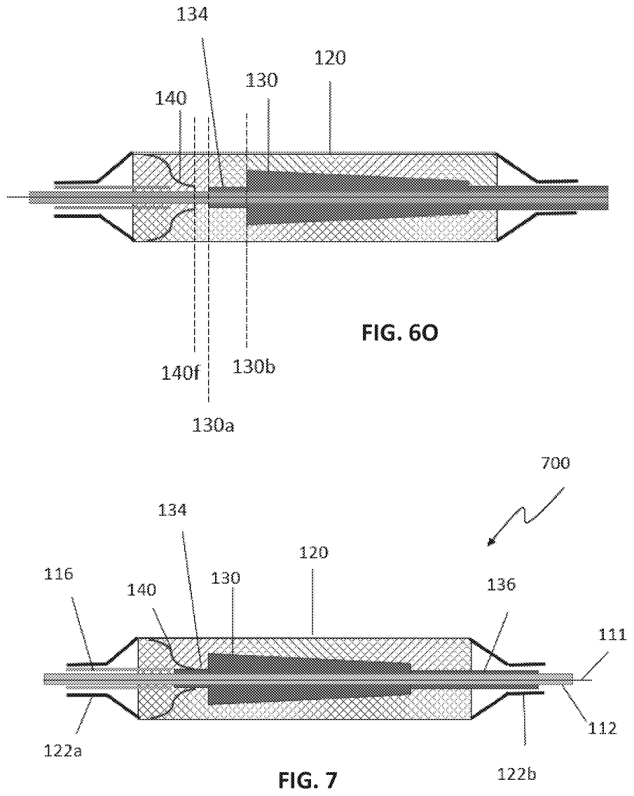

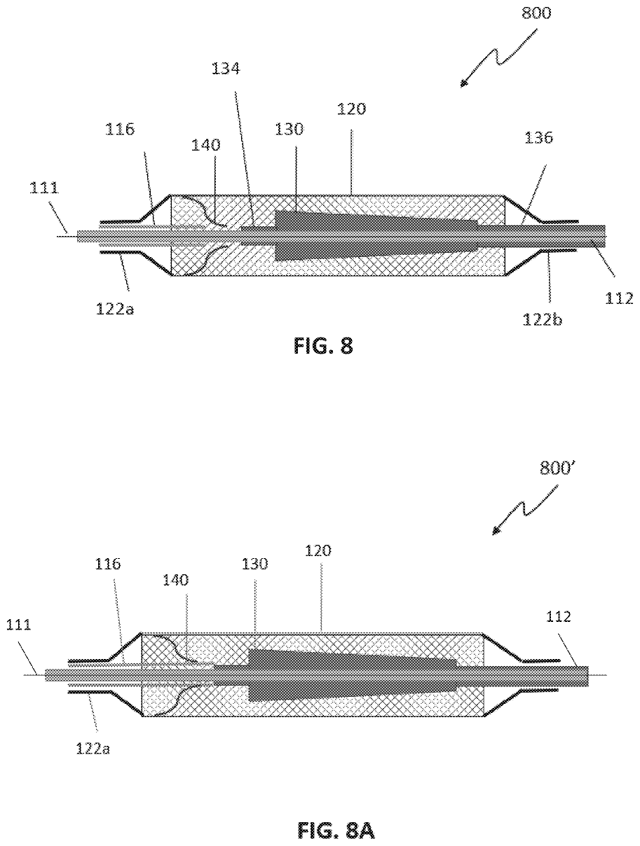

In some variations, the inlet valve 140 in its closed state may be configured to seal or close around one or more various structures that are in and/or pass through the pump body 120. Such structures passing through the inlet valve may, in some variations, help reduce or substantially inhibit prolapsing of the valve leaflets. For example, the longitudinal position of the inlet valve 140, the support point height, and/or other geometric features of the inlet valve 140 may be configured such that the leaflets of the inlet valve 140 seal around one or more suitable structures. In some variations, such as that shown in FIG. 7, the leaflets of the inlet valve 140 may be configured to seal or otherwise close around a distal neck portion of the volume displacement member 130 (e.g., balloon), and/or other portion of the volume displacement member 130 (e.g., at least a portion of an expandable region of the volume displacement member 130). In some variations, such as that shown in FIG. 8, the leaflets of the inlet valve 140 may be configured to seal or otherwise close around a guidewire housing member 112 and/or a guidewire passing through the guidewire housing member 112.

In some variations, the inlet valve 140 may define an aperture between its coapting leaflets when the inlet valve 140 is in the closed state. The aperture may function to enable one or more structures to longitudinally pass through the valve plane of the inlet valve 140. As described in further detail herein, for example, a guidewire and/or a guidewire housing member may pass through the aperture of the inlet valve 140 (both when the inlet valve 140 is in the open state and in the closed state), such that the guidewire and/or guidewire housing member may extend from a proximal side of the inlet valve 140 to a distal side of the inlet valve 140. In some variations, the aperture may be substantially complementary to the outer profile of the structure(s) passing through the inlet valve to enable the inlet valve 140 to seal in a substantially fluid-tight manner around such structure(s) when the inlet valve 140 is in a closed state. For example, the aperture may be generally circular to enable sealing around the guidewire, guidewire housing member, and/or other structure passing through the inlet valve 140, and/or increase contact surface area between any of these components at the inlet valve 140 such that the leaflets may help provide support to the leaflets.

For example, as shown in FIG. 6D, one or more of the leaflets (e.g., 142a, 142b, 142c) may include a cutout at its free edge such that a negative space forming aperture 144 remains when the leaflets articulate against each other in the closed state. In some variations, each leaflet may include an arcuate cutout. For example, each leaflet may include an arcuate cutout in the shape of a circular segment, such that collectively, the arcuate cutouts of the multiple leaflets define a generally circular aperture 144 (e.g., as shown in FIG. 6D each of the three leaflets 142a, 142b, and 142c may include an arcuate cutout of approximately 120 degrees). In some variations, the diameter of a circular aperture 144 may be between about 1 mm and about 2 mm, or between about 1 mm and about 3 mm. As another example, each leaflet may include an arcuate cutout in the shape of one side of a curvilinear polygon, such that collectively the arcuate cutouts of the multiple leaflets define an aperture 144 in the shape of the curvilinear polygon (e.g., as shown in FIG. 10B, each of the three leaflets 142a, 142b, and 142c may include a cutout in the shape of a deltoid curve, such that collectively the cutouts of the three leaflets form a curvilinear triangle when the inlet valve 140 is in the closed state). However, the cutouts may have any suitable shape to form an aperture 144 of any suitable profile. In some variations, the perimeter of the leaflet free edges around the aperture 144 may include textural features (e.g., surface roughness) to help increase friction with the guidewire or guidewire housing member, thereby further improving support to the leaflets during operation, especially high frequency operation.

As described below, the individual leaflets of the inlet valve 140 may include various further features that contribute to the inlet valve having the desired characteristics described above (e.g., fast opening and closing response time, ability to withstand high pressures, ability to open in response to a low pressure gradient, ability to maintain good orifice area both short-term and long-term, durability, etc.).

In some variations, the leaflets of the inlet valve 140 may include excess material (e.g., “wavy” leaflets). Such excess material may result in the cuspids being larger than necessary to ensure coaptation, which helps to reduce tension or stretching in the leaflets during valve operation. Accordingly, the excess material allows the leaflets to absorb more pressure and avoid extreme movements, thereby reducing strain and wear on the leaflets and/or at the commissures of the valve, as well as improving durability. In some variations, the amount of excess material in a leaflet is such that when the inlet valve 140 is in the closed state, the leaflet is not stretched taught. For example, the cross-sectional profile of each leaflet can be characterized as having a series of local maxima and/or minima, or the free edge of the leaflet has the shape of a pseudo-periodic curve. In some variations, the amount of excess material at the free edge of a leaflet may be such that when the inlet valve 140 is in the closed state (e.g., during coaptation), the length of material on the leaflet contacting a coapting leaflet is between about 5% and about 10%, between about 5% and about 15%, between about 5% and about 20%, between about 5% and about 30%, or about 5%, or about 10%, or about 15%, or about 20%, or about 25%, or about 30% more than the length of material on the leaflet not contacting the coapting leaflet is about 1:1, or about 1.5:1, or about 2:1.

In some variations, the inlet valve 140 may have a valve orifice area configured to facilitate sufficient blood flow through the circulatory assist device, while maintaining suitably fast valve opening and valve closing response times. For example, in some variations, the valve orifice area of the inlet valve 140 may be between about 30% and about 80% of the average cross-sectional area of the conduit, when the valve is in the open state while operated at high frequencies (e.g., at least 500 bpm, at least 1000 bpm). Additionally or alternatively, in some variations, the valve orifice area of the inlet valve 140 may be between about 10% and about 55% of the maximum cross-sectional area of the conduit at the location of the valve (e.g., base of the valve joined to the conduit membrane), when the valve is in the open state while operated at high frequences (e.g., at least 500 bpm, at least 1000 bpm). Furthermore, in some variations, the valve orifice area of the inlet valve 140 may be between about 15% and about 98%, or between about 15% and about 90%, of the maximum cross-sectional area of the conduit at the location of the valve (e.g., base of the valve joined to the conduit membrane).

In some variations, material and/or thickness of at least some of the leaflets may be selected to be sufficiently thin and stiff to promote fast opening in response to low pressure gradients across the valve, by reducing the amount of momentum required to open the valve. Furthermore, at least some of the leaflets may be sufficiently thin and stiff to allow for fast closing response times. For example, in some variations, the leaflets may include a material having a hardness of at least about 80 A (e.g., 80 A or higher, 85 A or higher) and a wall thickness of between about 10 μm and about 250 μm, or between about 50 μm and about 250 μm. For example, suitable leaflet materials may include any one or more of a polyether, polycarbonate, silicone polycarbonate, or aromatic thermoplastic polyurethane material. In some variations, the leaflets may include a material that shows a limited change in dynamic modulus over a wide frequency range; for example, a material that has a dynamic modulus that varies less than 20% between lower frequencies (e.g., 400 bpm) and higher frequencies (e.g., 2500 bpm). Additionally or alternatively, the leaflets may include a material that exhibits a minimum dynamic modulus phase shift (e.g., close to zero) between stress and strain, which is advantageous for durability during high frequency movement of the leaflet. In some variations, the leaflets may include a material that exhibits a dynamic modulus that does not change significantly at any given operational frequency over an extended number of cycles; for example, in some variations such a material may exhibit a dynamic modulus that does not change more than about 20% over the course of around 50 million cycles at any given operational frequency.

In some variations, at least some of the leaflets may include multiple layers of different materials to form a composite leaflet structure. Such a composite leaflet structure, may, for example, include one or more layers of a softer, elastic material (which may, for example, help contribute to faster valve response times) and one or more layers of a harder, stiffer material (which may, for example, reduce stress on the valve and/or improve durability of the valve), thereby resulting in a valve that benefits from advantages of both soft and hard materials. A leaflet may, in some variations, include two, three, or more layers of materials. Hard and soft materials may be interleaved or alternated, such as described in the examples below. Furthermore, in some variations, the composite leaflet structure may be coated (e.g., with a spray coating) to help secure the bond between layers and deter delamination of the composite leaflet structure.

For example, a dual layer leaflet may include a first layer of a harder polymer (e.g., durometer of at least 55 D) and a second layer of a softer polymer (e.g., durometer of less than about 55 D). In this example, the harder polymer may be on an inner layer of the leaflet or outer layer of the leaflet relative to the valve opening. In some variations, the thicknesses of the first and second layers may be the same (e.g., about 50% of the leaflet thickness is the first layer, and about 50% of the leaflet thickness is the second layer). However, in some variations, the thicknesses of the first and second layers may be different. For example, in some variations, the first layer with the harder polymer may form between about 5% and about 50% of the overall leaflet thickness and the second layer with the softer polymer may form between about 95% and about 50% of the overall leaflet thickness. Alternatively, the second layer with the softer polymer may form between about 5% and about 50% of the overall leaflet thickness and the first layer with the harder polymer may form between about 95% and about 50% of the overall leaflet thickness.

As another example, a three-layer leaflet may include inner and outer layers formed of a harder polymer or different harder polymers (e.g., durometer of at least 55 D) and a middle layer sandwiched between the inner and outer layers formed of a softer polymer (e.g., durometer of less than about 55 D). Alternatively, a three-layer leaflet may include inner and outer layers formed of a softer polymer or different softer polymers, and a middle layer sandwiched between the inner and outer layers formed of a harder polymer. In some variations, the thicknesses of the three layers may be about equal, or may vary. For example, in some variations, the inner and outer layers may each form between about 5% and about 48% of the overall leaflet thickness, and the middle layer may form between about 90% and about 4% of the overall leaflet thickness.

Furthermore, in some variations, at least some of the leaflets may include a different material at a coaptation surface compared to the rest of the leaflet body. For example, a leaflet body may include a first material, while the surface area along the coaptation height of one or more leaflets may include a second material different from the first material, where the second material is softer than the first material. In some variations, the softer material at the coaptation surface may help to reduce wear of the coaptation interface over time, thereby improving durability of the inlet valve 140. For example, in some variations the second material along the coaptation surface may include a material having a hardness of between about 70 A and about 80 A, while the first material in the leaflet body may include a material having a hardness of 80 A or higher (e.g., at least about 80 A to about 72 D). For example, in some variations the second material along the coaptation surface may include a material having a hardness of between about 70 A) and about 90 A, while the first material in the leaflet body may include a material having a hardness of about 90 A or higher (e.g., at least about 90 A to about 72 D). Additionally or alternatively, in some variations, at least some of the leaflets may include a focal reinforcement structure at a coaptation surface, such as one or more ribs (e.g., embedded or overmolded member within the leaflet, or thicker region of leaflet material), that increases the durability of the coaptation surface and the overall durability of the inlet valve. For example, as shown in FIG. 6H, in some variations, a leaflet 142 may, for example, include a reinforcement member 145 (e.g., rib) located along a free edge region of the leaflet that tapers down in thickness toward a central or midline region of the free edge of the leaflet 142. However, in other examples, a leaflet 142 may include a reinforcement member 145 that does not taper in thickness, or multiple reinforcement members 145 (e.g., two lateral reinforcement members extending along the free edge of the leaflet and opposing each other across a midline of the leaflet). Furthermore, in some variations, the reinforcement members described below with respect to FIGS. 6I, 6J, and 6K (e.g., reinforcement members 147a, 147b, 147c) may additionally or alternatively function as focal reinforcement members, such as to help increase durability of the coaptation surface and/or overall durability of the inlet valve.

In some variations, the inlet valve 140 may include at least one leaflet (e.g., some or all leaflets) having variable bending stiffness along its leaflet body. For example, at least one leaflet of the inlet valve 140 may include a first region having a first bending stiffness and a second region having a second bending stiffness, where the second bending stiffness is greater than the first bending stiffness. In some variations, more than two regions of the leaflet can have different bending stiffnesses. For example, as shown in FIG. 6G, a schematic of an example leaflet 142 in an inlet valve 140 can include a first leaflet region 143a having a first bending stiffness and a second leaflet region 143b having a second bending stiffness different from the first bending stiffness. Optionally, the leaflet can further include a third leaflet region 143c having a third bending stiffness that is different from the first and second bending stiffnesses. The decrease or increase in bending stiffness of the leaflet across different regions of the leaflet may be linear or stepwise (e.g., in bands). As such, although FIG. 6G identifies three regions 143a, 143b, and 143c that may have different bending stiffnesses, in other examples a leaflet 142 may include two, three, four, five, six, or more regions that vary linearly or in discretely in bending stiffness.

In some variations, bending stiffness (e.g., bending stiffness inherent to the leaflet structure, aside from the attachment of the leaflet to the rest of the valve structure) may vary from an interior region of the leaflet to the outer edge of the leaflet. For example, bending stiffness may be lower in leaflet regions proximate to the free edge of the leaflet, compared to leaflet regions proximate to the base of the leaflet. For example, with reference to FIG. 6G, a first leaflet region 143a located in a base region of the leaflet may have a first bending stiffness, and a second leaflet region 143b located along or proximate to the free edge of the leaflet may have a second bending stiffness, where the first leaflet region 143a may have a greater bending stiffness than the second leaflet region 143b (or the second leaflet region 143b may have a lower bending stiffness than the first leaflet region 143a). A third leaflet region 143c located in an arc between the first and second leaflet regions may have a third bending stiffness that is between the first and second bending stiffnesses. It has been discovered that generally, lower bending stiffness in the interior or free edge regions of the leaflet may be advantageous for allowing fast dynamic movement of the leaflet (e.g., for faster response or transition times between the closed and open valve states), while the higher bending stiffness along the base region of the leaflet may be advantageous for reducing stress on the valve that may occur during high frequency operation of the valve. As such, this variance in bending stiffness may be particularly beneficial for the inlet valve 140 to be durable and operate with fast elastic response times at high frequencies (e.g., between about 300 bpm and 5000 bpm). This variance in bending stiffness may be disadvantageous for valves configured for operation at lower frequencies (e.g., artificial aortic or other heart valves) where higher bending stiffnesses at the outer free edges of the leaflets may undesirably result in reduced valve orifice area and reduced blood flow. However, in the application of high frequency operation, the variance in bending stiffness may surprisingly be advantageous for at least the above-described reasons.

In some variations, the variance in bending stiffness along a leaflet may be accomplished at least in part by providing a variance in leaflet thickness. In addition to contributing to varying bending stiffness as described above, varying leaflet thickness including thinner interior leaflet regions can furthermore advantageously contribute to a lower crimping profile for the overall circulatory assist device. For example, the thickness of the leaflet may taper linearly or discretely from a greater thickness at the commissure support point toward a smaller thickness at a midline or center of the leaflet, and/or from a greater thickness at a base of the leaflet toward a smaller thickness at a free edge of the leaflet. In some variations, for example, a second leaflet region at an interior or free edge region of the leaflet (e.g., leaflet region 143b) may have a thickness that is between about 45% and about 55% of the thickness of a first leaflet region at a base region (e.g., leaflet region 143a). Furthermore, in some variations, a third leaflet region (e.g., leaflet region 143c) between the first and second leaflet regions may have a thickness that is between about 70% and 80% of the thickness of the first leaflet region at the base region of the leaflet. In one illustrative example, with reference to FIG. 6G, a leaflet with varying thickness may include a first leaflet region 143a near a base of the leaflet with a thickness of about 200 μm, a second leaflet region 143b near a free edge of the leaflet with a thickness of about 90 μm, and a third leaflet region 143c between the first and second leaflet regions with a thickness of about 130 μm. In another illustrative example, a leaflet with varying thickness may include a first leaflet region 143a near a base of the leaflet with a thickness of about 120 μm, a second leaflet region 143b near a free edge of the leaflet with a thickness of about 60 μm, and a third leaflet region 143c between the first and second leaflet regions with a thickness of about 90 μm.

A leaflet with varying thickness may be formed in various suitable manners. For example, a leaflet may be formed at least in part through dip coating, where the valve is held with the leaflets oriented downwards as the coating solution collects and drips off the free edges of the leaflets, thereby forming thickening at the free edge regions of the leaflets. As another example, a leaflet may be formed through injection molding using a mold with a tapered volume corresponding to the desired thickness taper.

Additionally or alternatively, the variance in bending stiffness along a leaflet may be accomplished at least in part by providing different material(s) in different regions of the leaflet body. For example, in some variations, the durometer of the leaflet material(s) may scale from a higher durometer at the commissure support point toward a lower durometer at a midline or center of the leaflet, and/or from a higher durometer at a base of the leaflet toward a lower durometer at a free edge of the leaflet. In some variations, for example, a second leaflet region at an interior or free edge region of the leaflet (e.g., leaflet region 143b) may have a durometer that is between about 35% and about 65% (e.g., about 50%) of the durometer of a first leaflet region at a base region (e.g., leaflet region 143a). Furthermore, in some variations, a third leaflet region (e.g., leaflet region 143c) between the first and second leaflet regions may have a durometer that is between about 65% and about 85% (e.g., about 75%) of the durometer of the first leaflet region at the base region of the leaflet. As an illustrative example, with reference to FIG. 6G, a leaflet with varying durometer may include a first leaflet region 143a near a base of the leaflet with a durometer of between about 35 D and about 72 D (e.g., above 55 D), and a second leaflet region 143b near a free edge of the leaflet with a durometer of between about 85 A and about 40 D (e.g., below 55 D). The leaflet may, in some examples, further include a third leaflet region 143c between the first and second leaflet regions with a durometer between the durometers of the first and second leaflet regions (e.g., between about 95 A and about 63 D, such as about 55 D).

A leaflet with varying durometer may be formed in various suitable manners. For example, a leaflet may be formed through an injection molding process to form bands of different materials having different durometers. As another example, different materials with different durometers may be layered as a composite to form a different effective durometer in different regions of the leaflet.

In some variations, some or all of the leaflet body may be preshaped (e.g., molded) to help bias the inlet valve toward the closed state and/or reduce stress on the leaflet. For example, the curvature of the cuspid may be molded or otherwise preshaped to preset the valve in a closed state, to help improve elastic recovery of the leaflet body, thereby improving fast closing response times. Additionally or alternatively, the leaflet body may include a reinforcement coating (e.g., dip coating, spray coating) to help preset the valve in a closed state.

Additionally or alternatively, in some variations, some or all of the leaflets may include one or more reinforcement members that may help improve elastic recovery, thereby improving fast closing response time. For example, one or more leaflets may include at least one structural rib, strut, and/or other member etc. arranged along the leaflet body so as to help with elastic recovery and/or bias the leaflet toward the closed state. Such reinforcement members can, for example, be preformed and have a default shape that generally corresponds to a desired curvature of a cusp when the valve is in the closed state (or more contoured than such cusp curvature, so as to “overcorrect” and help further ensure fast closing response time). FIG. 6I is a schematic illustration of example reinforcement members in a leaflet. For example, a leaflet 142 may include at least one reinforcement member 147a arranged along at least a portion of a midline of the leaflet 142 extending generally from the base to the free edge of the leaflet, and/or at least one reinforcement member 147b extending laterally from the midline (e.g., branching away from the midline). FIG. 6J is a schematic illustration of more example reinforcement members in a leaflet 142, including at least one reinforcement member 147c extending laterally from an edge of the leaflet toward the midline of the leaflet 142. The example shown in FIG. 6J includes multiple sets of such laterally extending reinforcement members 147c that are mirrored across the midline (e.g., with a gap at the midline that separates laterally opposing reinforcement members 147c). However, in some variations such as that shown in FIG. 6K, a leaflet may include one or more continuous reinforcement members 147c that extend laterally across the midline, and/or across the entire leaflet body (e.g., no gap at the midline that separates laterally opposing reinforcement members 147c). In some variations, the reinforcement member(s) 145 described above with reference to FIG. 6H may additionally or alternatively function to assist with elastic recovery. Similar to that described above, in some variations, one or more reinforcement members may taper down in thickness toward a central or midline region (e.g., as shown in FIG. 6J and FIG. 6K).

In variations having multiple reinforcement members, such reinforcement members may be joined or separate, and/or may be arranged in a bilaterally symmetric manner to help improve elastic recovery of the leaflet in a balanced manner. The reinforcement member may be integrally formed with the leaflet body (e.g., molded), or may be formed separately from the leaflet body and subsequently joined to the leaflet body. As another example of reinforcement features, one or more leaflets may include corrugations and/or thickenings (e.g., selective portions of the leaflet body with thicker leaflet wall) to reinforce the leaflet body toward the closed state.

FIGS. 6L-6N illustrate cross-sectional views of the structure of various examples of reinforcement members R (e.g., reinforcement members 145, 147a, 147b, 147c, etc.) in a leaflet 142. As shown in FIG. 6L, in some variations, a reinforcement member R may have a substantially filled or solid cross-sectional volume and formed of the same material (e.g., “Polymer A”) as the leaflet body B. For example, the reinforcement member R may be integrally formed with the leaflet body B through injection molding of the leaflet body B, or formed separately from and later joined to the leaflet body B. In some variations, as shown in FIG. 6M, a reinforcement member R may include a deformed portion of the leaflet body B and formed of the same material (e.g., “Polymer A”) as the leaflet body B, such as through injection or other suitable molding process. Furthermore, as shown in FIG. 6N, in some variations a reinforcement member R may be formed from a different material (e.g., “Polymer B”) than the leaflet body B (e.g., “Polymer A”), and later joined to the leaflet body B. Although the reinforcement members R shown in FIGS. 6L-6N have a generally circular cross-sectional shape or a segment or portion thereof, it should be understood that in other variations the reinforcement members R may have any suitable cross-sectional shape or a segment or portion thereof (e.g., elliptical, semi-circular, rectangular, etc.).

Additionally or alternatively, in some variations, valve angle may be configured to help improve fast opening response time and/or fast closing time. An example inlet valve 140 shown in three dimensions in XYZ space is shown in FIG. 6E. Taking the z-axis as indicating the height of the valve as shown in FIG. 6E, the valve angle (also shown in FIG. 6C) is the angle of the valve in the y-z plane. For example, in some variations, valve angle may be between about 30 degrees and about 90 degrees, which may reduce closing volume (e.g., the volume through which the leaflets sweep while opening and closing), and/or the amount of travel time for the leaflets to transition between the valve's open and closed states, while still maintaining sufficient flow through the inlet valve. In some variations, because a reduction in the closing volume also reduces surface area across which pressure is applied on the leaflet, a valve angle of between about 30 degrees and about 90 degrees may additionally help improve the resistance of the inlet valve 140 to high pressure.

Additionally or alternatively, in some variations, cutoff angle of the inlet valve may be configured to help achieve a better closure of the inlet valve and/or reduce stress placed on the commissure of the inlet valve during cyclical operation. As shown in FIG. 6F and with reference to the coordinates shown in FIG. 6E, the cutoff angle is the angle in the x-y plane formed between the commissure of the inlet valve and the line 148 indicating the geometric division in n equal leaflet parts (shown with n=3 in FIG. 6F for a tri-leaflet valve). For example, in some variations the cutoff angle may be between about zero and about 45 degrees, or between about 5 and about 30 degrees, so as to improve valve closure and/or result in less stress on the commissure during valve operation. Generally, in some variations the smaller the cutoff angle, the better the valve closure and/or less commissure stress.

Additionally or alternatively, in some variations the coaptation length in the inlet valve 140 may be reduced to a sufficient length, so as to increase durability of the inlet valve 140. For example, in some variations the coaptation length may be about 1 mm or less.

Furthermore, in some variations the total cusp length and/or angle may be selected to reduce strain in the inlet valve, thereby increasing durability of the inlet valve 140. For example, in some variations, the angle may be between about 30 degrees and about 60 degrees (e.g., about 45 degrees).

In addition, or alternative to the design of the valve itself, the location of the valve relative to the volume displacement member in the circulatory assist device may help improve flow through the circulatory assist device and/or reduce stress on the valve leaflets. In some variations, the closer the inlet valve is to the volume displacement member along a flow direction of the circulatory assist device, the greater the improvement in flow and the reduction of stress on the leaflets. For example, with reference to an example schematic of a circulatory assist device shown in FIG. 6O, in some variations the distance between the inlet valve 140 and the volume displacement member 130 (e.g., as measured between a free edge 140f of the leaflets on the proximal or outflow side of inlet valve 140, and the nearest end 130b of the expandable or operating volume of the volume displacement member 130) can be between about 1 mm and about 35 mm, or between about 1 mm and about 20 mm. As another example, with reference to FIG. 6O, in some variations the distance between the inlet valve 140 and the volume displacement member 130 (as measured between a free edge 140f of the leaflets on the proximal or outflow side of the inlet valve 140, and the nearest end 130a of a neck of the volume displacement member 130) can be between about 1 mm and about 15 mm.

In some variations, at least a portion of the inlet valve 140 may overlap with a portion of the volume displacement member, which may similarly improve flow and/or reduce stress on the valve leaflets (e.g., by substantially inhibiting prolapse of the leaflets). For example, at least a portion of the volume displacement member (e.g., at least a portion of the distal neck of the volume displacement member denoted between 130a and 130b as shown in FIG. 6O, and/or at least a portion of an expandable portion of the volume displacement member such as a distalmost 1 mm-5 mm region of the expandable portion of the volume displacement member) may be located within the opening of the inlet valve 140, such that the free edge 140f of the leaflets of inlet valve 140 is located axially between points 130a and 130b of the volume displacement member. Without being bound by theory, it is believed that such an overlapping arrangement may improve flow and/or reduce stress on leaflets because expansion of the volume displacement member may help supplement the opening of the inlet valve and/or the leaflets may be supported by contraction of the underlying volume displacement member during closing of the inlet valve. In some variations, for example, the leaflets of the inlet valve 140 and the volume displacement member (e.g., neck) may overlap by between about 1 mm and about 10 mm.

The inlet valve 140 may be configured to transition between a collapsed (e.g., crimped) state with a low profile such as for placement in a delivery catheter, and an expanded operational state with a larger profile such as when the inlet valve 140 is deployed with a circulatory assist device. For example, as described herein, a circulatory assist device may be collapsible to a low profile (e.g., for insertion in a delivery catheter) for delivery to a treatment site, then deployed (e.g., released from the delivery catheter) and expanded to a larger diameter at a treatment site. When the circulatory assist device expands, the inlet valve 140 may correspondingly expand. In some variations, the dimensions of the inlet valve 140 may depend at least in part on the application or intended treatment site for the circulatory assist device. For example, in some variations the inlet valve 140 in its expanded operational state may have an outer diameter of up to about 25 mm, or up to about 15 mm (e.g., between about 10 mm and about 15 mm). In some variations, the inlet valve 140 in its expanded operational state may have an outer dimeter that is between about 1.2 and about 6 times the diameter of the lumen of the delivery catheter. The delivery catheter, in turn, may have a lumen of any suitable diameter such as up to 30F (e.g., for delivery of a circulatory assist device for placement in a right ventricle), or up to about 18F or 6 mm diameter (e.g., for delivery of a circulatory assist device for at least partial placement in an aorta).

### D. Guidewire

In some variations, the circulatory assist system may further include a guidewire to help with positioning of the circulatory assist device in a patient. For example, in operation, the guidewire may be advanced through vasculature of a patient and to a target location (e.g., into the left ventricle) and the pump body 120 may follow the path of the guidewire such that at least a portion of the pump body 120 is at the target location (e.g., at least partially in the left ventricle). For example, as shown in FIGS. 7 and 8, a guidewire 111 may be configured to pass longitudinally through the pump body 120. The guidewire may, for example, include any suitable wire material (e.g., stainless steel, nitinol, etc.). The circulatory assist device may further include a guidewire housing member 112 that passes longitudinally through the pump body 120, where the guidewire housing member 112 includes a lumen that receives the guidewire. In some variations, the guidewire housing member 112 is incorporated in (e.g., received in), or is equivalent to the catheter 110. The guidewire housing member 112 may, for example, function to provide a space through which the guidewire 111 may pass without interfering with other components of the pump body 120. The guidewire housing member 112 may include any suitable flexible material (e.g., polymer material).

In some variations, the guidewire and guidewire housing member may be centered within the pump body 120. For example, the guidewire 111 and guidewire housing member 112 may be configured to extend along a central axis of a volume displacement member 130 that is centered within the pump body 120 and/or through the inlet valve 140. In some variations, radial positioning (e.g., centering) of the guidewire 111 and/or guidewire housing member 112 may be aided at least in part by interaction between the guidewire housing member 112 and the support of the pump body. For example, as shown in FIGS. 7 and 8, in some variations the support may include a necked or narrowed proximal region 122b with a smaller diameter than in the intermediate region 120b of the pump body. The guidewire housing member 112 may pass through the narrowed proximal region 122b with low clearance (e.g., 1 mm) so as to be substantially radially constrained and centered relative to the pump body 120. In some variations, the volume displacement member 130 may similarly have a narrowed proximal neck 136 that passes through the proximal region 122b, such that the volume displacement member 130 is similarly substantially radially constrained and centered relative to the pump body 120.

Additionally or alternatively, radial positioning (e.g., centering) of the guidewire 111 and/or the guidewire housing member 112 may be aided at least in part with a distal support member 116. The support member 116 may include, for example, a tube with a lumen that receives the guidewire 111 and/or the guidewire housing member 112. The support member 116 may include a rigid or semi-rigid material, and may be coupled to a necked or narrowed distal region 1122a of the pump body. The guidewire housing member 112 may be substantially radially constrained and centered relative to the pump body 120 by its interaction with the support member 116. In some variations, the volume displacement member 130 may have a narrowed distal neck 134 that engages with the support member 116 (e.g., coupled end-to-end with the support member, arranged within the lumen of the support member 116, arranged circumferentially around the support member 116, etc.) so as to similarly be substantially radially constrained and centered relative to the pump body 120.

### E. Catheter

As described above, the circulatory assist device 100 may include a catheter 110 having one or more lumens extending longitudinally therethrough. For example, the catheter may include an inflation lumen and/or a guidewire lumen. The inflation lumen may, for example, be in fluidic communication with the volume displacement member 130 for enabling the expansion and/or contraction of the volume displacement member 130. The guidewire lumen may be configured to receive a guidewire and/or guidewire housing member 112. Furthermore, at least a portion of the catheter 110 may function as a shaft onto which the volume displacement member 130 may be mounted (e.g., balloon shaft).

### 1. Catheter Coupling to Support

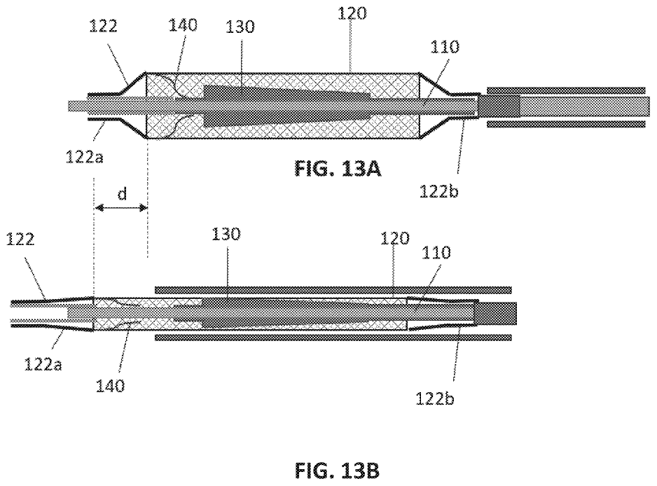

In some variations, the catheter 110 may be coupled to one end of the pump body 120 (e.g., the support 122). For example, in some variations, the catheter 110 may be coupled to a proximal end of the support 122 and freely movable at the distal end of the support 122. For example, FIGS. 13A and 13B are schematic illustrations of an example circulatory assist device 1300, which may be similar to circulatory assist device 100 in many ways, except as described below. For example, the circulatory assist device 1300 may include a pump body 120 with a support 122 and a membrane 124 adjacent to the support. A multi-leaflet inlet valve 140 may be coupled to the membrane 124 and arranged near a distal inflow region of the pump body 120 to receive blood into the pump body 120. A volume displacement member 130 (e.g., balloon) may be arranged within the pump body 120 so as to pump received blood out of the pump body. A catheter 110 may be in fluidic communication with the volume displacement member 130 for facilitating cyclical inflation and deflation of the volume displacement member 130. Furthermore, the catheter 110 may be coupled to the support 122 at a proximal support end 122b, while being freely movable relative to the distal support end 122a.

FIGS. 13A and 13B also illustrate the concept of foreshortening by showing the circulatory assist device 1300 in a radially expanded configuration and a low-profile configuration, respectively. When the circulatory assist device 1300 is deployed in the radially expanded configuration (FIG. 13A), the support 122 may be shorter by a distance (d) compared to when the circulatory assist device 1300 is in the low-profile configuration (FIG. 13B). In other words, when the circulatory assist device 1300 is transitioned to the low-profile configuration (FIG. 13B), the support 122 may both narrow and elongate. During such a transition between the radially expanded and low-profile configurations, the free relative movement between (i) the catheter 110 and (ii) the distal support end 122a and valve 140 advantageously helps accommodate the change in length of the support 122. This relative movement thus allows the circulatory assist device 1300 to experience less strain and risk of failure that otherwise may occur as the support 122 changes length if, for example, both the proximal support end 122b and the distal support end 122a were attached to the catheter 110. Furthermore, during the transition to a low-profile configuration, the free movement of the valve 140 away from volume displacement member 130 and/or other components can reduce material stack-up that contributes to bulk, thereby further reducing the diameter of the low-profile configuration (e.g., lowering the crimp profile). In some variations, it may also be advantageous for the catheter 110 to be coupled to the proximal support end 122b to help facilitate retrieval of the circulatory assist device 1300 from the patient. For example, the circulatory assist device 1300 may be removed from the patient by withdrawing (e.g., pulling proximally) on the catheter 110, which may cause the device 1300 to simultaneously elongate and reduce in diameter to a lower profile configuration for easier passage through vasculature.

As described above, the circulatory assist device 1300 illustrated in FIGS. 13A and 13B includes a proximal support end 122b that is fixed to the catheter 110 and a distal support end 122a that is movable relative to the catheter 110. However, in some variations, the catheter 110 may be coupled to the distal support end 122a while being movable relative to the proximal support end 122b, which similarly may accommodate the change in length of the support 122 with respect to foreshortening. The connection between the support 122 (e.g., the proximal support end 122b or the distal support end 122a) and the catheter 110 may be strong and durable enough to withstand repetitive stress throughout operation of the circulatory assist device.

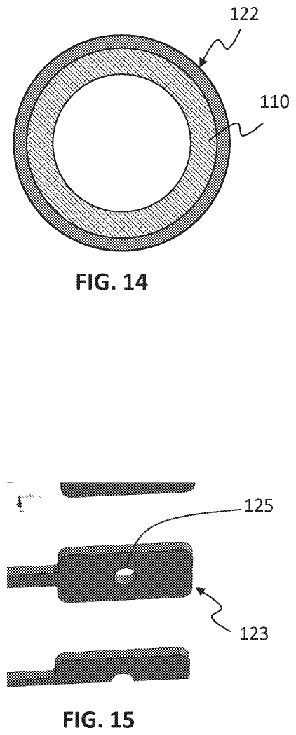

The support 122 may be coupled to the catheter 110 in any one or more various suitable manners. For example, in some variations the support 122 (e.g., the proximal support end 122b or the distal support end 122a) may be coupled to the catheter 110 via a suitable adhesive, such as a UV cured epoxy. The support 122 and/or the catheter 110 may, in some variations include one or more features to enhance or improve the bond strength of the adhesive, particularly in variations in which the support 122 includes a metal material (e.g., nitinol) and the surface of the catheter 110 includes a polymer material, since coupling metal and polymer surfaces effectively may be challenging. For example, as shown in FIG. 14, at the coupling region (e.g., the proximal support end 122b or the distal support end 122a (not labeled in FIG. 14)), the inner diameter of the support 122 may substantially match the outer diameter of the catheter 110 (and/or the curvature of the inner surface of the support 122 may substantially match the curvature of the outer surface of the catheter 110), in order to increase the bond surface area between the inner surface of the support 122 and the outer surface of the catheter 110.

As another example, the support 122 may additionally or alternatively include one or more openings through which adhesive may flow to bond with the catheter 110. As shown in the example of FIG. 15, a support may include one or more tabbed struts 123 having a contact surface defining at least one aperture 123. The tabbed struts 123 may, for example, extend proximally from a proximal support end 122b, or extend distally from a distal support end 122a. In some variations, adhesive may flow through the aperture(s) 123 between an outer surface of the tabbed strut 123 and a polymer surface of the catheter 110 adjacent an inner surface of the tabbed strut 123. Additionally or alternatively, adhesive may flow between and/or over the tabbed struts 123. In some variations, an outer band (not shown) may additionally encircle the tabbed struts 123 to cover the coupling of the support 122 and the catheter 110. Although the tabbed struts 123 shown in FIG. 15 are shown as having substantially rectangular tabs each with a single aperture 125 centered in the tabbed strut, it should be understood that the tabbed struts 123 may have any suitable tab shape (e.g., square, triangular, circular, etc.) and/or have any suitable number of apertures 125 arranged in any suitable manner (e.g., one, two, three or more apertures on a tabbed strut 123).

Additionally or alternatively, the bonding surfaces of the support 122 and/or the catheter 110 may be undergo surface processing to improve bond strength of the adhesive. In some variations, at least a portion of the support 122 (e.g., inner surface of the proximal support end 122b or distal support end 122a) and/or at least a portion of the catheter 110 (e.g., outer surface of the catheter 110) may under chemical priming to improve adhesive performance, such as by applying a silane coupling agent to promote adhesion as a primer (or additionally or alternatively, the silane may be blended with the adhesive itself). Suitable silane coupling agents may include, for example, Dynasylan® AMEO or AMEO-T, though other suitable silane coupling agents may be used. Furthermore, in some variations, the bonding surfaces of the support 122 and/or the catheter 110 may undergo a suitable plasma treatment prior to bonding, to remove any residual organics and increase surface energy of bonding surfaces, thereby improving adhesive strength as well as the flow and/or coverage of the adhesive across the bonding surfaces.

In some variations, the support 122 (e.g., the proximal support end 122b or the distal support end 122a) may be coupled to the catheter 110 via suitable mechanical engagement, in addition to or as an alternative to adhesive. For example, in some variations, the support 122 may mechanically engage with an additional component that is adhesively coupled to the catheter 110, thereby combining mechanical and adhesive techniques for coupling the support 122 and the catheter 110. The additional component may, for example, include a polymer material that is able to more easily bond to a polymer outer surface of the catheter 110 than in a metal-to-polymer interface, thereby enabling a stronger coupling between the support 122 and the catheter 110.

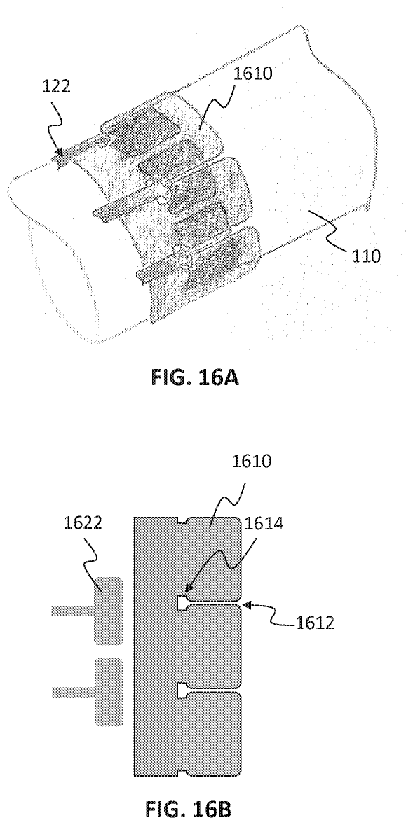

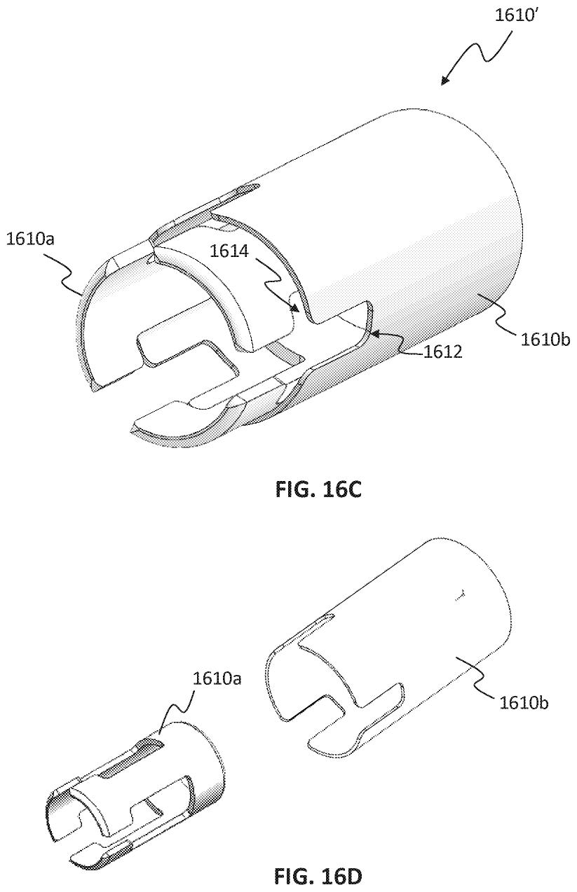

In some variations, the additional component may be a collar that jointly couples to the catheter 110 and the support 122. For example, FIGS. 16A and 16B illustrate an example coupling arrangement in which a collar 1610 is adhesively coupled to the catheter 110 while mechanically engaging with the support 122. The collar 1610 may be coupled to the catheter 110 using any suitable adhesive, similar to that described above. As shown in FIG. 16B, the collar 1610 may, for example, include one or more slots 1612, where each slot 1612 configured to receive and interlock with an engagement feature on the support 122. For example, the proximal support end 122b may include tabbed struts 1622 (e.g., “T”-shaped struts) that interlock with a longitudinal slot 1612 and/or on the collar 1610. In this example, when the catheter 110 is pulled in a proximal direction (to the right in the perspective shown in FIGS. 16A and 16B), the collar 1610 and tabbed struts 1622 on the support 122 resist their separation due to their interlocking engagement, thereby causing the support 122 to be pulled in tandem with the catheter 110. Conversely, in some variations, a similar coupling arrangement with a collar and tabbed struts on the distal support end 122a may enable the catheter 110 to couple to the distal end of the support 122. Although the tabbed struts 1622 shown in FIG. 16B are shown as having substantially rectangular tabs, it should be understood that the tabbed struts 1622 may have tabs of any suitable shape (e.g., square, triangular, circular, etc.). Furthermore, although the collar 1610 shown in FIG. 16A is fully circumferential with a particular shape of slots 1612 and 1614, it should be understood that in some variations the additional component that jointly couples to the catheter 110 and the support 122 may be partially circumferential around the catheter 110, and/or the coupling arrangement may include multiple such additional components distributed around the circumference of the catheter 110.

FIGS. 16C and 16D illustrate another example coupling arrangement similar to that shown in FIGS. 16A and 16B, except that the coupling arrangement of FIGS. 16C and 16D includes an additional component in the form of a collar 1610′ with multiple collar portions (e.g., 1610a and 1610b), shown assembled in FIG. 16C and in an exploded view in FIG. 16D. The collar 1610′ may be coupled to the catheter 110 (not shown), and the multiple collar portions may be coupled to one another using any suitable adhesive, similar to that described above. Like the collar 1610, the collar 1610′ may define (collectively among the multiple collar portions) one or more longitudinal slots 1612 and/or one or more radial slots 1614 configured to interlock with tabbed struts of the support 122 (not shown). Accordingly, when the catheter 110 is pulled in a proximal direction (to the right in the perspective shown FIGS. 16C and 16D), the collar 1610′ and tabbed struts on the support 122 resist their separation due to their interlocking engagement, thereby causing the support 122 to be pulled in tandem with the catheter 110. Furthermore, although the collar 1610′ shown in FIG. 16C is fully circumferential with a particular shape of slots 1612 and 1614, it should be understood that in some variations the additional component that jointly couples to the catheter 110 and the support 122 may be partially circumferential around the catheter 110, and/or the coupling arrangement may include multiple such additional components distributed around the circumference of the catheter 110.

In some variations, the catheter 110 may define one or more engagement features for mechanically coupling with one or more corresponding engagement features on the support 122 (e.g., the proximal support end 122b or the distal support end 122a). For example, the catheter 110 may include one or more recesses or projections configured to receive a corresponding engagement feature on the support 122, and/or the catheter 110 and support 122 may include any suitable interlocking components. As another example, the catheter 110 and the support 122 may couple to one another via an additional locking component (e.g., pin). Illustrative examples of mechanical coupling between the catheter 110 and the support 122 are described in further detail below. In some variations, the inner diameter of the portion of the support 122 that couples to the catheter 110 may be slightly undersized relative to the outer diameter of the catheter 110, to thereby improve the interlock or interference fit between the support 122 and the catheter 110.

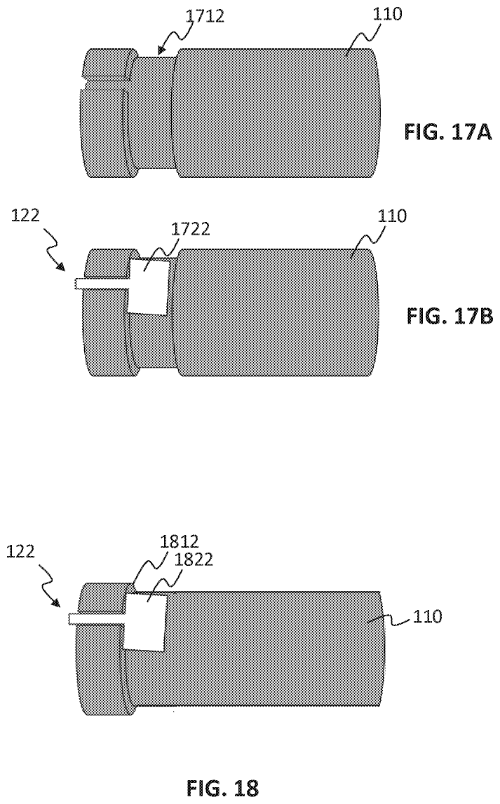

For example, the outer surface of the catheter 110 may include a recess that receives and retains a corresponding engagement feature on the support 122. In some variations, the engagement feature on the support 122 may additionally be coupled to the recess via adhesive (similar to that described above), and/or via a collar (e.g., polymer collar, similar to collar 1610 or other suitable additional component as described above with respect to FIGS. 16A and 16B). In the coupling arrangement shown in FIGS. 17A and 17B, for example, the outer surface of the catheter 110 may include a recessed channel 1712 that is configured to retain an engagement feature on the support 122. For example, the proximal support end 122b may include one or more tabbed struts 1722 (e.g., “T”-shaped struts) that may be received and retained in the channel 1712. In this example, when the catheter 110 is pulled in a proximal direction (to the right in the perspective shown in FIGS. 17A and 17B), the catheter 110 and the tabbed struts 1722 on the support 122 resist their separation due to the retention of the tabbed struts 1722 in the catheter 110, thereby causing the support 122 to be pulled in tandem with the catheter 110. Conversely, in some variations, a similar coupling arrangement with a recess and an engagement feature on the distal support end 122a may enable the catheter 110 to couple to the distal end of the support 122. Although the tabbed struts 1722 shown in FIG. 17B are shown as having substantially rectangular tabs, it should be understood that the tabbed struts 1622 may have tabs of any suitable shape (e.g., square, triangular, circular, etc.). Similarly, while the channel 1712 is shown as a circular channel extending circumferentially around the catheter 110, in some variations the catheter 110 may have any suitable shape, including a partial ring, or a recess that is complementary to the shape of the tabbed struts 1722. The tabbed struts 1722 may, in some variations, be further coupled to the channel 1712 with adhesive and/or mechanically coupled to a polymer collar that couples to the catheter 110 with adhesive. For example, the polymer collar (not shown) may be heat shrunk or cold shrunk over the tabbed struts 1722 to help compress and retain the tabbed struts 1722 in the channel 1712.

As another example, the outer surface of the catheter 110 may include a recessed or necked region configured to engage with an engagement feature (e.g. narrowed profile, tabbed struts, etc.) of the catheter 110. For example, as shown in FIG. 18, a distal end of the catheter 110 may include a necked (e.g., stepped or tapered) region of a smaller outer diameter, thereby forming a stop 1812 against which the support 122 can engage. For example, similar to that shown in FIGS. 17A and 17B, the distal support end of the support 122 may include one or more tabbed struts 1822 configured to abut against the stop 1812. In this example, when the catheter 110 is pulled in a proximal direction (to the right in the perspective shown in FIG. 18), the catheter 110 and the distal support end 122a resist their separation due to the engagement between the tabbed struts 1822 and the stop 1812, thereby causing the support 122 to be pulled in tandem with the catheter 110. Conversely, in some variations, a similar coupling arrangement with a recess and an engagement feature on the distal support end 122a may enable the catheter 110 to couple to the distal end of the support 122. Although the tabbed struts 1822 shown in FIG. 18 are shown as having substantially rectangular tabs, it should be understood that the tabbed struts 1822 may have tabs of any suitable shape (e.g., square, triangular, circular, etc.). Similarly, while the stop 1812 is shown as extending circumferentially around the catheter 110, in some variations the stop 1812 may have any suitable shape, including a partial ring, or a recess that is complementary to the shape of the tabbed struts 1822. The distal support end 122a (e.g., tabbed struts 1822) may, in some variations, be further coupled to the necked region of the catheter 110 with adhesive and/or mechanically coupled to a polymer collar that couples to the catheter 110 with adhesive. For example, the polymer collar (not shown) may be heat shrunk or cold shrunk over the distal support end 122a and the necked region of the catheter 110, to help compress and retain the distal support end 122a over the catheter 110.

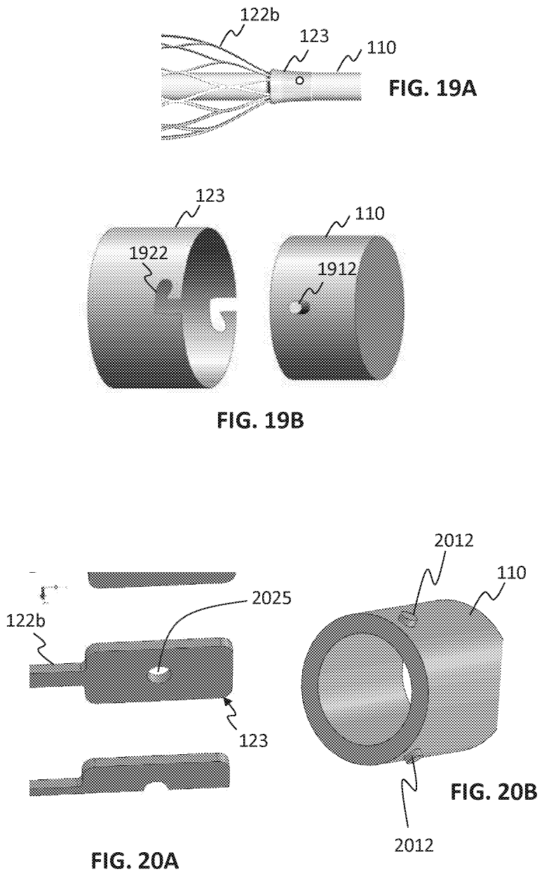

In some variations, one or both of the catheter 110 and the support 122 may include suitable interlocking components. For example, as shown in FIGS. 19A and 19B, the proximal support end 122b may include a proximal band 123 with a slot 1922. The proximal band 123 may, for example, be integrally formed with the support 122 (e.g., the support 122 may be laser cut from a tube, such as a nitinol tube). The slot 1922 may be configured to receive and retain a projection 1912 extending radially outward from the catheter 110. The slot 1922 can have a circumferential or angled component (e.g., “L”-shaped slot similar to that on a bayonet mount) to help resist longitudinal separation between the catheter 110 and the support 122 when the projection 1912 is received in the slot 1922. Additionally or alternatively, the slot 1922 may be configured to engage with the projection 1912 in a snap-fit manner. Accordingly, when the catheter 110 is pulled in a proximal direction (to the right in the perspective shown in FIG. 19A), the catheter 110 and the support 122 resist their separation due to the retention of the projection 1912 in the slot 1922, thereby causing the support 122 to be pulled in tandem with the catheter 110. Conversely, in some variations, a similar coupling arrangement with at least one radially outward projection on the catheter 110 and at least one slot on the distal support end 122a may enable the catheter 110 to couple to the distal end of the support 122. In some variations, in the coupling arrangement between the catheter 110 and the support 122, the support 122 may include a plurality of slots 1922 and/or the catheter 110 may include a plurality of projections 1912 that are circumferentially distributed (e.g., equally circumferentially distributed) around the support 122 and catheter 110, respectively.

Although the example shown in FIGS. 19A and 19B includes a support 122 with a slot-type engagement feature, in some variations the support 122 may include other kinds of engagement features. For example, as shown in FIGS. 20A and 20B, the proximal support end 122b may include one or more tabbed struts 123 including an aperture 2025 configured to receive and engage with a projection 2012 that extends radially outward from the catheter 110. Additionally, in some variations the aperture 2025 may be configured to engage with a corresponding projection 2012 in a snap-fit manner. Accordingly, when the catheter 110 is pulled in a proximal direction (to the right in the perspective shown in FIGS. 20A and 20B), the catheter 110 and the support 122 resist their separation due to the retention of the projection 2012 in the aperture 2025, thereby causing the support 122 to be pulled in tandem with the catheter 110. Conversely, in some variations, a similar coupling arrangement with at least one radially outward projection on the catheter 110 and at least one aperture on the distal support end 122a may enable the catheter 110 to couple to the distal end of the support 122. In some variations, in the coupling arrangement between the catheter 110 and the support 122, the support 122 may include a plurality of apertures 2025 and/or the catheter 110 may include a plurality of projections 2012 that are circumferentially distributed (e.g., equally circumferentially distributed) around the support 122 and catheter 110, respectively.

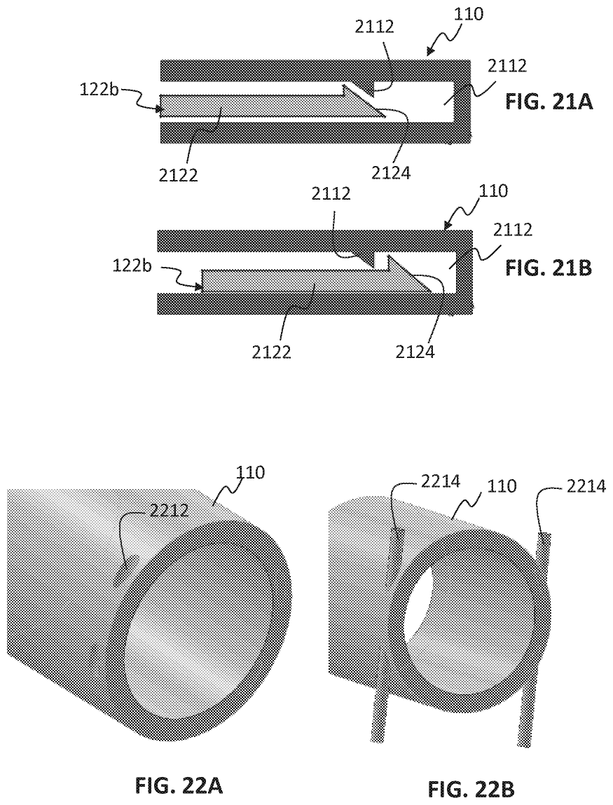

In some variations, the support 122 and/or the catheter 110 may include a receptacle configured to receive a longitudinally-extending engagement feature for coupling. The receptacle and/or longitudinally-extending engagement feature may include a lock element such as a ratchet-like tooth configured to help retain the longitudinally-extending engagement feature in the receptacle. For example, as shown in FIGS. 21A and 21B, the catheter 110 may include a female receptacle 2112 configured to receive a male engagement feature 2122 on the proximal support end 122b, such as a strut. The receptacle 2112 may include a locking tooth 2112 that is angled to permit insertion of the male engagement feature 2122, but that abuts a corresponding locking tooth 2024 to prevent removal of the male engagement feature 2122 from the receptacle 2112. In some variations, the receptacle 2112 and the male engagement feature 2122 may engage in a snap-fit manner. Accordingly, when the catheter 110 is pulled in a proximal direction (to the right in the perspective shown in FIGS. 21A and 21B,), the catheter 110 and the support 122 resist their separation due to the retention of the male engagement feature 2122 in the receptacle 2112, thereby causing the support 122 to be pulled in tandem with the catheter 110. Conversely, in some variations, a similar coupling arrangement with at least one radially outward projection on the catheter 110 and at least one aperture on the distal support end 122a may enable the catheter 110 to couple to the distal end of the support 122. In some variations, the receptacle 2112 may be annular (and may, for example, have an inner annular rib with a locking tooth profile) and configured to receive and retain multiple circumferentially distributed male engagement features 2122. However, in some variations the catheter 110 may include multiple receptacles 2112, each of which is configured to receive and retain a respective male engagement feature 2122. Furthermore, it should be understood that additionally or alternatively, in some variations, the support 122 may include a receptacle, and the catheter 110 may include a male engagement feature.

In some variations, the catheter 110 and the support 122 may couple to one another via an additional locking component (e.g., pin). For example, as shown in FIGS. 22A and 22B, the catheter 110 may include pin engagement features 2212 (e.g., holes or rails) configured to receive a pin 2214. The support (e.g., a distal support end, or a proximal support end such as a proximal band similar to proximal band 123 shown in FIG. 19) may include holes that may be aligned with the pin engagement features 2212, such that pins 2214 may be inserted through both the pin engagement features 2212 and the holes in the support, so as trap or interlock the support around the catheter 110. Accordingly, when the catheter 110 is pulled in a proximal direction, the catheter 110 and the support 122 resist their separation due to the locking function of the pin, thereby causing the support 122 to be pulled in tandem with the catheter 110. The pins 2212 may be straight as shown in FIG. 22B, or may be curved to follow the curvature of the catheter 110 (which may, for example, help reduce risk of tissue trauma and/or help fix the position of the pin 2212). Furthermore, while in some variations the pins 2212 may be separate components from the support 122 and the catheter 110, in some variations any one or more pins 2212 may be integrally formed with the support 122 or the catheter 110.

Although various examples of mechanical and/or adhesive coupling between the catheter 110 and support 122 are described above and shown, the catheter 110 and support 122 may additionally or alternatively be coupled in any suitable manner. For example, the catheter 110 and support 122 may be coupled to one another with a suitable threaded interface, or a suitable snap fit interface (e.g., annular ring that snap fits with an annular channel). Furthermore, while the figures may show one component of the support 122 and the catheter 110 as including a male feature while the other component includes a female feature that couples to the male feature, it should be understood that in some variations the male and female features may be swapped between the support 122 and the catheter 110.

### 2. Catheter Reinforcement

In some variations, the catheter 110 may be reinforced to increase axial strength (e.g., tensile strength, column strength) and/or increase stiffness to better withstand forces such as during pushing and/or pulling of the catheter 110 during positioning and/or operation of the circulatory assist device. The catheter 110 may be reinforced, for example, with one or more reinforcement members along the catheter 110 wall.

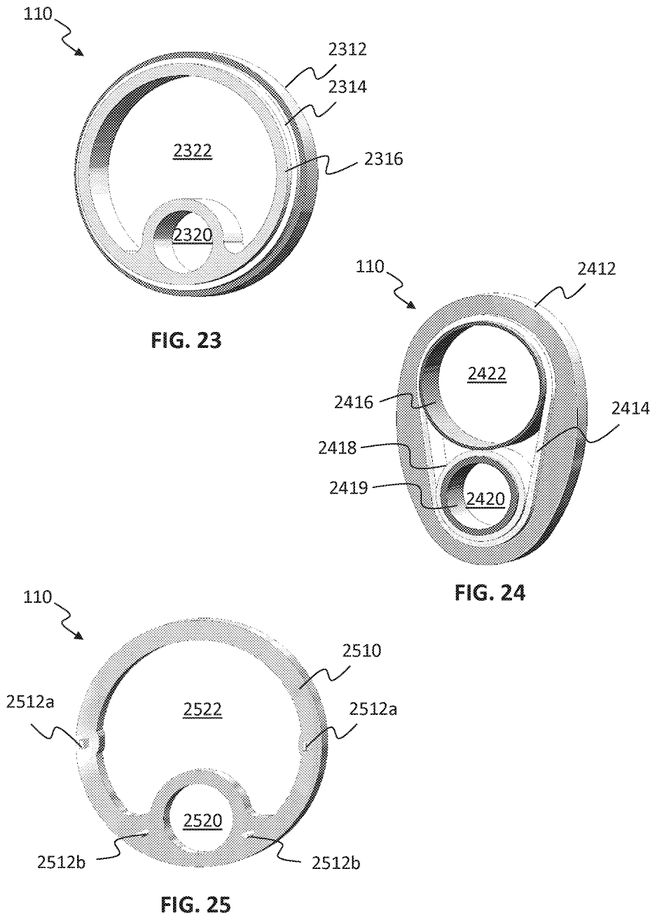

In some variations, one or more reinforcement members may include a hypotube including a material (e.g., metal) with suitable high axial strength. The hypotube may be arranged as a layer in the wall of the catheter 110. For example, as shown in the cross-sectional view of FIG. 23, an example catheter 110 may include a hypotube 2314 arranged between an outer jacket 2312 and an inner member 2316. The inner member 2316 may, for example, include multiple lumens such as an inflation lumen 2322 configured to convey a fluid to and/or from the volume displacement member 130 for operating the volume displacement member 130 between the expansion and contraction phases, and/or a guidewire lumen 2320.

In some variations, the hypotube may extend only along a portion of the catheter 110 (e.g., a distal portion of the catheter near the pump body), or may extend along the entire length of the catheter 110 (e.g., proximally to an external handle that is coupled to the catheter 110). In variations in which both the support 122 and the hypotube are made of metal, the hypotube may also be directly coupled to the support 122 in a metal-to-metal connection. For example, a proximal support end 122b (not shown in FIG. 23) may be coupled mechanically and/or with adhesive to a portion of the hypotube 2314, which may be exposed (e.g., the outer jacket 2312 not covering the hypotube 2314).

As another example, as shown in the cross-sectional view of FIG. 24, an example catheter 110 may include at least one reinforcement member in the form of a braided, coiled, or mesh member 2414 that is arranged between an outer jacket 2412 and one or more inner members. The inner members may, for example, include a first inner member 2416 (e.g., PTFE liner) defining an inflation lumen 2422 configured to convey a fluid to and/or from the volume displacement member 130 for operating the volume displacement member 130 between the expansion and contraction phases. The inner members may further include a second inner member 2419 (e.g., platinum tube with PTFE liner) defining a guidewire lumen. The second inner member 2419 may furthermore have an outer polymer lining 2418 (e.g., nylon). In some variations, the braided or coiled member 2414 may include a suitable metal material (e.g., nitinol) with suitably high axial strength, and/or a polymer material such as a polymer outer skin. As shown in FIG. 24, the braided, coiled, or mesh member 2414 may be arranged around the first inner member 2416 and second inner member 2419, and the outer jacket may be arranged around the member 2414.

In some variations, the member 2414 may extend only along a portion of the catheter 110 (e.g., a distal portion of the catheter near the pump body), or may extend along the entire length of the catheter 110 (e.g., proximally to an external handle that is coupled to the catheter 110). In some variations, the support 122 may be coupled mechanically and/or with adhesive to the member 2414.

As another example, as shown in the cross-sectional view of FIG. 25, an example catheter 110 may include one or more reinforcement members in the form of wire support members 2512a, 2512b embedded in a wall of the catheter 110, such as embedded in a multi-lumen structure 2510. The wire support members may include a suitable metal material, such as stainless steel or nitinol. The multi-lumen structure 2510 may, for example, include an inflation lumen 2522 configured to convey a fluid to and/or from the volume displacement member 130 for operating the volume displacement member 130 between the expansion and contraction phases, and a guide wire lumen 2520. The wire support members may, for example, include a flat wire 2512a and/or a round wire 2512b. In some variations, the wire support members 2512a, 2512b may be distributed around the circumference of the multi-lumen structure 2510 (e.g., equally circumferentially distributed). The wire support members 2512a, 2512b may extend only along a portion of the catheter 110 (e.g., a distal portion of the catheter near the pump body, or near a connection region where the catheter 110 couples to the support 122), or may extend along the entire length of the catheter 110 (e.g., proximally to an external handle that is coupled to the catheter 110).

Additionally or alternatively, in some variations, it may be advantageous to increase the stiffness of the catheter 110 in portions near (e.g., adjacent to) the connection between the catheter 110 and the support 122, such that when the catheter 110 is manipulated (e.g., pushed, pulled), the strain around the connection with the support 122 is more evenly distributed, thereby lowering risk of failure of the connection. In some variations, stiffness of the catheter 110 may be increased by selecting a stiffer catheter material in and/or adjacent the connection location, by increasing the thickness of the material in and/or adjacent the connection location, and/or adding suitable reinforcement structures and/or other strain relief around the connection location (e.g., collar around the support 122 and catheter 110 at the connection location).

### F. Example Configurations

FIG. 7 is a schematic illustration of an example circulatory assist device 700 that is similar to circulatory assist device 100, except with further details described herein. For example, the circulatory assist device 700 may include a pump body 120 with a support and a membrane adjacent to the support. A multi-leaflet inlet valve 140 may be arranged near a distal inflow region of the pump body 120 to receive blood into the pump body 120. For example, the inlet valve 140 may be mounted to the support and/or membrane of the pump body. A volume displacement member 130 (e.g., balloon) may be arranged within the pump body 120 and configured for cyclical inflation and deflation so as to pump received blood out of the pump body.

The support may include a necked distal region 122a and a necked proximal region 122b. The circulatory assist device 700 may further include a support member 116 arranged in and coupled to the necked distal region 122a. The support member 116 may receive a guidewire 111 and/or guidewire housing member 112 so as to help center the guidewire 111 and/or guidewire housing member 112 relative to the necked distal region 112a, and/or help provide structural support to the guidewire 111 and/or guidewire housing member 112. The centered guidewire 111 and/or guidewire housing member 112 may also pass between the leaflets of the inlet valve 140 (including when the inlet valve 140 is in the closed state), which may help center the inlet valve 140 (thereby centering the fluid passing therethrough) that is mounted in the pump body 120. The centered guidewire 111 and/or guidewire housing member 112 may also pass longitudinally through the center of the volume displacement member 130 (e.g., including through necked distal region 134 of the volume displacement member 130 and necked proximal region 136 of the volume displacement member 130), which may help center the volume displacement member 130 within the pump body 120. As shown in FIG. 7, the support member 116 additionally may be coupled to a proximal end 134 of the volume displacement member 130. The proximal end 134 of the volume displacement member 130 may also pass through an aperture of the inlet valve 140 (e.g., similar to that described above with respect to FIG. 6O).

FIG. 8 is a schematic illustration of an example circulatory assist device 800 that is similar to circulatory assist device 700, except that the support member 116 is not coupled to the proximal end 134 of the volume displacement member 130. However, like in circulatory assist device 700, a support member 116 may be configured to help center the guidewire 111 and/or guidewire housing member 112 relative to the pump body 120, and/or help provide structural support to the guidewire 111 and/or guidewire housing member 112. Furthermore, the guidewire 111 and/or guidewire housing member 112 may be configured to help center the volume displacement member 130 relative to the pump body 120.

FIG. 8A is a schematic illustration of an example circulatory assist device 800′ that is similar to circulatory assist device 800, except that the support member 116 extends through the opening of the inlet valve 140. The support member 116 may or may not additionally be coupled to a proximal end 134 of the volume displacement member 130. However, like in circulatory assist device 800, the support member 116 may be configured to help center the guidewire 111 and/or guidewire housing member 112 relative to the pump body 120, and/or help provide structural support to the guidewire 111 and/or guidewire housing member 112. In some variations, the support member 116 may, for example, extend past the free leaflet edge of the inlet valve 140 by at least between about 1 mm and about 15 mm, or at least 5 mm, or at least 10 mm.

FIGS. 9A and 9B are schematic illustrations of an example circulatory assist device 900 that is similar to circulatory assist device 700 described above with respect to FIG. 8, except that the volume displacement member 130 includes one or more radial protrusions 132 (e.g., ribs, fins, etc.). As shown in the cross-sectional view of FIG. 9B, the radial protrusions 132 may be circumferentially distributed around the volume displacement member 130, and may be configured to engage with the inner surface of the pump body 120 (e.g., support and/or membrane) so as to center the volume displacement member 130 within the pump body 120 at least during the expansion phase of the volume displacement member 130 (e.g., inflation phase). Additionally or alternatively, the volume displacement member 130 may include a narrowed or necked distal region 134 that is received within a central aperture of the inlet valve 140, thereby centering the volume displacement member 130 relative to the inlet valve 140.

FIGS. 10A and 10B are schematic illustrations of an example circulatory assist device 1000 that is similar to circulatory assist device 800 described above with respect to FIG. 8, except as described below. As shown in the side view of FIG. 10A, the guidewire housing member 112 may include a coaptation region 113 that interacts with the leaflets of the inlet valve 140 when the inlet valve 140 is in its closed state. The coaptation region 113 may, for example, have a cross-sectional profile that complements or corresponds to the coaptation pattern between adjacent leaflets. For example, in some variations in which the coaptation region 113 is configured to interface with leaflets of a tricuspid valve, the coaptation region 113 may have the profile of a curvilinear triangle (e.g., deltoid curve). The profiled coaptation region 113 may help improve coaptation contact, thereby reducing backflow of the inlet valve 140. Additionally, the profiled coaptation region 113 may help to reduce contact pressure on the leaflets themselves, thereby improving durability of the valve leaflets. In some variations, the coaptation region 113 may also include a soft material to improve the sealing during coaptation contact and/or further reduce wear and tear on the valve leaflets engaging the coaptation region 113. Furthermore, in some variations, the coaptation region may help to center the guidewire housing member 112 (and/or guidewire 111) within the valve leaflets.

FIGS. 11A and 11B are schematic illustrations of an example circulatory assist device 1100 with the inlet valve 140 in a closed state and an open state, respectively. The circulatory assist device 1100 may be similar to the circulatory assist device 800 described above with respect to FIG. 8, except as described below. In the circulatory assist device 1100, the pump body 120 may, in an expanded valve region 128 (e.g., in or overlapping with the inflow region 120a), have an outwardly projecting wall so as to be enlarged relative to other region(s) of the pump body 120. The outwardly projecting wall may provide a space (e.g., annular space) into which the leaflets of the inlet valve 140 may retreat when the inlet valve 140 is in the open state (FIG. 11B). This may, for example, help avoid reduction of flow diameter into the pump body, and help maintain substantially consistent flow diameter for fluid entering and passing through the pump body 120.

FIG. 12 is a schematic illustration of an example circulatory assist device 1200 similar to circulatory assist device 800 described above with respect to FIG. 8, except as described below. In the circulatory assist device 1200, the pump body 120 may, in a narrowed neck region 129 (e.g., in or overlapping with the inflow region 120a), have an inwardly projecting wall so as to be narrowed relative to other region(s) of the pump body 120. The inwardly projecting wall may reduce the amount of material that is crimped when the device is collapsed to a low-profile configuration, thereby helping the crimped profile of the neck region 129 to substantially match the crimped profile of the rest of the device, and allow for a consistent crimped device diameter along the length of the pump body.

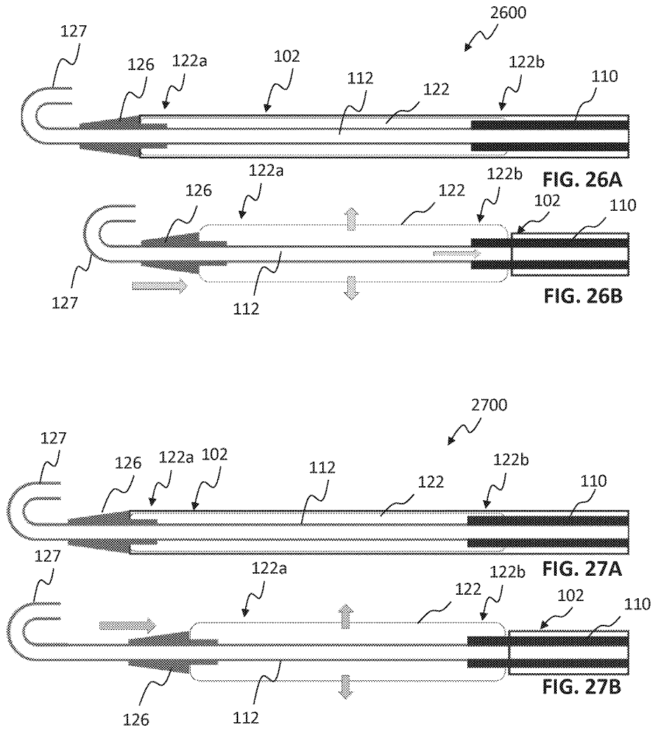

FIGS. 26A-29B are schematic illustrations of various example configurations for enabling a distal end of the support 122 to move relative to the catheter 110 (also referred to below as a balloon shaft), while a proximal end of the support 122 is coupled or fixed relative to the catheter 110. As described above, allowing the distal end of the support 122 to move relative to the catheter 110 while the proximal end of the support 122 is coupled to the catheter 110 may accommodate the foreshortening effect of the support 122.

FIGS. 26A and 26B illustrate an example configuration of a circulatory assist device 2600 in a low profile configuration and a radially expanded configuration, respectively. Various elements of the device 2600 (e.g., volume displacement member, membrane, etc.) are not shown for clarity purposes. In FIG. 26A, the device 2600 is radially constrained at least in part by an outer sheath 102, which can be retracted proximally to allow the device 2500 to radially expand to the configuration shown in FIG. 26B (e.g., through self-expansion). The circulatory assist device 2600 may include a balloon shaft 110 on which the volume displacement member (not shown) may be mounted, and a guidewire housing member 112 configured to receive a guidewire (not shown). The support 122 of the pump body may be arranged around the guidewire housing member 112, and has a proximal support end 122b coupled to the balloon shaft 110 (e.g., in any of the manners described herein), and a distal support end 122a coupled to the guidewire housing member 112. A distal tip 126 (e.g., screw connector) and pigtail 127 may be arranged at a distal end of the guidewire housing member 112.

In the device 2600, the guidewire housing member 112 is moveable within the balloon shaft 110 (e.g., in a guidewire lumen defined in the balloon shaft 110). The pigtail 127 and distal tip 126 may be coupled to the guidewire housing member 112 and/or the distal support end 122a, but movable relative to the balloon shaft 110. Accordingly, when the support 122 radially expands and foreshortens proximally (FIG. 26B), the pigtail 127, the distal tip 126, and the distal support end 122a may move proximally in accordance with the foreshortening effect, thereby reducing strain in, for example, the support 122. In some variations, an additional inner support member (not shown), such as a polymer tube (e.g., polyimide), may be arranged inside the guidewire housing member 112 to help provide axial support and reduce kinking of the guidewire housing member 112 when the guidewire housing member 112 moves.

FIGS. 27A and 27B illustrate an example configuration of a circulatory assist device 2700 in a low profile configuration and a radially expanded configuration, respectively. Various elements of the device 2700 (e.g., volume displacement member, membrane, etc.) are not shown for clarity purposes. The device 2700 may be similar to the device 2600 described above with respect to FIGS. 26A and 26B, except as described below. For example, in the device 2700, the distal tip 126 is coupled to the distal support end 122a and/or otherwise moves in tandem with the distal support end 122a along the guidewire housing member 112 when the distal support end 122a moves proximally in accordance with the foreshortening effect. The guidewire housing member 112 and pigtail 127 may move independently from the distal tip 126 and distal support end 122a, such that they may remain substantially stationary in place during foreshortening of the support 122.

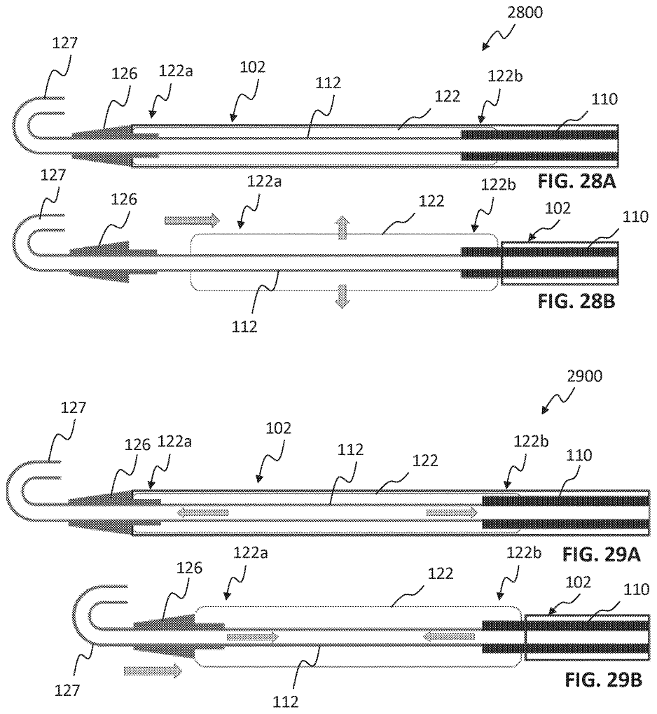

FIGS. 28A and 28B illustrate an example configuration of a circulatory assist device 2800 in a low profile configuration and a radially expanded configuration, respectively. Various elements of the device 2800 (e.g., volume displacement member, membrane, etc.) are not shown for clarity purposes. The device 2800 may be similar to the device 2600 described above with respect to FIGS. 26A and 26B, except as described below. For example, in the device 2800, the distal support end 122a may move independently of the guidewire housing member 122, the connector 126, and the pigtail 127. As such, the guidewire housing member 122, the connector 126, and the pigtail 127 may remain substantially stationary in place during foreshortening of the support 122. During such foreshortening, the support 122 may slide proximally along the guidewire housing member 122.

FIGS. 29A and 29B illustrate an example configuration of a circulatory assist device 2700 in a low profile configuration and a radially expanded configuration, respectively. Various elements of the device 2900 (e.g., volume displacement member, membrane, etc.) are not shown for clarity purposes. The device 2900 may be similar to the device 2600 described above with respect to FIGS. 26A and 26B, except as described below. For example, like in the device 2600, in the device 2900 the support 122 of the pump body may be arranged around the guidewire housing member 112, and have a proximal support end 122b coupled to the balloon shaft 110 (e.g., in any of the manners described herein) and a distal support end 122a coupled to the guidewire housing member 112. However, in the device 2900, a proximal end of the guidewire housing member 112 may be fixed relative to the balloon shaft 110 (instead of being moveable within a guidewire lumen of the balloon shaft 110), thereby fixing the proximal support end 122b relative to the guidewire housing member 112. Accordingly, the distal and proximal ends of the guidewire housing member 112 may be fixed relative to the distal support end 122a and the proximal support end 122b, respectively.

Furthermore, in the device 2900, the guidewire housing member 112 may include a stretchable material (e.g., silicone). During a crimping process in which the pump body is radially collapsed around the guidewire housing member 112 and radially constrained by the outer sheath 102, the support 122 may elongate and longitudinally stretch the guidewire housing member 112 (since the ends of the guidewire housing member 112 are fixed relative to the ends of the support 122). When the outer sheath 102 is retracted proximally, the guidewire housing member 112 may shorten in length in tandem with the support 122 as the support 122 foreshortens proximally. Furthermore, the distal tip 126 and the pigtail 127 may also move proximally in tandem with the support 122 as the support 122 foreshortens proximally.

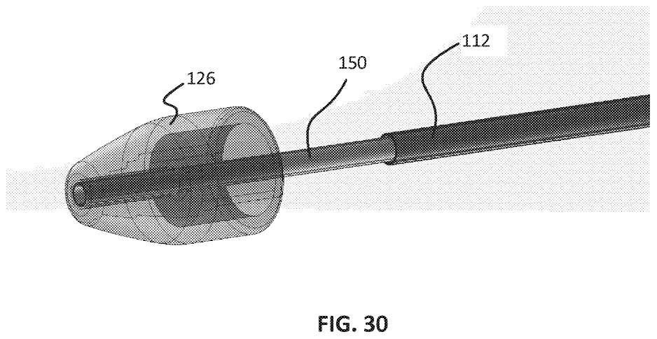

In some variations, the guidewire housing member 112 may omit pigtail 127 at its distal end, and instead include a distal tip 126 that is coupled to an inner member that is configured to move within the guidewire housing member 112. For example, FIG. 30 illustrates an example arrangement with a guidewire housing member 112 configured to receive an inner member 150 therein. A distal tip 126 (e.g., screw connector) may be coupled to a distal end of the inner member 150 (e.g., with adhesive, mechanical engagement, etc.), and a guidewire 111 may be received within a lumen extending through the distal tip 126 and/or the inner member 150. The inner member 150 may, for example, move relative to the guidewire housing member 112 during crimping of the circulatory assist device. The arrangement shown in FIG. 30 with the inner member 150 in lieu of pigtail 127 may, for example, be incorporated into any of the example circulatory assist device configurations described herein (e.g., with respect to FIGS. 26A-29B).

In some variations, only a portion of the guidewire housing member 112 may include a stretchable material. For example, segment(s) of the guidewire housing member 112 outside of the volume displacement member (not shown) may include a stretchable material, while segment(s) of the guidewire housing member 112 coincident with the length of the volume displacement member may be more rigid than the stretchable segments. For example, in some variations the guidewire housing member 112 may include a first portion (at least partially coincident with the volume displacement member) with a first material and a second portion (e.g., distal to the volume displacement member) with a second material, where the first material is more rigid than the second material. The first material may, for example, be overmolded with the second material (or vice-versa) to form a guidewire housing member 112 with a stretchable portion.

Although example pump device configurations are described above with reference to each of FIGS. 1A-31B. it should be understood that in some variations, one or more various features of these examples may be combined in any suitable manner. For example, although a volume displacement member 130 with radial protrusions 132 is described primarily with respect to the variation shown in FIGS. 9A and 9B, it should be understood that the same or similar volume displacement member 130 with radial protrusions 132 may be combined with any of the example device configurations shown in FIGS. 1A-31B. As another example, although a guidewire housing member 112 with a profiled coaptation region is described primarily with respect to the variation shown in FIGS. 10A and 10B, it should be understood that the same or similar guidewire housing member 112 with a profiled coaptation region may be combined with any of the example device configurations shown in FIGS. 1A-31B.

### II. Methods of Operation

In some variations, a method for providing circulatory assistance includes positioning a pump body in the circulatory system of a patient, where the pump body includes a conduit, an inlet valve configured to receive a fluid along a longitudinal flow axis of the conduit, a volume displacement member arranged in the conduit and operable in an expansion phase and a contraction phase. In some variations, the pump body may be substantially similar to that described herein (e.g., pump body 120, such as with respect to FIG. 1A, FIGS. 4A and 4B, FIG. 7, FIG. 8, FIGS. 9A and 9B, FIGS. 10A and 10B, FIGS. 11A and 11B, FIG. 12, and/or FIGS. 13A and 13B). The method may further include cyclically operating the volume displacement member in the expansion phase and the contraction phase, and allowing received fluid to exit the conduit through the outlet during both the expansion phase and the contraction phase. Furthermore, in some variations, the method may include allowing the conduit to concurrently receive fluid through the inlet valve and convey fluid through the outlet, including but not limited to during the end of the expansion phase and during the end of the contraction phase.

For example, in some variations (e.g., as shown in FIG. 1B), the method may include positioning at least a portion of the pump body in a left ventricle of the patient. For example, a distal portion of the pump body (e.g., including an inflow region 120a of the pump body) may be positioned in the left ventricle of the patient and a proximal portion of the pump body (e.g., including an outflow region 120c of the pump body) in an ascending aorta of the patient. Accordingly, in this example, the pump body may be positioned through or across the aortic valve of the patient. In some variations, the pump body may be positioned such that the inlet valve of the circulatory assist device is in the left ventricle, below or inferior to the native aortic valve plane. In some variations, the pump body may be positioned such that the inlet valve of the circulatory assist device is in the aorta (e.g., ascending aorta) of the patient, above or superior to the native aortic valve plane.

As another example, in some variations, the method may include positioning the pump body in a blood vessel of the patient (e.g., aorta). Cyclical operation of the volume displacement member in the expansion phase and the contraction phase may, in some variations in which the volume displacement member is a balloon, include cyclically inflating the balloon via a catheter having an inflation lumen.

### CONCLUSION

Although many of the variations are described above with respect to systems, devices, and methods for circulatory assistance, the technology is applicable to other applications and/or other approaches. For example, any of the various constructions, features, and characteristics of the valves described herein may be incorporated in prosthetic valves used in the heart and other body lumens, such as prosthetic aortic, mitral, tricuspid, or pulmonary valves implanted surgically or by transcatheter techniques, including prosthetic valves constructed of polymers, fabrics, or biologic tissues. Valves used in other medical devices, both intracorporeal and extracorporeal, may also incorporate aspects of the valves described above. Moreover, other variations in addition to those described herein are within the scope of the technology. Additionally, several other variations of the technology can have different configurations, components, or procedures than those described herein. A person of ordinary skill in the art, therefore, will accordingly understand that the technology can have other variations with additional elements, or the technology can have other variations without several of the features shown and described above with reference to FIGS. 1A-31B.

The descriptions of variations of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various variations described herein may also be combined to provide further variations.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific variations have been described herein for purposes of illustration, but that various modifications may be made without deviating from the technology. Further, while advantages associated with certain variations of the technology have been described in the context of those embodiments, other embodiments may also exhibit such advantages, and not all variations need necessarily exhibit such advantages to fall within the scope of the technology. Accordingly, the disclosure and associated technology can encompass other variations not expressly shown or described herein.

I/We claim:

1. A blood pump device comprising:
a pump body comprising:
a conduit having an inflow region and an outflow region; and
an inlet valve arranged in the inflow region and comprising a plurality of leaflets,
wherein at least one leaflet comprises a base region comprising a first bending stiffness and an edge region comprising a second bending stiffness, wherein the second bending stiffness is lower than the first bending stiffness.

2. The blood pump device of claim 1, wherein the at least one leaflet comprises an intermediate region between the base region and the edge region comprising a third bending stiffness, wherein the third bending stiffness is between the first bending stiffness and the second bending stiffness.

3. The blood pump device of claim 1 or 2, wherein the at least one leaflet exhibits a linear transition in bending stiffness between the first bending stiffness and the second bending stiffness.

4. The blood pump device of any one of claims 1-3, wherein the at least one leaflet exhibits a stepwise transition in bending stiffness between the first bending stiffness and the second bending stiffness.

5. The blood pump device of any one of claims 1-4, wherein the base region comprises a first leaflet thickness and the edge region comprises a second leaflet thickness, wherein the second leaflet thickness is less than the first leaflet thickness.

6. The blood pump device of claim 5, wherein the second leaflet thickness is between about 45% and about 80% of the first leaflet thickness.

7. The blood pump device of any one of claims 1-6, wherein the base region comprises a first material, and the edge region comprises a second material, wherein the second material has a lower durometer than the first material.

8. The blood pump device of claim 7, wherein the first material has a durometer of greater than 55 D, and the second material has a durometer of lower than 55 D.

9. The blood pump device of any one of claims 1-8, wherein at least one leaflet comprises multiple layers of material.

10. The blood pump device of any one of claims 1-9, wherein the plurality of leaflets are formed from a single continuous membrane.

11. The blood pump device of any one of claims 1-10, wherein the plurality of leaflets are formed from multiple discrete membranes.

12. The blood pump device of any one of claims 1-11, wherein the plurality of leaflets comprises three leaflets.

13. The blood pump device of any one of claims 1-12, wherein the inlet valve has an outer diameter of about 25 mm or less.

14. The blood pump device of any one of claims 1-13, wherein the inlet valve has an outer diameter of about between about 10 mm and about 15 mm.

15. The blood pump device of any one of claims 1-14, wherein the inlet valve in its open state has a valve orifice area between about 15% and about 90% of a maximum cross-sectional area of the conduit, as measured at a location of the inlet valve.

16. The blood pump device of any one of claims 1-15, wherein the inlet valve has a cutoff angle of between about 0 degrees and about 45 degrees.

17. The blood pump device of any one of claims 1-16, further comprising a guidewire extending through the inlet valve.

18. The blood pump device of any one of claims 1-17, wherein the inlet valve is operable in an open state and a closed state.

19. The blood pump device of claim 18, wherein when the inlet valve is in the closed state, the leaflets define an aperture therebetween, wherein the guidewire extends through the aperture.

19. The blood pump device of claim 18, wherein a shape of the aperture is complementary to a cross-sectional profile of the guidewire or a cross-sectional profile of a guidewire housing member that receives the guidewire.

20. The blood pump device of any one of claims 1-19, further comprising a rigid support member that receives the guidewire, a guidewire housing member that receives the guidewire, or both.

21. The blood pump device of claim 20, wherein the rigid support member extends through the inlet valve.

22. The blood pump device of any one of claims 1-21, wherein the conduit comprises at least one membrane.

23. The blood pump device of claim 22, wherein the conduit further comprises an expandable support and the at least one membrane is covering at least a portion of an inner surface or outer surface of the expandable support.

24. The blood pump device of claim 23, wherein at least one of the plurality of leaflets is joined to the at least one membrane.

25. The blood pump device of claim 23 or 24, wherein the at least one membrane comprises an inner membrane covering the inner surface of the expandable support and an outer membrane covering the outer surface of the expandable support, and at least one of the plurality of leaflets is joined to the inner membrane or the outer membrane, or both.

26. The blood pump device of claim 25, wherein a proximal end of the expandable support is coupled to a catheter extending through the pump body.

27. The blood pump device of claim 25 or 26, wherein a distal end of the expandable support is longitudinally movable relative to the catheter.

28. The blood pump device of any one of claims 1-27, wherein the pump body further comprises a volume displacement member arranged in the conduit and having an expandable volume cyclically operable between an expansion phase and a contraction phase.

29. The blood pump device of claim 28, wherein a distalmost end of the expandable volume of the volume displacement member is separated from the inlet valve by a distance of between about 1 mm and about 35 mm.

30. The blood pump device of claim 28, wherein at least a portion of the volume displacement member extends through the inlet valve.

31. The blood pump device of any one of claims 28-30, wherein the volume displacement member comprises a balloon.

32. The blood pump device of any one of claims 1-31, wherein the inlet valve has an upstream side and a downstream side, and is operable in a valve cycle comprising:
an open state in which the leaflets permit fluid flow between the upstream side and the downstream side; and
a closed state in which the leaflets substantially prevent fluid flow from the downstream side to the upstream side.

33. The blood pump device of claim 32, wherein the inlet valve is configured to maintain the closed state under a pressure differential of up to at least about 500 mmHg from the downstream side to the upstream side, without allowing fluid leakage from the downstream side to the upstream side.

34. The blood pump device of claim 33, wherein the valve is configured to maintain the closed state under a pressure differential of up to at least about 700 mmHg from the downstream side to the upstream side, without allowing blood leakage from the downstream side to the upstream side.

35. The blood pump device of any one of claims 32-34, wherein the valve is configured to repeatedly cycle between the open state and the closed state at a frequency of between about 500 beats per minute and about 5000 beats per minute.

36. The blood pump device of claim 35, wherein the valve is configured to be cyclically operated at a frequency of between about 1000 beats per minute and about 5000 beats per minute.

37. The blood pump device of any one of claims 32-36, wherein the valve is configured to transition from the closed state to the open state over no more than about 20% of the duration of the valve cycle.

38. A method, comprising:
positioning a blood pump device in the circulatory system of a patient, wherein the blood pump device comprises:
a conduit having an inflow region and an outflow region:
a volume displacement member arranged in the conduit; and
an inlet valve arranged in the inflow region and comprising a plurality of leaflets, an upstream side, and a downstream side; and
cyclically operating the volume displacement member between an expansion phase and a contraction phase;
wherein the inlet valve transitions between an open state during the contraction phase to allow blood flow into the conduit, and a closed state during the expansion phase in which the valve substantially prevents blood flow out of the conduit through the inflow region, and
wherein during the expansion phase, the inlet valve maintains the closed state under a pressure differential of up to at least about 500 mmHg from the downstream side to the upstream side, without allowing blood leakage from the downstream side to the upstream side.

39. The method of claim 38, wherein the valve maintains the closed state under a pressure differential of up to at least about 700 mmHg from the downstream side to the upstream side, without allowing blood leakage from the downstream side to the upstream side.

40. The method of claim 38 or 39, wherein positioning the blood pump device comprises positioning at least a portion of the inflow region of the conduit in a left ventricle of the patient.

41. The method of claim 40, further comprising positioning at least a portion of the outflow region of the conduit in an aorta of the patient when at least a portion of the inflow region of the conduit is in the left ventricle.

42. The method of any one of claims 38-41, wherein positioning the blood pump device comprises positioning the inlet valve in a plane offset from a native valve plane.

43. The method of claim 42, wherein the native valve plane is an aortic valve plane.

44. The method of claim 38 or 39, wherein positioning the blood pump device comprises positioning the entire blood pump device in a blood vessel of the patient.

45. The method of any one of claims 38-49, wherein the inlet valve in its open state has a valve orifice area between about 15% and about 90% of a maximum cross-sectional area of the conduit, as measured at a location of the inlet valve.

46. The method of any one of claims 38-45, wherein the inlet valve comprises a cylindrical valve body and the plurality of leaflets are coupled to the valve body.

47. The method of claim 46, wherein the conduit has a first inner diameter and the valve body has a second inner diameter, the second inner diameter being between about 70% and about 98% of the first inner diameter.

48. The method of any one of claims 38-47, wherein cyclically operating the volume displacement member comprises cyclically operating the volume displacement member at a frequency of at least about 500 beats per minute.

49. The method of claim 48, wherein cyclically operating the volume displacement member comprises cyclically operating the volume displacement member at a frequency of between about 1000 beats per minute and about 5000 beats per minute.

50. The method of any one of claims 38-49, wherein positioning the blood pump device comprises collapsing the blood pump device in a lumen of a delivery catheter, and inserting the delivery catheter in a blood vessel.

51. The method of claim 50, wherein the lumen of the delivery catheter is no more than about 6 mm in diameter.

52. The method of claim 50 or 51, wherein positioning the blood pump device further comprises deploying the blood pump device from the lumen of the delivery catheter at a pumping location in a heart chamber or blood vessel.

53. The method of claim 52, wherein the inlet valve self-expands from a collapsed valve configuration in the lumen of the delivery catheter to an expanded valve configuration as the blood pump device is deployed.

54. The method of claim 53, wherein the inlet valve in the expanded valve configuration has an expanded diameter that is between about 1.2 and about 6 times the diameter of the lumen of the delivery catheter.

55. The method of any one of claims 38-54, wherein the valve is operable in a valve cycle comprising the open state and the closed state, and is configured to transition from the closed state to the open state over no more than about 20% of the duration of the valve cycle.

56. A valve arrangement for a blood pump device, the valve arrangement comprising:
a valve comprising a plurality of leaflets, an upstream side, and a downstream side, wherein the valve is operable in:
an open state in which the leaflets permit fluid flow between the upstream side and the downstream side; and
a closed state in which the leaflets substantially prevent fluid flow from the downstream side to the upstream side,
wherein the valve is configured to maintain the closed state under a pressure differential of up to at least about 500 mmHg from the downstream side to the upstream side, without allowing fluid leakage from the downstream side to the upstream side.

57. The valve arrangement of claim 56, wherein the valve is configured to maintain the closed state under a pressure differential of up to at least about 700 mmHg from the downstream side to the upstream side, without allowing fluid leakage from the downstream side to the upstream side.

58. The valve arrangement of claim 56 or 57, wherein the valve is configured to be cyclically operated between the open state and the closed state at a frequency of at least about 500 beats per minute.

59. The valve arrangement of claim 58, wherein the valve is configured to be cyclically operated at a frequency of between about 1000 beats per minute and about 5000 beats per minute.

60. The valve arrangement of any one of claims 56-59, wherein the valve is cyclically operable in a valve cycle comprising the open state and the closed state, wherein the valve is configured to transition from the closed state to the open state over no more than about 20% of the duration of the valve cycle.

61. The valve arrangement of any one of claims 56-60, wherein at least one leaflet comprises a base region comprising a first bending stiffness and an edge region comprising a second bending stiffness, wherein the second bending stiffness is lower than the first bending stiffness.

62. The valve arrangement of any one of claims 56-61, wherein the base region comprises a first leaflet thickness and the edge region comprises a second leaflet thickness, wherein the first leaflet thickness is less than the second leaflet thickness.

63. The valve arrangement of any one of claims 56-62, wherein the base region comprises a first material, and the edge region comprises a second material, wherein the second material has a greater durometer than the first material.

64. The valve arrangement of any one of claims 56-63, wherein at least one leaflet comprises multiple layers of material.

65. The valve arrangement of any one of claims 56-64, wherein the inlet valve has an outer diameter of about 25 mm or less.

66. A blood pump device comprising:
a conduit having an inflow region and an outflow region;
a volume displacement member arranged in the conduit; and
the valve arrangement of any one of claims 56-65, wherein the valve arrangement is positioned in the inflow region of the conduit.

67. The blood pump device of claim 66, wherein the conduit comprises an expandable support and at least one membrane covering at least a portion of the expandable support.

68. The blood pump device of claim 67, wherein the at least one membrane covers an inner surface or outer surface of the expandable support.

69. The blood pump device of claim 67 or 68, wherein at least one of the plurality of leaflets is joined to the at least one membrane.

70. The blood pump device of claim 68 or 69, wherein the at least one membrane comprises an inner membrane covering the inner surface of the expandable support and an outer membrane covering the outer surface of the expandable support, and at least one of the plurality of leaflets is joined to the inner membrane or the outer membrane, or both.

71. The blood pump device of any one of claims 66-70, further comprising a tubular member extending longitudinally through the conduit and between the leaflets of the valve.

72. The blood pump device of claim 71, wherein the tubular member is configured to slidably receive a guidewire.

73. The blood pump device of claim 71 or 72, wherein the leaflets are configured to coapt with an outer surface of the tubular member.

74. The blood pump device of claim 73, wherein at least one leaflet has an inner edge portion complementary with a cross-sectional profile of the tubular member.

75. The blood pump device of any one of claims 71-74, wherein the tubular member is configured to support the leaflets to substantially inhibit prolapsing of the leaflets.

76. The blood pump device of any one of claims 71-75, wherein the tubular member extends through an interior of the volume displacement member.

77. The blood pump device of any one of claims 66-76, wherein the valve in its open state has a valve orifice area between about 15% and about 90% of a maximum cross-sectional area of the conduit, as measured at a location of the inlet valve.

78. The blood pump device of any one of claims 66-77, wherein the valve comprises a cylindrical valve body and the plurality of leaflets are coupled to the valve body.

79. The blood pump device of claim 78, wherein the conduit has a first inner diameter and the valve body has a second inner diameter, the second inner diameter being between about 70% and about 98% of the first inner diameter.

80. The blood pump device of any one of claims 66-79, wherein the blood pump device is collapsible into a lumen of a delivery catheter.

81. The blood pump device of claim 80, wherein the lumen of the delivery catheter is no more than about 6 mm in diameter.

82. The blood pump device of claim 81, wherein the valve is configured to self-expand from a collapsed valve configuration to an expanded valve configuration.

83. The blood pump device of claim 82, wherein the valve in the expanded valve configuration has an expanded diameter that is between about 1.2 and about 6 times the diameter of the lumen of the delivery catheter.

84. A method, comprising:
positioning a blood pump device in the circulatory system of a patient, wherein the blood pump device comprises:
a conduit having an inflow region and an outflow region;
a volume displacement member arranged in the conduit; and
an inlet valve arranged in the inflow region and comprising a plurality of leaflets; and
cyclically operating the volume displacement member between an expansion phase and a contraction phase,
wherein the inlet valve transitions between an open state during the contraction phase to allow blood flow into the conduit, and a closed state during the expansion phase in which the valve substantially prevents blood flow out of the conduit through the inflow region, and
wherein the valve repeatedly cycles between the open state and the closed state at a frequency of between about 500 beats per minute and about 5000 beats per minute.

85. The method of claim 84, wherein the valve is cyclically operated at a frequency of between about 1000 beats per minute and about 5000 beats per minute.

86. The method of claim 84 or 85, wherein the valve remains in the open state during at least a portion of the expansion phase of the volume displacement member.

87. The method of any one of claims 84-86, wherein during the expansion phase, the inlet valve maintains the closed state under a pressure differential of up to at least about 500 mmHg from the downstream side to the upstream side, without allowing blood leakage from the downstream side to the upstream side.

88. The method of claim 87, wherein the valve maintains the closed state under a pressure differential of up to at least about 700 mmHg from the downstream side to the upstream side, without allowing blood leakage from the downstream side to the upstream side.

89. The method of any one of claims 84-88, wherein positioning the blood pump device comprises positioning at least a portion of the inflow region of the conduit in a left ventricle of the patient.

90. The method of claim 89, further comprising positioning at least a portion of the outflow region of the conduit in an aorta of the patient when at least a portion of the inflow region of the conduit is in the left ventricle.

91. The method of any one of claims 84-90, wherein positioning the blood pump device comprises positioning the inlet valve in a plane offset from a native valve plane.

92. The method of claim 91, wherein the native valve plane is an aortic valve plane.

93. The method of any one of claims 84-88, wherein positioning the blood pump device comprises positioning the entire blood pump device in a blood vessel of the patient.

94. The method of any one of claims 84-93, wherein the inlet valve in its open state has a valve orifice area between about 15% and about 90% of a maximum cross-sectional area of the conduit, as measured at a location of the inlet valve.

95. The method of any one of claims 84-94, wherein the inlet valve comprises a cylindrical valve body and the plurality of leaflets are coupled to the valve body.

96. The method of claim 95, wherein the conduit has a first inner diameter and the valve body has a second inner diameter, the second inner diameter being at least between about 70% and about 98% of the first inner diameter.

97. The method of any one of claims 84-96, wherein cyclically operating the volume displacement member comprises cyclically operating the volume displacement member at a frequency of at least about 500 beats per minute.

98. The method of claim 97, wherein cyclically operating the volume displacement member comprises cyclically operating the volume displacement member at a frequency of between about 1000 beats per minute and about 5000 beats per minute.

99. The method of any one of claims 84-98, wherein positioning the blood pump device comprises collapsing the blood pump device in a lumen of a delivery catheter, and inserting the delivery catheter in a blood vessel.

100. The method of claim 99, wherein the lumen of the delivery catheter is no more than about 6 mm in diameter.

101. The method of claim 99 or 100, wherein positioning the blood pump device further comprises deploying the blood pump device from the lumen of the delivery catheter at a pumping location in a heart chamber or blood vessel.

102. The method of claim 101, wherein the inlet valve self-expands from a collapsed valve configuration in the lumen of the delivery catheter to an expanded valve configuration as the blood pump device is deployed.

103. The method of claim 102, wherein the inlet valve in the expanded valve configuration has an expanded diameter that is between about 1.2 and about 6 times the diameter of the lumen of the delivery catheter.

104. The method of any one of claims 84-103, wherein the valve operates over a valve cycle comprising the open state and the closed state, and the valve transitions from the closed state to the open state over no more than about 20% of the duration of the valve cycle.

105. A valve arrangement for a blood pump device, the valve arrangement comprising:
a valve comprising a plurality of leaflets, an upstream side, and a downstream side, wherein the valve is operable in:
an open state in which the leaflets permit fluid flow between the upstream side and the downstream side; and
a closed state in which the leaflets substantially prevent fluid flow from the downstream side to the upstream side,
wherein the valve is configured to repeatedly cycle between the open state and the closed state at a frequency of between about 500 beats per minute and about 5000 beats per minute.

106. The valve arrangement of claim 105, wherein the valve is configured to be cyclically operated at a frequency of between about 1000 beats per minute and about 5000 beats per minute.

107. The valve arrangement of claim 105 or 106, wherein the valve is configured to maintain the closed state under a pressure differential of up to at least about 500 mmHg from the downstream side to the upstream side, without allowing fluid leakage from the downstream side to the upstream side.

108. The valve arrangement of claim 107, wherein the valve is configured to maintain the closed state under a pressure differential of up to at least about 700 mmHg from the downstream side to the upstream side, without allowing fluid leakage from the downstream side to the upstream side.

109. The valve arrangement of any one of claims 105-108, wherein the valve is cyclically operable in a valve cycle comprising the open state and the closed state, wherein the valve is configured to transition from the closed state to the open state over no more than about 20% of the duration of the valve cycle.

110. The valve arrangement of any one of claims 105-109, wherein at least one leaflet comprises a base region comprising a first bending stiffness and an edge region comprising a second bending stiffness, wherein the second bending stiffness is lower than the first bending stiffness.

111. The valve arrangement of any one of claims 105-110, wherein the base region comprises a first leaflet thickness and the edge region comprises a second leaflet thickness, wherein the first leaflet thickness is less than the second leaflet thickness.

112. The valve arrangement of any one of claims 105-111, wherein the base region comprises a first material, and the edge region comprises a second material, wherein the second material has a greater durometer than the first material.

113. The valve arrangement of any one of claims 105-112, wherein at least one leaflet comprises multiple layers of material.

114. The valve arrangement of any one of claims 105-113, wherein the inlet valve has an outer diameter of about 25 mm or less.

115. A blood pump device comprising:
a conduit having an inflow region and an outflow region;
a volume displacement member arranged in the conduit; and
the valve arrangement of any one of claims 105-114, wherein the valve arrangement is positioned in the inflow region of the conduit.

116. The blood pump device of claim 115, wherein the conduit comprises an expandable support and at least one membrane covering at least a portion of the expandable support.

117. The blood pump device of claim 116, wherein the at least one membrane covers an inner surface or outer surface of the expandable support.

118. The blood pump device of claim 116 or 117, wherein at least one of the plurality of leaflets is joined to the at least one membrane.

119. The blood pump device of claim 117 or 118, wherein the at least one membrane comprises an inner membrane covering the inner surface of the expandable support and an outer membrane covering the outer surface of the expandable support, and at least one of the plurality of leaflets is joined to the inner membrane or the outer membrane, or both.

120. The blood pump device of any one of claims 115-119, further comprising a tubular member extending longitudinally through the conduit and between the leaflets of the valve.

121. The blood pump device of claim 120, wherein the tubular member is configured to slidably receive a guidewire.

122. The blood pump device of claim 120 or 121, wherein the leaflets are configured to coapt with an outer surface of the tubular member.

123. The blood pump device of claim 122, wherein at least one leaflet has an inner edge portion complementary with a cross-sectional profile of the tubular member.

124. The blood pump device of any one of claims 120-123, wherein the tubular member is configured to support the leaflets to substantially inhibit prolapsing of the leaflets.

125. The blood pump device of any one of claims 120-124, wherein the tubular member extends through an interior of the volume displacement member.

126. The blood pump device of any one of claims 115-125, wherein the valve in its open state has a valve orifice area between about 15% and about 90% of a maximum cross-sectional area of the conduit, as measured at a location of the inlet valve.

127. The blood pump device of any one of claims 115-126, wherein the valve comprises a cylindrical valve body and the plurality of leaflets are coupled to the valve body.

128. The blood pump device of claim 127, wherein the conduit has a first inner diameter and the valve body has a second inner diameter, the second inner diameter being at least between about 70% and about 98% of the first inner diameter.

129. The blood pump device of any one of claims 115-128, wherein the blood pump device is collapsible into a lumen of a delivery catheter.

130. The blood pump device of claim 129, wherein the lumen of the delivery catheter is no more than about 6 mm in diameter.

131. The blood pump device of claim 130, wherein the valve is configured to self-expand from a collapsed valve configuration to an expanded valve configuration.

132. The blood pump device of claim 131, wherein the valve in the expanded valve configuration has an expanded diameter that is between about 1.2 and about 6 times the diameter of the lumen of the delivery catheter.

133. A method, comprising:
positioning a blood pump device in the circulatory system of a patient, wherein the blood pump device comprises:
a conduit having an inflow region and an outflow region;
a volume displacement member arranged in the conduit; and
an inlet valve arranged in the inflow region and comprising a plurality of leaflets; and
cyclically operating the volume displacement member between an expansion phase and a contraction phase,
wherein the inlet valve transitions between an open state during the contraction phase to allow blood flow into the conduit, and a closed state during the expansion phase in which the valve substantially prevents blood flow out of the conduit through the inflow region, and
wherein the inlet valve transitions from the closed state to the open state over no more than about 20% of the duration of the valve cycle.

134. The method of claim 133, wherein the inlet valve remains in the open state over at least about 50% of the duration of the valve cycle.

135. The method of claim 133 or 134, wherein during the expansion phase, the inlet valve maintains the closed state under a pressure differential of up to at least about 500 mmHg from the downstream side to the upstream side, without allowing blood leakage from the downstream side to the upstream side.

136. The method of claim 135, wherein the valve maintains the closed state under a pressure differential of up to at least about 700 mmHg from the downstream side to the upstream side, without allowing blood leakage from the downstream side to the upstream side.

137. The method of any one of claims 133-136, wherein positioning the blood pump device comprises positioning at least a portion of the inflow region of the conduit in a left ventricle of the patient.

138. The method of claim 137, further comprising positioning at least a portion of the outflow region of the conduit in an aorta of the patient when at least a portion of the inflow region of the conduit is in the left ventricle.

139. The method of any one of claims 133-138, wherein positioning the blood pump device comprises positioning the inlet valve in a plane offset from a native valve plane.

140. The method of claim 139, wherein the native valve plane is an aortic valve plane.

141. The method of any one of claims 133-136, wherein positioning the blood pump device comprises positioning the entire blood pump device in a blood vessel of the patient.

142. The method of any one of claims 133-141, wherein the inlet valve in its open state has a valve orifice area between about 15% and about 90% of a maximum cross-sectional area of the conduit, as measured at a location of the inlet valve.

143. The method of any one of claims 133-142, wherein the inlet valve comprises a cylindrical valve body and the plurality of leaflets are coupled to the valve body.

144. The method of claim 143, wherein the conduit has a first inner diameter and the valve body has a second inner diameter, the second inner diameter being at least between about 70% and about 98% of the first inner diameter.

145. The method of any one of claims 133-144, wherein cyclically operating the volume displacement member comprises cyclically operating the volume displacement member at a frequency of at least about 500 beats per minute.

146. The method of claim 145, wherein cyclically operating the volume displacement member comprises cyclically operating the volume displacement member at a frequency of between about 1000 beats per minute and about 5000 beats per minute.

147. The method of any one of claims 133-146, wherein positioning the blood pump device comprises collapsing the blood pump device in a lumen of a delivery catheter, and inserting the delivery catheter in a blood vessel.

148. The method of claim 147, wherein the lumen of the delivery catheter is no more than about 6 mm in diameter.

149. The method of claim 147 or 148, wherein positioning the blood pump device further comprises deploying the blood pump device from the lumen of the delivery catheter at a pumping location in a heart chamber or blood vessel.

150. The method of claim 149, wherein the inlet valve is configured to self-expand from a collapsed valve configuration in the lumen of the delivery catheter to an expanded valve configuration as the blood pump device is deployed.

151. The method of claim 150, wherein the inlet valve in the expanded valve configuration has an expanded diameter that is between about 1.2 and about 6 times the diameter of the lumen of the delivery catheter.

152. The method of any one of claims 133-151, wherein the valve repeatedly cycles between the open state and the closed state at a frequency of between about 500 beats per minute and about 5000 beats per minute.

153. The method of claim 152, wherein the valve is configured to be cyclically operated at a frequency of between about 1000 beats per minute and about 5000 beats per minute.

154. The method of any one of claims 133-153, wherein during the expansion phase, the inlet valve maintains the closed state under a pressure differential of up to at least about 500 mmHg from the downstream side to the upstream side, without allowing blood leakage from the downstream side to the upstream side.

155. The method of claim 154, wherein the valve maintains the closed state under a pressure differential of up to at least about 700 mmHg from the downstream side to the upstream side, without allowing blood leakage from the downstream side to the upstream side

156. A valve arrangement for a blood pump device, the valve arrangement comprising:
a valve comprising a plurality of leaflets, an upstream side, and a downstream side, wherein the valve is operable in a valve cycle including:
an open state in which the leaflets permit fluid flow between the upstream side and the downstream side; and
a closed state in which the leaflets substantially prevent fluid flow from the downstream side to the upstream side,
wherein the valve is configured to transition from the closed state to the open state over no more than about 20% of the duration of the valve cycle.

157. The valve arrangement of claim 156, wherein the valve is configured to maintain the closed state under a pressure differential of up to at least about 500 mmHg from the downstream side to the upstream side, without allowing fluid leakage from the downstream side to the upstream side.

158. The valve arrangement of claim 156 or 157, wherein the valve is configured to maintain the closed state under a pressure differential of up to at least about 700 mmHg from the downstream side to the upstream side, without allowing fluid leakage from the downstream side to the upstream side.

159. The valve arrangement of any one of claims 156-158, wherein the valve is configured to be cyclically operated between the open state and the closed state at a frequency of at least about 500 beats per minute.

160. The valve arrangement of claim 159, wherein the valve is configured to be cyclically operated at a frequency of between about 1000 beats per minute and about 5000 beats per minute.

161. The valve arrangement of any one of claims 156-160, wherein at least one leaflet comprises a base region comprising a first bending stiffness and an edge region comprising a second bending stiffness, wherein the second bending stiffness is lower than the first bending stiffness.

162. The valve arrangement of any one of claims 156-161, wherein the base region comprises a first leaflet thickness and the edge region comprises a second leaflet thickness, wherein the first leaflet thickness is less than the second leaflet thickness.

163. The valve arrangement of any one of claims 156-162, wherein the base region comprises a first material, and the edge region comprises a second material, wherein the second material has a greater durometer than the first material.

164. The valve arrangement of any one of claims 156-163, wherein at least one leaflet comprises multiple layers of material.

165. The valve arrangement of any one of claims 156-164, wherein the inlet valve has an outer diameter of about 25 mm or less.

166. A blood pump device comprising:
a conduit having an inflow region and an outflow region;
a volume displacement member arranged in the conduit; and
the valve arrangement of any one of claims 156-165, wherein the valve arrangement is positioned in the inflow region of the conduit.

167. The blood pump device of claim 166, wherein the conduit comprises an expandable support and at least one membrane covering at least a portion of the expandable support.

168. The blood pump device of claim 167, wherein the at least one membrane covers an inner surface or outer surface of the expandable support.

169. The blood pump device of claim 167 or 168, wherein at least one of the plurality of leaflets is joined to the at least one membrane.

170. The blood pump device of claim 168 or 169, wherein the at least one membrane comprises an inner membrane covering the inner surface of the expandable support and an outer membrane covering the outer surface of the expandable support, and at least one of the plurality of leaflets is joined to the inner membrane or the outer membrane, or both.

171. The blood pump device of any one of claims 166-170, further comprising a tubular member extending longitudinally through the conduit and between the leaflets of the valve.

172. The blood pump device of claim 171, wherein the tubular member is configured to slidably receive a guidewire.

173. The blood pump device of claim 171 or 172, wherein the leaflets are configured to coapt with an outer surface of the tubular member.

174. The blood pump device of claim 173, wherein at least one leaflet has an inner edge portion complementary with a cross-sectional profile of the tubular member.

175. The blood pump device of any one of claims 171-174, wherein the tubular member is configured to support the leaflets to substantially inhibit prolapsing of the leaflets.

176. The blood pump device of any one of claims 171-175, wherein the tubular member extends through an interior of the volume displacement member.

177. The blood pump device of any one of claims 166-176, wherein the valve in its open state has a valve orifice area between about 15% and about 90% of a maximum cross-sectional area of the conduit, as measured at a location of the inlet valve.

178. The blood pump device of any one of claims 166-177, wherein the valve comprises a cylindrical valve body and the plurality of leaflets are coupled to the valve body.

179. The blood pump device of claim 178, wherein the conduit has a first inner diameter and the valve body has a second inner diameter, the second inner diameter being at least between about 70% and about 98% of the first inner diameter.

180. The blood pump device of any one of claims 166-179, wherein the blood pump device is collapsible into a lumen of a delivery catheter.

181. The blood pump device of claim 180, wherein the lumen of the delivery catheter is no more than about 6 mm in diameter.

182. The blood pump device of claim 181, wherein the valve is configured to self-expand from a collapsed valve configuration to an expanded valve configuration.

183. The blood pump device of claim 182, wherein the valve in the expanded valve configuration has an expanded diameter that is between about 1.2 and about 6 times the diameter of the lumen of the delivery catheter.

184. A blood pump device comprising:
a pump body comprising:
a conduit having an inflow region and an outflow region, wherein the conduit comprises an expandable support and a membrane covering at least a portion of the expandable support;
a balloon arranged in the conduit and cyclically operable between an expansion phase and a contraction phase; and
an inlet valve arranged in the inflow region and comprising a plurality of leaflets,
wherein the inlet valve transitions between an open state during the contraction phase to allow blood flow into the conduit, and a closed state during the expansion phase in which the valve substantially prevents blood flow out of the conduit through the inflow region,
wherein each leaflet comprises a base region comprising a first bending stiffness and an edge region comprising a second bending stiffness lower than the first bending stiffness, and
wherein each leaflet is joined to the membrane of the conduit;
a guidewire extending through the inlet valve; and
a tubular member configured to slidably receive the guidewire, wherein the tubular member extends through the inlet valve and is configured to support the leaflets to substantially inhibit prolapsing of the leaflets.

185. The blood pump device of claim 184, wherein the inlet valve is configured to maintain the closed state under a pressure differential of up to at least about 500 mmHg from a downstream side of the valve to an upstream side of the valve, without allowing fluid leakage from the downstream side to the upstream side.

186. The blood pump device of claim 185, wherein the valve is configured to maintain the closed state under a pressure differential of up to at least about 700 mmHg from the downstream side to the upstream side, without allowing fluid leakage from the downstream side to the upstream side.

187. The blood pump device of any one of claims 184-186, wherein the valve is configured to be cyclically operated between the open state and the closed state at a frequency of at least about 500 beats per minute.

188. The blood pump device of any one of claims 184-187, wherein the valve is configured to be cyclically operated at a frequency of between about 1000 beats per minute and about 5000 beats per minute.

189. The blood pump device of any one of claims 184-188, wherein the valve is cyclically operable in a valve cycle comprising the open state and the closed state, wherein the valve is configured to transition from the closed state to the open state over no more than about 20% of the duration of the valve cycle.

190. The blood pump device of any one of claims 184-189, wherein the valve in its open state has a valve orifice area between about 15% and about 90% of a maximum cross-sectional area of the conduit, as measured at a location of the inlet valve.

191. The blood pump device of any one of claims 184-190, wherein the blood pump device is collapsible into a lumen of a delivery catheter.

192. The blood pump device of claim 191, wherein the lumen of the delivery catheter is no more than about 6 mm in diameter.

193. The blood pump device of claim 192, wherein the valve is configured to self-expand from a collapsed valve configuration to an expanded valve configuration.

194. The blood pump device of claim 193, wherein the valve in the expanded valve configuration has an expanded diameter that is at least between about 1.2 and about 6 times the diameter of the lumen of the delivery catheter.
